(19) United States
(12) Patent Application Publication
(10) Pub. No.: US 2011/0076318 A1
(43) Pub. Date: Mar. 31, 2011

(54) RETINOID-CONTAINING SUSTAINED RELEASE INTRAOCULAR IMPLANTS AND RELATED MATTERS

(75) Inventors: Patrick M. Hughes, Aliso Viejo, CA (US); Orest Olejnik, Coto de Caza, CA (US); Glenn Tony Huang, Fremont, CA (US); Joan-En Chang-Lin, Tustin, CA (US); Thierry Nivaggioli, Los Altos Hills, CA (US); JaneGuo Shiah, Irvine, CA (US); Michele Boix, Cantaron (FR); Christian Sarrazin, Pegomas (FR)

(73) Assignee: Allergan, Inc.

(21) Appl. No.: 12/962,427

(22) Filed: Dec. 7, 2010

Related U.S. Application Data
(63) Continuation of application No. 12/388,400, filed on Feb. 18, 2009, now abandoned.
Application No. 12/388,400 is a division of application No. 11/119,024, filed on Apr. 29, 2005, now abandoned.
(60) Provisional application No. 60/567,339, filed on Apr. 30, 2004.
(60) Provisional application No. 60/629,928, filed on Nov. 22, 2004.

(51) Int. Cl. A61M 31/00; A61K 31/4436; A61P 27/02

(52) U.S. Cl. 424/425; 514/337

(57) ABSTRACT

Biocompatible intraocular implants include a retinoid component and a biodegradable polymer that is effective to facilitate release of the retinoid component into an eye for an extended period of time. The therapeutic agents of the implants may be associated with a biodegradable polymer matrix, such as a matrix that is substantially free of a polyvinyl alcohol. The implants may be placed in an eye to treat or reduce the occurrence of one or more ocular conditions, such as retinal damage, including glaucoma and proliferative vitreoretinopathy.

### RELATED APPLICATIONS

This is a continuation of divisional of application Ser. No. 12/388,400 filed Feb. 18, 2009 which is a divisional of 11/119,024 filed Apr. 29, 2005, which claims the benefit of U.S. Provisional Application No. 60/567,339, filed Apr. 30, 2004, and U.S. Provisional Application No. 60/629,928, filed Nov. 22, 2004, the disclosures of all of which are hereby incorporated by reference in their entireties.

### BACKGROUND

The present invention generally relates to devices and methods to treat an eye of a patient, and more specifically to intraocular implants that provide extended release of a therapeutic agent to an eye in which the implant is placed, and to methods of making and using such implants, for example, to treat or reduce one or more symptoms of an ocular condition.

Retinoid drugs exert their therapeutic activity by stimulating, blocking or inhibiting the biological activities of either or both of the retinoid-associated nuclear receptors RAR (retinoic acid receptors) and RXR (retinoid X receptors). Although not wishing to be limited by any particular theory, each of these receptors is thought to undergo a conformational change when a cognitive agonist binds the receptor. This conformational change then results in the receptor stimulating or inhibiting the expression of a set of particular genes. This process is termed transactivation. In addition, there are myriad ligand-mediated effects, such as involvement in the stimulation or mediation of cellular phosphrylation cascades, which may not be transactivational events.

Retinoid drugs formulated for oral delivery, for example, RAR agonists which affect one or more retinoic acid receptors or RARs, are currently used for the treatment of psoriasis (acitretin and etretinate) and acne (isotretinoin). These RAR agonists are known to be associated with a large diversity of side effects at the doses necessary for acceptable or substantially optimal or optimal therapeutic activity, including, without limitation, side effects similar to those commonly associated with hypervitaminosis A, metabolic and nutritional side effects, whole body side effects, endocrine side effects, hemic and lymphatic system side effects, digestive system side effects, ocular side effects, cardiovascular side effects, nervous system side effects, psychiatric side effects, typical retinoid toxicity side effects, respiratory system side effects, ear side effects, gastrointestinal tract side effects, and urinary system side effects. The side effects associated with the use of these drugs are of considerable clinical significance and often preclude the use of these drugs in many patients or necessitate the close monitoring of liver enzymes, blood chemistries, and the like.

In addition to the RAR agonists, RXR agonists, such as bexarotene, are also associated with many of the classic retinoid side effects, such as elevations of liver enzymes and blood lipids. Hypothyroidism also seems to be a relatively common feature of RXR-active retinoids and this condition is itself associated with many significant and serious complaints including mental confusion and depression.

It should be noted that RAR and RXR are each known to form dimers either between themselves (in the case of RXR-RXR homodimers) or with other receptors. Thus RXR may form dimers with receptors such as thyroid receptor (TR), vitamin D receptor and PPAR (peroxisome proliferator-activated receptor) in addition to forming a homodimer. Thus, retinoid receptor dimers may include RXR-RXR homodimers, or heterodimers such as RXR-RAR, TXR-TR or RXR-PPAR. RAR does not appear to form homodimers and is apparently invariably paired with RXR.

The RAR and RXR receptors each has three major subtypes; thus, RAR receptors comprise RAR alpha, RAR beta, and RAR gamma. Similarly, RXR receptors comprise RXR alpha, RXR beta, and RXR gamma.

Tretinoin is an endogenous retinoid, which is metabolized readily to isotretinoin and other metabolites, including 9-cis retinoic acid. Tretinoin binds and transactivates both RAR and RXR, as does isotretinoin and 9-cis-retinoic acid. Tretinoin (Vesanoid) is used systemically for the treatment of acute promyelocytic anemia. The side effects of systemic tretinoin are typical of those accompanying systemic retinoid use generally, and appear to represent both RAR and RXR-type side effects.

Many retinoid drugs are formulated for oral delivery, for example, RAR agonists such as isotretinoin (Accutane), RXR agonists such as bexarotene (Targretin) and RAR, RXR dual agonists such as acitretin (Soriatane). For these retinoids, peak blood concentrations vary depending upon when the oral drug was administered relative to meals; however the time to peak blood concentration does not appear to be affected. In the case of isotretinoin the total dose of the drug must be more than doubled to reach the same peak blood concentration following a high fat meal as compared to the fasted state. This is seen as a significant disadvantage for these potent oral retinoids since the drug-absorption profile can drastically change depending upon the fasted or fed state of the patient.

Non-compliance with prescribed treatment regimens and oral administration directions could undermine the effectiveness of these retinoids when treating disease states, such as, without limitation, for retinal ocular conditions e.g. age related macular degeneration, diabetic neuropathy and the like. Moreover, retinoid absorption variability can lead not only to reduced therapeutic efficacy resulting from fluctuations of therapeutic drug-blood levels, but can also cause unwarranted drug side effects due in inadvertently high tissue exposure. It is therefore important, and indeed reinforced by prescribing physicians and the US Food and Drug Administration, that oral doses of retinoids be taken with food.

Proliferative vitreoretinopathy (PVR) remains the major cause of failure in retinal reattachment surgery. The pathophysiology of PVR involves the migration, dedifferentiation, and proliferation of retinal pigmented epithelial (RPE) cells and glial cells into the vitreous followed by epiretinal membrane formation. Contraction of the cellular membrane leads to the breakdown of the blood-retinal barrier and traction retinal detachment.

The onset of PVR is heralded by the migration of RPE cells into the vitreous. RPE dedifferentiation and proliferation occurs in PVR, proliferative diabetic retinopathy (PDR) and choroidal neovascularization. Several growth factors and cytokines have been implicated in the proliferative process and include: aFGF, bFGF, epidermal growth factor, IGF-I, TGF-beta, interleukin 1, 6 and 8 (IL-1, IL-6,IL-6), interferon gamma (IFN gamma), epidermal growth factor, macrophage colony stimulating factor(M-CSF) and monocyte chemotactic factor-1 (MCP-1). Pharmacological treatment of PVR is generally aimed at downstream sequelae of RPE proliferation, specifically membrane formation and inflammatory infiltration. This includes the use of corticosteroids to prevent the inflammatory component and macrophage recruitment and cytostatics to prevent the proliferative phase. Triamcinolone acetonide and dexamethasone have both been studied to prevent traction retinal detachment from PVR. The corticosteroids are to some degree effective but carry significant side effects including cataract formation and elevation of IOP. Numerous cytostatic agents have also been examined and include: cytarabine, 5-fluorouracil, daunorubicin, aclacinomycin A, BCNU, N,N dimethyladriamycin, and taxol. These agents have been shown to inhibit traction retinal detachment in animal models of PVR but carry significant side effects ranging from retinal disruption to carcinogenicity.

The use of retinoic acid (RA) and other retinoids has been investigated and show promise in fulfilling the PVR treatment needs. Retinoids treat the underlying pathology of PVR, RPE dedifferentiation and proliferation, as well as the downstream effects. Retinoids have an antiproliferative effect on epithelial, mesenchymal and neoplastic cells. All trans-retinoic acid (RA) is known to inhibit retinal pigmented epithelial proliferation. It has also been suggested that retinoids might be able to enhance density-dependent growth regulation in RPE. Studies have shown that RA prevents RPE proliferation in-vitro in a biphasic manner with IC₅₀ of 10 pM and 17 nM. Retinoic acid also inhibits human RPE expression of stromelysin. It is believed that this prevents the cleaving of proteins in the extracellular space that leads to RPE dispersion. Additionally, RA has been shown to modulate the effect of bFGF in cultured RPE cells. RA inhibited bFGF stimulated RPE proliferation. Scatchard plot analysis suggested that RA decreased the number of bFGF binding sites on the RPE cells. Recycling of retinoids is required for maintenance of normal visual function as they play an important role in visual transduction. The lack of retinoid input could contribute to RPE dedifferentiation, migration, and proliferation processes that occur after retinal detachment and where PVR has been previously been involved results in poor return of vision after retinal reattachment.

Animal models of PVR have demonstrated the relative safety and efficacy of retinoids in the prevention of traction retinal detachment. Ten and 15 μg of RA in 1-% hyaluronic acid and BSS reduced traction retinal detachment in an animal model of PVR. Five to ten μg of RA and 13-cis-retinoic acid in a silicone oil tamponade have both been shown to effectively prevent traction retinal detachment. Histopathology and ophthalmoscopic examination indicated no ocular toxicity associated with this dose of RA. Additional studies have shown that RA concentrations up to 15 μg/mL are well tolerated with no ERG changes. The beneficial effects of retinoids in the prevention of PVR have also been shown in humans. Orally administered 13-cis-retinoic acid was effective at decreasing PVR and increasing the rate of retinal attachment in a retrospective study.

Age Related Macular Degeneration is the leading cause of blindness for individuals greater than fifty years old. The disease is heralded by the formation of focal yellow-gray lesions in Bruch's membrane. The RPE phenotype changes result in a dysregulation of the extracellular matrix synthesis and degradation. The lesions, drusen, are comprised of lipid-rich extracellular matrix components and may coalesce overtime resulting in a shallow elevation of the RPE cells. The RPE cells begin to clump, aggregate and atrophy. Degeneration of the RPE cells leads to a secondary degeneration of the overlying photoreceptors.

Retinoids may alter the phenotype of the RPE cells. Restoring RPE cell function, ECM metabolism and the intimate relationship between the RPE and photoreceptors. In addition to the RPE effects, tazarotene appears to be retinal protective in a light degeneration animal model.

Retinoids have also been shown to be effective in Stargardt's disease and to improve neural survival in rhodopsin mutant transgenic mice as well as light.

Degeneration of retinal neurons is a major cause of blindness. In glaucoma, ganglion cell death is the direct cause of blindness. In retinal degenerative disorders, including retinitis pigmentosa and age-related macular degeneration, photoreceptor death results in loss of vision. Although currently there is no effective treatment to prevent degeneration in retinal neurons, recent demonstration that retinal neurons are protected by various neurotrophic factors and small molecules indicates that pharmacological therapies for these conditions are a possibility.

In treating ocular conditions, it is important to keep in mind that the internal structures of the eye are sequestered from the general circulation of a patient by a series of highly selective barriers. These barriers prevent the rapid equilibration of compounds in the plasma with the tissues of the eye. The anatomy and physiology of the eye gives rise to the concept of the blood-aqueous and the blood-retinal barriers. Collectively these are known as the blood ocular barriers.

Access to the aqueous humor of the anterior and posterior chambers is restricted by the blood-aqueous barrier. The aqueous humor is not a simple ultrafiltrate of the blood and has a composition resulting from the combined actions of the secretory activity in the ciliary processes and the selectivity of the blood aqueous barrier. The nonpigmented cells of the ciliary body line the posterior chamber and represents part of the blood-aqueous barrier. The tight junctions joining the membranes of the cells, however, are not completely belted and this discontinuity results in intercellular pores through which solutes of intermediate size may diffuse.

The endothelial cells of the iris vessels comprise the remainder of the blood aqueous barrier. However, the cells lining the anterior surface of the iris stroma have numerous openings and present very little barrier in accessing the anterior chamber. Compounds administered systemically will penetrate the leaky vessels of the ciliary body and diffuse through the iris into the anterior chamber aqueous humor. Movement into the posterior chamber from the anterior chamber is restricted by the diaphragm like action of the iris on the lens. Many lipophilic substances such as chloramphenicol and tetracycline may penetrate readily across the blood-aqueous barrier into the posterior chamber.

The systemic administration of compounds that gain access to the vitreous via the posterior chamber is extremely inefficient. Drug must diffuse from the posterior chamber into the deeper segments of the vitreous body while competing with the parallel elimination of the posterior aqueous humor through the anterior chamber and the normal aqueous humor egress pathways. Compounds diffusing into the vitreous from the posterior chamber will develop a concentration gradient across the vitreous. This concentration gradient, however, is shallow and rapidly reversed as the aqueous humor concentration falls.

Penetration of drugs directly into the posterior segment of the eye is restricted by the blood-retinal barriers. The blood-retinal barrier is anatomically separated into an inner and outer blood barriers. The choroid lies immediately inside the sclera and is the most vascularised tissue of the posterior globe. Numerous fenestrae are present in the endothelium of the choriocapillaries resulting in very little resistance to the transport of systemic solutes into the choroid. Systemically administered compounds penetrate into the choroid in a matter of minutes and the choroid rapidly equilibrates with the plasma. Further movement of systemically borne solutes into the internal ocular structures from the choroid is restricted by the retinal pigmented epithelium (RPE). The cells of this structure are joined by zonulae occludentae intercellular junctions. The RPE is a “tight” ion transporting barrier a paracellular transport of solutes across the RPE is restricted.

The endothelium of the retinal vessels represents the inner blood-retinal barrier. The endothelial cells of the retinal vessels are completely banded by zonulae occludentae junctions preventing the paracellular transport of most blood solutes. The retinal vessels are similar to the vessels in the brain that comprise the blood-brain barrier in that they are very impermeable. The permeability of most compounds across the blood-retinal barriers is very low. Extremely lipophilic compounds, however, such as chloramphenicol and benzyl penicillin can penetrate the blood-retinal barrier achieving appreciable concentrations in the vitreous humor after systemic administration.

The lipophilicity of the compound correlates with its rate of penetration and is consistent with passive cellular diffusion. The blood retinal barrier, however, is impermeable to polar or charged compounds in the absence of a transport mechanism.

Thus, delivery of drugs to the retina, vitreous and uveal tract is typically achieved by high systemic dosing or direct intra-ocular injections.

Tazarotene, ethyl 6-[4,4-dimethylthiochroman-6-yl)ethyl]nicotinate, is a proprietary acetylenic retinoid compound developed by Allergan for psoriasis and acne vulgaris. Tazarotene is an ethyl ester prodrug that is metabolized to its active form, tazarotenic acid, by rapid deesterification in animals and human. Tazarotenic acid mediates its responses primarily through activation of nuclear retinoid receptors and has been proven to be pharmacologically active in tests using proliferative vitreoretinopathy (PVR) animal models.

The use of the retinoid. Tazarotene, is discussed in the following references: Drugs Future 28(2):208-09 (2003); Marks, “Topical tazarotene: review and re-evaluation”, Retinoids, 17(3):72-74 (2001); Phillips et al., “Efficacy of 0.1% tazarotene cream for the treatment of photodamage”, Arch Dermatol, 138(11):1486-1493 (2002); Guenther, “Optimizing treatment with topical tazarotene”, Am J Clin Dermatol, 4(3):197-202 (2003).

U.S. Pat. No. 6,713,081 discloses ocular implant devices made from polyvinyl alcohol and used for the delivery of a therapeutic agent to an eye in a controlled and sustained manner. The implants may be placed subconjunctivally or intravitreally in an eye.

Biocompatible implants for placement in the eye have also been disclosed in a number of patents, such as U.S. Pat. Nos. 4,521,210; 4,853,224; 4,997,652; 5,164,188; 5,443,505; 5,501,856; 5,766,242; 5,824,072; 5,869,079; 6,074,661; 6,331,313; 6,369,116; and 6,699,493.

It would be advantageous to provide eye implantable drug delivery systems, such as intraocular implants, and methods of using such systems, that are capable of releasing a therapeutic agent at a sustained or controlled rate for extended periods of time and in amounts with few or no negative side effects.

### SUMMARY

The present invention provides new drug delivery systems, and methods of making and using such systems, for extended or sustained drug release into an eye, for example, to achieve one or more desired therapeutic effects. The drug delivery systems are in the form of implants or implant elements that may be placed in an eye. The present systems and methods advantageously provide for extended release times of one or more therapeutic agents. Thus, the patient in whose eye the implant has been placed receives a therapeutic amount of an agent for a long or extended time period without requiring additional administrations of the agent. For example, the patient has a substantially consistent level of therapeutically active agent available for consistent treatment of the eye over a relatively long period of time, for example, on the order of at least about one week, such as between about one and about six months after receiving an implant. Such extended release times facilitate obtaining successful treatment results. The sustained local delivery of the therapeutic agent from the implant reduces the high transient concentrations associated with pulsed dosing. Furthermore, direct intravitreal administration of the implant obviates the constraints posed by the blood-retinal barrier and significantly reduces the risk of systemic toxicity.

Intraocular implants in accordance with the disclosure herein comprise a therapeutic component and a drug release sustaining component associated with the therapeutic component. In accordance with the present invention, the therapeutic component comprises, consists essentially of, or consists of, a retinoid component. For example, the therapeutic component may comprise, consist essentially of, or consist of, one or more RAR or RXR agonists, such as tazarotenic acid, or a prodrug of an RAR or RXR agonists, such as tazarotene, and the like.

A prodrug is an inactive derivative of a known active drug with enhanced delivery characteristics and therapeutic value. It is converted back to the parent compound by virtue of its enzymatic and/or chemical lability within the biologic system. The present implants may include therapeutic agents whose target tissue is in the posterior of the eye. The functional groups of the parent compound amenable to prodrug derivatization can include carboxylic acids, hydroxyl groups, amine groups or any other functionality known to be amenable to prodrug derivatization. Prodrugs include esters of hydroxyl containing groups. Other prodrugs of hydroxyl containing compounds include phosphate esters, hemiesters of dicarboxylic acids, acyloxyalkyl and ethers. Prodrugs of the amine functionality include N-Mannich Bases and Amides.

The drug release sustaining component is associated with the therapeutic component to sustain release of an amount of the retinoid component into an eye in which the implant is placed. The amount of the retinoid component is released into the eye for a period of time greater than about one week after the implant is placed in the eye and is effective in reducing or treating an ocular condition, such as proliferative vitreal retinopathy, age related macular degeneration, diabetic retinopathy and retinitis pigmentosa, among others.

In one embodiment, the intraocular implants comprise a retinoid component and a biodegradable polymer matrix. The retinoid component is associated with a biodegradable polymer matrix that degrades at a rate effective to sustain release of an amount of the retinoid component from the implant effective to treat an ocular condition. The intraocular implant is biodegradable or bioerodible and provides a sustained release of the retinoid component in an eye for extended periods of time, such as for more than one week, for example for about one month or more and up to about six months or more.

Retinoids of the present implants may be capable of activating or enhancing the activity of an RARα, an RARβ, an RARγ, an RXRα, an RXRβ, or an RXRγ. In certain implants, the retinoid component is a hydrophilic compound, for example, the retinoid may have a log partition coefficient (log P) of less than about 3.0. In certain implants, the retinoid component is tazarotenic acid, a prodrug of tazarotenic acid, salts thereof, and mixtures thereof.

The biodegradable polymer matrix of the foregoing implants may be a mixture of biodegradable polymers or the matrix may comprise a single type of biodegradable polymer. For example, the matrix may comprise a polymer selected from the group consisting of polylactides, poly (lactide-co-glycolides), polycaprolactones, and combinations thereof.

A method of making the present implants involves combining or mixing the retinoid component with a biodegradable polymer or polymers. The mixture may then be extruded or compressed to form a single composition. The single composition may then be processed to form individual implants suitable for placement in an eye of a patient.

Other implants may comprise a therapeutic component, which comprises, consists essentially of, or consists of a retinoid component, and a drug release sustaining component which includes a non-biodegradable polymer, such as a coating with one or more orifices or holes, such as the implants disclosed in U.S. Pat. No. 6,331,313.

Additional implants may comprise a drug release sustaining component that comprises a hydrogel.

The implants may be placed in an ocular region to treat a variety of ocular conditions, such as treating, preventing, or reducing at least one symptom associated with an ocular condition, including without limitation, proliferative vitreal retinopathy, age related macular degeneration, diabetic retinopathy and retinitis pigmentosa.

Kits in accordance with the present invention may comprise one or more of the present implants, and instructions for using the implants. For example, the instructions may explain how to administer the implants to a patient, and types of conditions that may be treated with the implants.

Each and every feature described herein, and each and every combination of two or more of such features, is included within the scope of the present invention provided that the features included in such a combination are not mutually inconsistent. In addition, any feature or combination of features may be specifically excluded from any embodiment of the present invention.

Additional aspects and advantages of the present invention are set forth in the following description and claims, particularly when considered in conjunction with the accompanying drawings.

### DRAWINGS

FIG. 1A is a graph illustrating the release of tazarotene from PLA microspheres. The graph depicts three separate release studies of the same sample.

FIG. 1B is a graph illustrating the release of tazarotene from PLGA microspheres. The graph depicts three separate release studies of the same sample.

FIG. 2 is a graph illustrating in-vitro release of tazarotenic acid from poly (lactic acid), PLA, and poly (lactide-co-glycolide) implants into PBS, pH 7.4.

FIG. 3 is a graph illustrating tazarotene concentration (mean±SD) in aqueous humor, vitreous humor, and retina (N=4) after a single subconjunctival injection of 1 mg tazarotene in a suspension.

FIG. 4 is a graph illustrating tazarotenic acid concentration (mean±SD) in aqueous humor, vitreous humor, and retina (N=4) after a single subconjunctival injection of 1 mg tazarotene in a suspension.

FIG. 5 is a graph illustrating tazarotene concentration (mean±SD) in aqueous humor, vitreous humor, and retina (N=4) after a single subconjunctival injection of 1 mg tazarotene in a solution.

FIG. 6 is a graph illustrating tazarotenic acid concentration (mean±SD) in aqueous humor, vitreous humor, and retina (N=4) after a single subconjunctival injection of 1 mg tazarotene in a solution.

FIG. 7 is a graph illustrating tazarotene concentration (mean±SD) in aqueous humor, vitreous humor, and retina (N=4) after a single subconjunctival injection of 0.5 mg tazarotene in PGLA microspheres.

FIG. 8 is a graph illustrating tazarotenic acid concentration (mean±SD) in aqueous humor, vitreous humor, and retina (N=4) after a single subconjunctival injection of 0.5 mg tazarotene in PGLA microspheres.

FIG. 9 is a graph illustrating intravitreal concentrations of tazarotene and tazarotenic acid following Intravitreal administration of tazarotene.

FIG. 10 is a graph illustrating vitreous tazarotene/tazarotenic acid concentration ratios: 1. Subconjunctival Suspension, 2. Subconjunctival Oil, 3. Subconjunctival Microsphere, 4. Intravitreal Injection.

FIG. 11 is a graph illustrating tissue-to-plasma concentration ratios of after twenty-one days of daily topical application of 14C-tazarotene to the skin of rats.

FIG. 12 is a graph illustrating tazarotene retinal/vitreous ratios after intraocular and subconjunctival administration: 1. Intravitreal Injection 2. Subconjunctival Suspension, 3. Subconjunctival Oil, 4. Subconjunctival Microspheres.

FIG. 15A is a graph illustrating tazarotene release profiles—RG502H (0.5% Tween-80/Saline, 37° C., n=3).

FIG. 15B is a graph illustrating tazarotene release profiles—RG502 (0.5% Tween-80/Saline, 37° C., n=3).

FIG. 15C is a graph illustrating tazarotene release profiles—RG752 (0.5% Tween-80/Saline, 37° C., n=3).

FIG. 15D is a graph illustrating tazarotene release profiles—R202H (0.5% Tween-80/Saline 37, ° C., n=3).

FIG. 16 is a graph illustrating tazarotene release profile of formulation 1, 4, 9, 17, 18, and 19 (500 μg dose, n=3, 37° C.).

FIG. 17 is a graph illustrating tazarotene release of formulation 1, 9, 12, and 17 (500 μg dose, n=3, 37° C.).

FIG. 18 is a graph illustrating tazarotene release profile of GLP Lot #229-01 (500 μg dose, 37° C., n=6).

FIG. 19 is a graph illustrating tazarotene release profiles—50 μg dose (0.5% Tween-80/Saline, 37° C., n=6).

FIG. 20 is a graph illustrating tazarotene release profiles—50 μg dose (0.5% Tween-80/Saline, 37° C., n=6).

FIG. 21 is a graph illustrating tazarotene release profile—50 μg dose (0.5% Tween-80/Saline, 37° C., n=6).

FIG. 22 is a graph illustrating tazarotene release profile (0.5% Tween-80/Saline, 37° C., n=3).

FIG. 23A is a graph illustrating tazarotene release profiles with polymer blend (0.5% Tween-80/Saline, 37° C., n=6).

FIG. 23B is a graph illustrating tazarotene release profiles with polymer blend (0.5% Tween-80/Saline, 37° C., n=6).

FIG. 24 is a graph illustrating tazarotene wafer release profiles (0.5% Tween-80/Saline, 37° C., n=6).

FIG. 25 is a graph illustrating tazarotene release profile from microspheres.

FIG. 26 is a graph illustrating tazarotenic acid concentration (mean±SEM) in aqueous humor (upper graph) and lens (lower graph) (N=4) after a single intravitreal implantation of Formulation #1, #9, and #12 containing 500 μg tazarotene.

FIG. 27 is a graph illustrating tazarotenic acid concentration (mean±SEM) in retina (upper graph) and vitreous humor (lower graph) (N=4) after a single intravitreal implantation of Formulation #1, #9, and #12 containing 500 μg tazarotene.

FIG. 28 is a graph illustrating tazarotenic acid concentration (mean±SEM) in plasma (N=2) after a single intravitreal implantation of Formulation #1, #9, and #12 containing 500 μg tazarotene.

FIG. 29 is a graph of tazarotenic acid and tazarotene concentrations in the vitreous humor, retina, and plasma from tazarotene intravitreal implants.

FIG. 30 is a graph of tazarotenic acid and tazarotene concentrations in the vitreous humor, retina, and plasma from tazarotene subconjunctival implants.

FIG. 31 is a graph of Fastenberg results from PVR implants.

### DESCRIPTION

As described herein, controlled and sustained administration of a therapeutic agent through the use of one or more intraocular implants may improve treatment of undesirable ocular conditions. The implants comprise a pharmaceutically acceptable polymeric composition and are formulated to release one or more pharmaceutically active agents, such as retinoids, such as RAR or RXR agonists, or retinoid precursors, over an extended period of time. The implants are effective to provide a therapeutically effective dosage of the agent or agents directly to a region of the eye to treat, prevent, and/or reduce one or more symptoms of one or more undesirable ocular conditions. Thus, with a single administration, therapeutic agents will be made available at the site where they are needed and will be maintained for an extended period of time, rather than subjecting the patient to repeated injections or, in the case of self-administered drops, ineffective treatment with only limited bursts of exposure to the active agent or agents or, in the case of systemic administration, higher systemic exposure and concomitant side effects or, in the case of non-sustained release dosages, potentially toxic transient high tissue concentrations associated with pulsed, non-sustained release dosing.

An intraocular implant in accordance with the disclosure herein comprises a therapeutic component and a drug release sustaining component associated with the therapeutic component. In accordance with the present invention, the therapeutic component comprises, consists essentially of, or consists of, a retinoid component, such as a RAR agonist or a RXR agonist, or a RAR agonist precursor or prodrug, or a RXR agonist precursor or prodrug. The drug release sustaining component is associated with the therapeutic component to sustain release of an effective amount of the therapeutic component into an eye in which the implant is placed. The amount of the therapeutic component is released into the eye for a period of time greater than about one week after the implant is placed in the eye, and is effective in treating and/or reducing at least one symptom of one or more ocular conditions, such as proliferative vitreal retinopathy, age related macular degeneration, diabetic retinopathy and retinitis pigmentosa, and the like.

### DEFINITIONS

For the purposes of this description, we use the following terms as defined in this section, unless the context of the word indicates a different meaning.

As used herein, an “intraocular implant” refers to a device or element that is structured, sized, or otherwise configured to be placed in an eye. Intraocular implants are generally biocompatible with physiological conditions of an eye and do not cause unacceptable adverse side effects. Intraocular implants may be placed in an eye without disrupting vision of the eye.

As used herein, a “therapeutic component” refers to a portion of an intraocular implant comprising one or more therapeutic agents or substances used to treat a medical condition of the eye. The therapeutic component may be a discrete region of an intraocular implant, or it may be homogenously distributed throughout the implant. The therapeutic agents of the therapeutic component are typically ophthalmically acceptable, and are provided in a form that does not cause adverse reactions when the implant is placed in an eye.

As used herein, a “drug release sustaining component” refers to a portion of the intraocular implant that is effective to provide a sustained release of the therapeutic agents of the implant. A drug release sustaining component may be a biodegradable polymer matrix, or it may be a coating covering a core region of the implant that comprises a therapeutic component.

As used herein, “associated with” means mixed with, dispersed within, coupled to, covering, or surrounding.

As used herein, an “ocular region” or “ocular site” refers generally to any area of the eyeball, including the anterior and posterior segment of the eye, and which generally includes, but is not limited to, any functional (e.g., for vision) or structural tissues found in the eyeball, or tissues or cellular layers that partly or completely line the interior or exterior of the eyeball. Specific examples of areas of the eyeball in an ocular region include the anterior chamber, the posterior chamber, the vitreous cavity, the choroid, the suprachoroidal space, the conjunctiva, the subconjunctival space, the episcleral space, the intracorneal space, the epicorneal space, the sclera, the pars plana, surgically-induced avascular regions, the macula, and the retina.

As used herein, an “ocular condition” is a disease, ailment or condition which affects or involves the eye or one of the parts or regions of the eye. Broadly speaking the eye includes the eyeball and the tissues and fluids which constitute the eyeball, the periocular muscles (such as the oblique and rectus muscles) and the portion of the optic nerve which is within or adjacent to the eyeball.

An anterior ocular condition is a disease, ailment or condition which affects or which involves an anterior (i.e. front of the eye) ocular region or site, such as a periocular muscle, an eye lid or an eye ball tissue or fluid which is located anterior to the posterior wall of the lens capsule or ciliary muscles. Thus, an anterior ocular condition primarily affects or involves the conjunctiva, the cornea, the anterior chamber, the iris, the posterior chamber (behind the iris but in front of the posterior wall of the lens capsule), the lens or the lens capsule and blood vessels and nerve which vascularize or innervate an anterior ocular region or site.

Thus, an anterior ocular condition can include a disease, ailment or condition, such as for example, aphakia; pseudophakia; astigmatism; blepharospasm; cataract; conjunctival diseases; conjunctivitis; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

A posterior ocular condition is a disease, ailment or condition which primarily affects or involves a posterior ocular region or site such as choroid or sclera (in a position posterior to a plane through the posterior wall of the lens capsule), vitreous, vitreous chamber, retina, retinal pigmented epithelium, Bruch's membrane, optic nerve (i.e. the optic disc), and blood vessels and nerves which vascularize or innervate a posterior ocular region or site.

Thus, a posterior ocular condition can include a disease, ailment or condition, such as for example, acute macular neuroretinopathy; Behcet's disease; choroidal neovascularization; diabetic uveitis; histoplasmosis; infections, such as fungal or viral-caused infections; macular degeneration, such as acute macular degeneration, non-exudative age related macular degeneration and exudative age related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic ophthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, and glaucoma. Glaucoma can be considered a posterior ocular condition because the therapeutic goal is to prevent the loss of or reduce the occurrence of loss of vision due to damage to or loss of retinal cells or optic nerve cells (i.e. neuroprotection).

The term “biodegradable polymer” refers to a polymer or polymers which degrade in vivo, and wherein erosion of the polymer or polymers over time occurs concurrent with or subsequent to release of the therapeutic agent. Specifically, hydrogels such as methylcellulose which act to release drug through polymer swelling are specifically excluded from the term “biodegradable polymer”. The terms “biodegradable” and “bioerodible” are equivalent and are used interchangeably herein. A biodegradable polymer may be a homopolymer, a copolymer, or a polymer comprising more than two different polymeric units.

The term “treat”, “treating”, or “treatment” as used herein, refers to reduction or resolution or prevention of an ocular condition, ocular injury or damage, or to promote healing of injured or damaged ocular tissue.

The term “therapeutically effective amount” as used herein, refers to the level or amount of agent needed to treat an ocular condition, or reduce or prevent ocular injury or damage without causing significant negative or adverse side effects to the eye or a region of the eye.

Intraocular implants have been developed which can release drug loads over various time periods. These implants, which when inserted into an eye, such as the vitreous of an eye, provide therapeutic levels of a retinoid component, such as a RAR or RXR agonist, or precursor thereof, for extended periods of time (e.g., for about 1 week or more). The disclosed implants are effective in treating ocular conditions, such as proliferative vitreal retinopathy, age related macular degeneration, diabetic retinopathy and retinitis pigmentosa.

In one embodiment of the present invention, an intraocular implant comprises a biodegradable polymer matrix. The biodegradable polymer matrix is one type of a drug release sustaining component. The biodegradable polymer matrix is effective in forming a biodegradable intraocular implant. The biodegradable intraocular implant comprises a retinoid component associated with the biodegradable polymer matrix. The matrix degrades at a rate effective to sustain release of an amount of the retinoid component for a time greater than about one week from the time in which the implant is placed in ocular region or ocular site, such as the vitreous of an eye.

The retinoid component preferably includes an active retinoid agent and/or a precursor of an active retinoid agent effective to selectively, and even specifically, affect, for example, bind to and/or activate and/or inhibit the activation of and/or block, at least one of RAR-beta and RAR-gamma relative to RAR-alpha.

As used herein, the terms “selectively” or “more selectively” refer to the ability of an active retinoid agent to affect one or more first subtype(s) of RAR relative to one or more other second subtype(s) of RAR. In preferred embodiments, the first subtype(s) is affected at least about 5, about 10, about 20, about 50, about 100, or about 1000 times more than the second subtype(s). The term “specifically” refers to the ability of an active retinoid agent to affect one or more first RAR subtype(s) without substantially affecting, or preferably without affecting in a detectable way, one or more other second RAR subtype(s).

In certain implants, the retinoid component includes an active retinoid agent or a precursor of an active retinoid agent effective to selectively or even specifically affect both RAR-beta and RAR-gamma relative to RAR-alpha. The retinoid component advantageously includes an active retinoid agent or a precursor of an active retinoid agent effective to selectively or even specifically activate or inhibit the activation of or block at least one or both of RAR-beta and RAR-gamma relative to RAR-alpha. In one embodiment, the retinoid component includes an active retinoid agent or a precursor of an active retinoid agent effective to selectively or even specifically activate at least one of or both RAR-beta and RAR-gamma relative to RAR-alpha.

Although the present implants may comprise a large variety of retinoid components, such as active retinoid agents or precursors of active retinoid agents which have RAR-antagonist activity and RAR-inverse agonist activity, the present invention is particularly useful with retinoid components which include active retinoid agents or precursors of active retinoid agents which have RAR-agonist activity.

In certain implants, the retinoid component includes an active retinoid agent having a substantial degree of water solubility, for example, is more water soluble than isotretinoin, or is metabolically converted in the human or animal into an active retinoid agent having a substantial degree of water solubility. In this way, it is possible to avoid having the retinoid cross lipid barriers, such as the blood brain barrier and the retinal-blood barrier. This specifically avoids some of the usual adverse side effects of other retinoids, such as central nervous system (CNS) effects and eye toxicities.

The retinoid component may comprise an active RAR ligand which is substantially ineffective to bind to or activate or block RXRs and/or a precursor of an active RAR ligand substantially ineffective to bind to or activate or block RXRs.

Among the retinoid components useful in the present invention include the following compounds of formula I

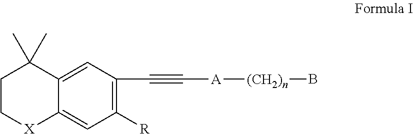

wherein X is S, O, or NR═ where R═ is hydrogen or lower alkyl; R is hydrogen or lower alkyl; A is pyridinyl, thienyl, furyl, pyridazinyl, pyrimidinyl or pyrazinyl; n is 0-2; and B is H, —COOH or a pharmaceutically acceptable salt, ester or amide thereof, —CH₂OH or an ether or ester derivative, or —CHO or an acetal derivative, or —COR₁ or a ketal derivative where R₁ is —(—CH₂)ₘCH₃ where m is 0-4.

The compounds of formula I can be made by reacting a compound of formula II with a compound of formula III in the presence of cuprous iodide and Pd(PQ₃)₂Cl₂ or a similar complex. Compounds of formula II and formula III are as follows:

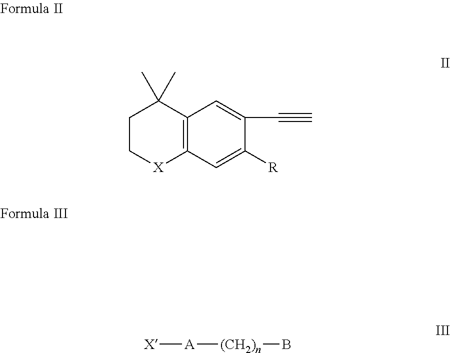

where X═ is a halogen, preferably I; n and A are the same as defined above; and B is H, or a protected acid, alcohol, aldehyde or ketone, giving the corresponding compound of formula I.

Alternately, the compounds of formula I can be made by reacting a zinc salt of formula IV with a compound of formula III in the presence of Pd(PQ₃)₄ (Q is phenyl) or a similar complex,

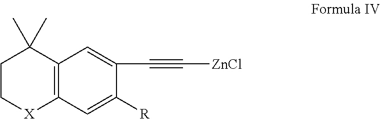

giving the corresponding compound of formula I.

Further, the compounds of formula I can be made by homologating a compound of formula V

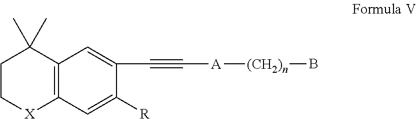

where

n is 0-1 to give an acid of formula I; or

converting an acid of formula I to a salt; or

forming an acid addition salt;

converting an acid of formula I to an ester; or

converting an acid of formula I to an amide; or

reducing an acid of formula I to an alcohol or aldehyde; or

converting an alcohol of formula I to an ether or ester; or

oxidizing an alcohol of formula I to an aldehyde; or

converting an aldehyde of formula Ito an acetal; or

converting a ketone of formula I to a ketal.

The term “ester” as used here refers to and covers any compound falling within the definition of that term as classically used in organic chemistry. Where A is —COOH, this term covers the products derived from treatment of this function with alcohols. Where the ester is derived from compounds where A is —CH₂OH, this term covers compounds of the formula —CH₂—OOCR— where R is any substituted or unsubstituted aliphatic, aromatic or aliphatic-aromatic group.

Preferred esters are derived from the saturated aliphatic alcohols or acids of about 10 or fewer carbon atoms or the cyclic or saturated aliphatic cyclic alcohols and acids of about 5 to about 10 carbon atoms. Particularly preferred aliphatic esters are those derived from lower alkyl acids and alcohols. Here, and where ever else used, lower alkyl means having 1 to about 6 carbon atoms. Also preferred are the phenyl or lower alkylphenyl esters.

Amide has the meaning classically accorded that term in organic chemistry. In this instance, it includes the unsubstituted amides and all aliphatic and aromatic mono- and di-substituted amides. Preferred amides are the mono- and di-substituted amides derived from the saturated aliphatic radicals of about 10 or fewer carbon atoms or the cyclic or saturated aliphatic-cyclic radicals of about 5 to about 10 carbon atoms. Particularly preferred amides are those derived from lower alkyl amines. Also preferred are mono- and di-substituted amides derived from the phenyl or lower alkylphenyl amines. Unsubstituted amides are also preferred.

Acetals and ketals include the radicals of the formula —CK where K is (—OR)₂. Here, R is lower alkyl. K may also be —OR₁O— where R₁ is lower alkyl of about 2 to about 5 carbon atoms, straight chain or branched.

Certain retinoid components for use in the present implants include those where the ethynyl group and the B group are attached to the 2 and 5 positions respectively of a pyridine ring (the 6 and 3 positions in the nicotinic acid nomenclature being equivalent to the 2/5 designation in the pyridine nomenclature) or the 5 and 2 positions respectively of a thiophene group respectively; n is 0; and B is —COOH, an alkali metal salt or organic amine salt, or a lower alkyl ester, or —CH₂OH and the lower alkyl esters and ethers thereof, or —CHO and acetal derivatives thereof.

More preferred compounds for use in the present implants include:
ethyl 6-(2-(4,4-dimethylthiochroman-6-yl)ethynyl)-nicotinate;
6-(2-(4,4-dimethylthiochroman-6-yl)ethynyl)nicotinic acid;
6-(2-(4,4-dimethylchroman-6-yl)ethynyl)nicotinic acid;
ethyl 6-(2-(4,4-dimethylchroman-6-yl)ethynyl)nicotinate;
ethyl 6-(2-(4,4,7-trimethylthiochroman-6-yl)ethynyl)-nicotinate;
ethyl 6-(2-(4,4-dimethyl-1,2,3,4-tetrahydroquinolin-6-yl)ethynyl)nicotinate;
ethyl 5-(2-(4,4-dimethylthiochroman-6-yl)ethynyl)-thiophene-2-carboxylate;
6-(2-(4,4-dimethylthiochroman-6-yl)ethynyl)-3-pyridylmethanol; and
2-(2-(4,4-dimethylthiochroman-6-yl)ethynyl)-5-pyridinecarboxaldehyde.

These compounds, and methods of making these compounds are described in U.S. Pat. No. 5,089,509.

A class of useful retinoid components has the structure:

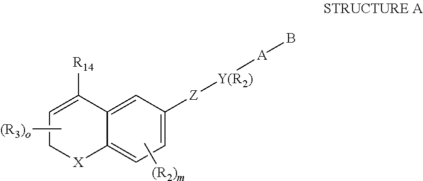

wherein X is S, O, NR′ where R′ is H or alkyl of 1 to 6 carbons, or

X is [C(R₁)₂]ₙ where R₁ is independently H or alkyl of 1 to 6 carbons, and n is an integer between, and including, 0 and 2, and;

R₂ is hydrogen, lower alkyl of 1 to 6 carbons, F, Cl, Br, I, CF₃, fluoro substituted alkyl of 1 to 6 carbons, OH, SH, alkoxy of 1 to 6 carbons, or alkylthio of 1 to 6 carbons, and;

R₃ is hydrogen, lower alkyl of 1 to 6 carbons or F, and;

m is an integer having the value of 0-3, and;

o is an integer having the value of 0-3, and;
Z is —C≡C—,
—N═N—,
—N═CR₁—,
—CR₁═N,
—(CR₁═CR₁)_{n′}— where n′ is an integer having the value 0-5,
—CO—NR₁—,
—CS—NR₁—,
—NR₁—CO,
—NR₁—CS,
—COO—,
—OCO—;
—CSO—;
—OCS—;

Y is a phenyl or naphthyl group, or heteroaryl selected from a group consisting of pyridyl, thienyl, furyl, pyridazinyl, pyrimidinyl, pyrazinyl, thiazolyl, oxazolyl, imidazolyl and pyrrazolyl, said phenyl and heteroaryl groups being optionally substituted with one or two R₂ groups, or

when Z is —(CR₁═CR₁)_{n′}— and n′ is 3, 4 or 5 then Y represents a direct valence bond between said (CR₂═CR₂)_{n′} group and B;

A is (CH₂)_{q} where q is 0-5, lower branched chain alkyl having 3-6 carbons, cycloalkyl having 3-6 carbons, alkenyl having 2-6 carbons and 1 or 2 double bonds, alkynyl having 2-6 carbons and 1 or 2 triple bonds;

B is hydrogen, COOH or a pharmaceutically acceptable salt thereof, COOR₈, CONR₉R₁₀, —CH₂OH, CH₂OR₁₁, CH₂OCOR₁₁, CHO, CH(OR₁₂)₂, CHOR₁₃O, —COR₇, CR₇(OR₁₂)₂, CR₇OR₁₃O, or tri-lower alkylsilyl, where R₇ is an alkyl, cycloalkyl or alkenyl group containing 1 to 5 carbons, R₈ is an alkyl group of 1 to 10 carbons or trimethylsilylalkyl where the alkyl group has 1 to 10 carbons, or a cycloalkyl group of 5 to 10 carbons, or R₈ is phenyl or lower alkylphenyl, R₉ and R₁₀ independently are hydrogen, an alkyl group of 1 to 10 carbons, or a cycloalkyl group of 5-10 carbons, or phenyl or lower alkylphenyl, R₁₁ is lower alkyl, phenyl or lower alkylphenyl, R₁₂ is lower alkyl, and R₁₃ is divalent alkyl radical of 2-5 carbons, and

R₁₄ is (R₁₅)ᵣ-phenyl, (R₁₈)ᵣ-naphthyl, or (R₁₅)ᵣ— heteroaryl where the heteroaryl group has 1 to 3 heteroatoms selected from the group consisting of O, S and N, r is an integer having the values of 0-5, and

R₁₅ is independently H, F, Cl, Br, I, NO₂, N(R₈)₂, N(R₈)COR₅, NR₈CON(R₈)₂, OH, OCOR₈, OR₈, CN, an alkyl group having 1 to 10 carbons, fluoro substituted alkyl group having 1 to 10 carbons, an alkenyl group having 1 to 10 carbons and 1 to 3 double bonds, alkynyl group having 1 to 10 carbons and 1 to 3 triple bonds, or a trialkylsilyl or trialkylsilyloxy group where the alkyl groups independently have 1 to 6 carbons.

Such compounds can be made using well known techniques. For example, see U.S. Pat. No. 5,776,699.

One particularly useful class of retinoid components for use in the present invention is selected from active acetylenic retinoid agents, precursors of active acetylenic retinoid agents and mixtures thereof. Active acetylenic retinoid agents includes active retinoid agents including at least one — C̅C— group. Examples of such retinoid components are set forth elsewhere herein.

Especially useful retinoid components useful in the present methods include tazarotene, tazarotenic acid and mixtures thereof. Particularly effective results are obtained when tazarotene is employed as the retinoid component.

These implants may also include salts of the retinoid component. Pharmaceutically acceptable acid addition salts of the compounds of the invention are those formed from acids which form non-toxic addition salts containing pharmaceutically acceptable anions, such as the hydrochloride, hydrobromide, hydroiodide, sulfate, or bisulfate, phosphate or acid phosphate, acetate, maleate, fumarate, oxalate, lactate, tartrate, citrate, gluconate, saccharate and p-toluene sulfonate salts.

Thus, the implant may comprise a therapeutic component which comprises, consists essentially of, or consists of, a retinoid, such as tazarotenic acid, tazarotenic acid precursors, salts thereof, and mixtures thereof. These elements of the therapeutic component may be understood to be a retinoid component. The biodegradable polymer matrix of such implants may be substantially free of polyvinyl alcohol, or in other words, includes no polyvinyl alcohol.

Additional retinoid components may be obtained using conventional methods, such as by routine chemical synthesis methods known to persons of ordinary skill in the art. Some examples of structures and methods of making retinoid components, are provided in U.S. Pat. No. 5,776,699, U.S. Pat. No. 5,958,954, U.S. Pat. No. 5,877,207, and U.S. Pat. No. 5,919,970.

Therapeutically effective retinoid components may be screened and identified using conventional screening technologies. In a broad sense, any compound can be tested for RAR activity, for example, using conventional and well known techniques, for example, without limitation, those described in the above-noted patents.

Once a compound has been determined to have suitable RAR activity, it can be administered to a test animal with appropriate monitoring for side effects. Comparing the results of such monitoring with similar monitoring of test animals given reference retinoid agents allows one to determine if the compound is useful in accordance with the present invention.

In other aspects of the present invention, one or more compounds, for example, from a screening library of compounds, which are known to have or have been tested, using conventional and well known techniques, and found to have useful RAR activity, can be individually or collectively tested for RXR activity using conventional and well known testing procedures (see, for example, U.S. Pat. No. 5,906,920).

Compounds with substantially no RXR activity may be selected for further testing. Compounds with desired RAR activity and substantially no RXR activity are useful in accordance with one or more aspects of the present invention.

Other well known and straightforward test methods and/or assays may be employed to determine the selectivity or specificity of an RAR active compound to, RAR-alpha, RAR-beta and RAR-gamma. For example, using conventional and well known assays, for example, such as set forth in U.S. Pat. No. 5,776,699, and/or the above-noted patents, the selectively or specificity of a compound to RAR-alpha, RAR-beta and RAR-gamma can be determined. Based on the results of such assays, one can determine whether or not a compound is useful in accordance with one or more aspects of the present invention.

Further confirmation that any compound is useful in accordance with the present invention can be obtained by orally administering the compound to an animal and monitoring the presence or absence of side effects.

In any event, determining which compounds are useful in accordance with the present invention can be accomplished using conventional and well known techniques, without undue experimentation.

The retinoid component may be in a particulate or powder form and entrapped by the biodegradable polymer matrix. Usually, retinoid component particles in intraocular implants will have an effective average size less than about 3000 nanometers. In certain implants, the particles may have an effective average particle size about an order of magnitude smaller than 3000 nanometers. For example, the particles may have an effective average particle size of less than about 500 nanometers. In additional implants, the particles may have an effective average particle size of less than about 400 nanometers, and in still further embodiments, a size less than about 200 nanometers.

The retinoid component of the implant is preferably from about 10% to 90% by weight of the implant. More preferably, the retinoid component is from about 20% to about 80% by weight of the implant. In a preferred embodiment, the retinoid component comprises about 40% by weight of the implant (e.g., 30%-50%). In another embodiment, the retinoid component comprises about 60% by weight of the implant.

Suitable polymeric materials or compositions for use in the implant include those materials which are compatible, that is biocompatible, with the eye so as to cause no substantial interference with the functioning or physiology of the eye. Such materials preferably are at least partially and more preferably substantially completely biodegradable or bioerodible.

Examples of useful polymeric materials include, without limitation, such materials derived from and/or including organic esters and organic ethers, which when degraded result in physiologically acceptable degradation products, including the monomers. Also, polymeric materials derived from and/or including, anhydrides, amides, orthoesters and the like, by themselves or in combination with other monomers, may also find use. The polymeric materials may be addition or condensation polymers, advantageously condensation polymers. The polymeric materials may be cross-linked or non-cross-linked, for example not more than lightly cross-linked, such as less than about 5%, or less than about 1% of the polymeric material being cross-linked. For the most part, besides carbon and hydrogen, the polymers will include at least one of oxygen and nitrogen, advantageously oxygen. The oxygen may be present as oxy, e.g. hydroxy or ether, carbonyl, e.g. non-oxo-carbonyl, such as carboxylic acid ester, and the like. The nitrogen may be present as amide, cyano and amino. The polymers set forth in Heller, Biodegradable Polymers in Controlled Drug Delivery, In: CRC Critical Reviews in Therapeutic Drug Carrier Systems, Vol. 1, CRC Press, Boca Raton, Fla. 1987, pp 39-90, which describes encapsulation for controlled drug delivery, may find use in the present implants.

Of additional interest are polymers of hydroxyaliphatic carboxylic acids, either homopolymers or copolymers, and polysaccharides. Polyesters of interest include polymers of D-lactic acid, L-lactic acid, racemic lactic acid, glycolic acid, polycaprolactone, and combinations thereof. Generally, by employing the L-lactate or D-lactate, a slowly eroding polymer or polymeric material is achieved, while erosion is substantially enhanced with the lactate racemate.

Among the useful polysaccharides are, without limitation, calcium alginate, and functionalized celluloses, particularly carboxymethylcellulose esters characterized by being water insoluble, a molecular weight of about 5 kD to 500 kD, for example.

Other polymers of interest include, without limitation, polyesters, polyethers and combinations thereof which are biocompatible and may be biodegradable and/or bioerodible.

Some preferred characteristics of the polymers or polymeric materials for use in the present invention may include biocompatibility, compatibility with the therapeutic component, ease of use of the polymer in making the drug delivery systems of the present invention, a half-life in the physiological environment of at least about 6 hours, preferably greater than about one day, not significantly increasing the viscosity of the vitreous, and water insolubility.

The biodegradable polymeric materials which are included to form the matrix are desirably subject to enzymatic or hydrolytic instability. Water soluble polymers may be cross-linked with hydrolytic or biodegradable unstable cross-links to provide useful water insoluble polymers. The degree of stability can be varied widely, depending upon the choice of monomer, whether a homopolymer or copolymer is employed, employing mixtures of polymers, and whether the polymer includes terminal acid groups.

Equally important to controlling the biodegradation of the polymer and hence the extended release profile of the implant is the relative average molecular weight of the polymeric composition employed in the implant. Different molecular weights of the same or different polymeric compositions may be included in the implant to modulate the release profile. In certain implants, the relative average molecular weight of the polymer will range from about 9 to about 64 kD, usually from about 10 to about 54 kD, and more usually from about 12 to about 45 kD.

In some implants, copolymers of glycolic acid and lactic acid are used, where the rate of biodegradation is controlled by the ratio of glycolic acid to lactic acid. The most rapidly degraded copolymer has roughly equal amounts of glycolic acid and lactic acid. Homopolymers, or copolymers having ratios other than equal, are more resistant to degradation. The ratio of glycolic acid to lactic acid will also affect the brittleness of the implant, where a more flexible implant is desirable for larger geometries. The % of polylactic acid in the polylactic acid polyglycolic acid (PLGA) copolymer can be 0-100%, preferably about 15-85%, more preferably about 35-65%. In some implants, a 50/50 PLGA copolymer is used.

The biodegradable polymer matrix of the intraocular implant may comprise a mixture of two or more biodegradable polymers. For example, the implant may comprise a mixture of a first biodegradable polymer and a different second biodegradable polymer. One or more of the biodegradable polymers may have terminal acid groups.

Release of a drug from an erodible polymer is the consequence of several mechanisms or combinations of mechanisms. Some of these mechanisms include desorption from the implants surface, dissolution, diffusion through porous channels of the hydrated polymer and erosion. Erosion can be bulk or surface or a combination of both. As discussed herein, the matrix of the intraocular implant may release drug at a rate effective to sustain release of an amount of the retinoid component for more than one week after implantation into an eye. In certain implants, therapeutic amounts of the retinoid component are released for more than about one month, and even for about six months or more.

One example of the biodegradable intraocular implant comprises tazarotene, tazarotenic acid, or a combination thereof with a biodegradable polymer matrix that comprises a poly (lactide-co-glycolide) or a poly (D,L-lactide-co-glycolide). The implant may have an amount of the retinoid component from about 40% to about 70% by weight of the implant. Such a mixture is effective in sustaining release of a therapeutically effective amount of the retinoid component for a time period from about one month to about four months from the time the implant is placed in an eye.

The release of the retinoid component from the intraocular implant comprising a biodegradable polymer matrix may include an initial burst of release followed by a gradual increase in the amount of the retinoid component released, or the release may include an initial delay in release of the retinoid component followed by an increase in release. When the implant is substantially completely degraded, the percent of the retinoid component that has been released is about one hundred. Compared to existing implants, the implants disclosed herein do not completely release, or release about 100% of the retinoid component, until after about one week of being placed in an eye.

It may be desirable to provide a relatively constant rate of release of the retinoid component from the implant over the life of the implant. For example, it may be desirable for the retinoid component to be released in amounts from about 0.01 μg to about 2 μg per day for the life of the implant. However, the release rate may change to either increase or decrease depending on the formulation of the biodegradable polymer matrix. In addition, the release profile of the retinoid component may include one or more linear portions and/or, one or more non-linear portions. Preferably, the release rate is greater than zero once the implant has begun to degrade or erode.

The implants may be monolithic, i.e. having the active agent or agents homogenously distributed through the polymeric matrix, or encapsulated, where a reservoir of active agent is encapsulated by the polymeric matrix. Due to ease of manufacture, monolithic implants are usually preferred over encapsulated forms. However, the greater control afforded by the encapsulated, reservoir-type implant may be of benefit in some circumstances, where the therapeutic level of the drug falls within a narrow window. In addition, the therapeutic component, including the retinoid component, may be distributed in a non-homogenous pattern in the matrix. For example, the implant may include a portion that has a greater concentration of the retinoid component relative to a second portion of the implant.

The intraocular implants disclosed herein may have a size of between about 5 μm and about 2 mm, or between about 10 μm and about 1 mm for administration with a needle, greater than 1 mm, or greater than 2 mm, such as 3 mm or up to 10 mm, for administration by surgical implantation. The vitreous chamber in humans is able to accommodate relatively large implants of varying geometries, having lengths of, for example, 1 to 10 mm. The implant may be a cylindrical pellet (e.g., rod) with dimensions of about 2 mm×0.75 mm diameter. Or the implant may be a cylindrical pellet with a length of about 7 mm to about 10 mm, and a diameter of about 0.75 mm to about 1.5 mm.

The implants may also be at least somewhat flexible so as to facilitate both insertion of the implant in the eye, such as in the vitreous, and accommodation of the implant. The total weight of the implant is usually about 250-5000 μg, more preferably about 500-1000 μg. For example, an implant may be about 500 μg, or about 1000 μg. For non-human individuals, the dimensions and total weight of the implant(s) may be larger or smaller, depending on the type of individual. For example, humans have a vitreous volume of approximately 3.8 ml, compared with approximately 30 ml for horses, and approximately 60-100 ml for elephants. An implant sized for use in a human may be scaled up or down accordingly for other animals, for example, about 8 times larger for an implant for a horse, or about, for example, 26 times larger for an implant for an elephant.

Thus, implants can be prepared where the center may be of one material and the surface may have one or more layers of the same or a different composition, where the layers may be cross-linked, or of a different molecular weight, different density or porosity, or the like. For example, where it is desirable to quickly release an initial bolus of drug, the center may be a polylactate coated with a polylactate polyglyconate copolymer, so as to enhance the rate of initial degradation. Alternatively, the center may be polyvinyl alcohol coated with polylactate, so that upon degradation of the polylactate exterior the center would dissolve and be rapidly washed out of the eye.

The implants may be of any geometry including fibers, sheets, films, microspheres, spheres, circular discs, plaques and the like. The upper limit for the implant size will be determined by factors such as toleration for the implant, size limitations on insertion, ease of handling, etc. Where sheets or films are employed, the sheets or films will be in the range of at least about 0.5 mm×0.5 mm, usually about 3-10 mm×5-10 mm with a thickness of about 0.1-1.0 mm for ease of handling. Where fibers are employed, the fiber diameter will generally be in the range of about 0.05 to 3 mm and the fiber length will generally be in the range of about 0.5-10 mm. Spheres may be in the range of about 0.5 μm to 4 mm in diameter, with comparable volumes for other shaped particles.

The size and form of the implant can also be used to control the rate of release, period of treatment, and drug concentration at the site of implantation. Larger implants will deliver a proportionately larger dose, but depending on the surface to mass ratio, may have a slower release rate. The particular size and geometry of the implant are chosen to suit the site of implantation.

The implants may be provided in kits, such as sealed packages and the like. The implants may be sterilized or non-sterilized. Advantageously, the present implants remain stable for relatively long periods of time such as six months or more in either sterile or non-sterile settings. For example, the present implants retain their physical appearance and release profiles of the therapeutic component, such as the retinoid component for at least 6 months, and even for at least a year under temperature ranges from about twenty degrees Celsius to about forty degrees Celsius. Thus, the implants may be stored for substantial periods of time without significant loss of therapeutic efficacy.

The proportions of retinoid component, polymer, and any other modifiers may be empirically determined by formulating several implants with varying proportions. A USP approved method for dissolution or release test can be used to measure the rate of release (USP 23; NF 18 (1995) pp. 1790-1798). For example, using the infinite sink method, a weighed sample of the implant is added to a measured volume of a solution containing 0.9% NaCl in water, where the solution volume will be such that the drug concentration is after release is less than 5% of saturation. The mixture is maintained at 37° C. and stirred slowly to maintain the implants in suspension. The appearance of the dissolved drug as a function of time may be followed by various methods known in the art, such as spectrophotometrically, HPLC, mass spectroscopy, etc. until the absorbance becomes constant or until greater than 90% of the drug has been released.

In addition to the retinoid component included in the intraocular implants disclosed herein, the intraocular implants may also include one or more additional ophthalmically acceptable therapeutic agents. For example, the implant may include one or more antihistamines, one or more antibiotics, one or more beta blockers, one or more steroids, one or more antineoplastic agents, one or more immunosuppressive agents, one or more antiviral agents, one or more antioxidant agents, and mixtures thereof.

Pharmacologic or therapeutic agents which may find use in the present systems, include, without limitation, those disclosed in U.S. Pat. Nos. 4,474,451, columns 4-6 and 4,327,725, columns 7-8.

Examples of antihistamines include, and are not limited to, loratadine, hydroxyzine, diphenhydramine, chlorpheniramine, brompheniramine, cyproheptadine, terfenadine, clemastine, triprolidine, carbinoxamine, diphenylpyraline, phenindamine, azatadine, tripelennamine, dexchlorpheniramine, dexbrompheniramine, methdilazine, and trimeprazine doxylamine, pheniramine, pyrilamine, chlorcyclizine, thonzylamine, and derivatives thereof.

Examples of antibiotics include without limitation, cefazolin, cephradine, cefaclor, cephapirin, ceftizoxime, cefoperazone, cefotetan, cefuroxime, cefotaxime, cefadroxil, ceftazidime, cephalexin, cephalothin, cefamandole, cefoxitin, cefonicid, ceforanide, ceftriaxone, cefadroxil, cephradine, cefuroxime, cyclosporine, ampicillin, amoxicillin, cyclacillin, ampicillin, penicillin G, penicillin V potassium, piperacillin, oxacillin, bacampicillin, cloxacillin, ticarcillin, azlocillin, carbenicillin, methicillin, nafcillin, erythromycin, tetracycline, doxycycline, minocycline, aztreonam, chloramphenicol, ciprofloxacin hydrochloride, clindamycin, metronidazole, gentamicin, lincomycin, tobramycin, vancomycin, polymyxin B sulfate, colistimethate, colistin, azithromycin, augmentin, sulfamethoxazole, trimethoprim, gatifloxacin, ofloxacin, and derivatives thereof.

Examples of beta blockers include acebutolol, atenolol, labetalol, metoprolol, propranolol, timolol, and derivatives thereof.

Examples of steroids include corticosteroids, such as cortisone, prednisolone, fluorometholone, dexamethasone, medrysone, loteprednol, fluazacort, hydrocortisone, prednisone, betamethasone, prednisone, methylprednisolone, triamcinolone hexacatonide, paramethasone acetate, diflorasone, fluocinonide, fluocinolone, triamcinolone, derivatives thereof, and mixtures thereof.

Examples of antineoplastic agents include adriamycin, cyclophosphamide, actinomycin, bleomycin daunorubicin, doxorubicin, epirubicin, mitomycin, methotrexate, fluorouracil, carboplatin, carmustine (BCNU), methyl-CCNU, cisplatin, etoposide, interferons, camptothecin and derivatives thereof, phenesterine, taxol and derivatives thereof, taxotere and derivatives thereof, vinblastine, vincristine, tamoxifen, etoposide, piposulfan, cyclophosphamide, and flutamide, and derivatives thereof.

Examples of immunosuppressive agents include cyclosporine, azathioprine, tacrolimus, and derivatives thereof.

Examples of antiviral agents include interferon gamma, zidovudine, amantadine hydrochloride, ribavirin, acyclovir, valaciclovir, dideoxycytidine, phosphonoformic acid, ganciclovir and derivatives thereof.

Examples of antioxidant agents include ascorbate, alpha-tocopherol, mannitol, reduced glutathione, various carotenoids, cysteine, uric acid, taurine, tyrosine, superoxide dismutase, lutein, zeaxanthin, cryptoxanthin, astaxanthin, lycopene, N-acetyl-cysteine, carnosine, gamma-glutamylcysteine, quercitin, lactoferrin, dihydrolipoic acid, citrate, Ginkgo Biloba extract, tea catechins, bilberry extract, vitamins E or esters of vitamin E, retinyl palmitate, and derivatives thereof.

Other therapeutic agents include squalamine, carbonic anhydrase inhibitors, alpha agonists, prostamides, prostaglandins, antiparasitics, antifungals, and derivatives thereof.

The amount of active agent or agents employed in the implant, individually or in combination, will vary widely depending on the effective dosage required and the desired rate of release from the implant. As indicated herein, the agent will be at least about 1, more usually at least about 10 weight percent of the implant, and usually not more than about 80, more usually not more than about 40 weight percent of the implant.

In addition to the therapeutic component, the intraocular implants disclosed herein may include effective amounts of buffering agents, preservatives and the like. Suitable water soluble buffering agents include, without limitation, alkali and alkaline earth carbonates, phosphates, bicarbonates, citrates, borates, acetates, succinates and the like, such as sodium phosphate, citrate, borate, acetate, bicarbonate, carbonate and the like. These agents advantageously present in amounts sufficient to maintain a pH of the system of between about 2 to about 9 and more preferably about 4 to about 8. As such the buffering agent may be as much as about 5% by weight of the total implant. Suitable water soluble preservatives include sodium bisulfite, sodium bisulfate, sodium thiosulfate, ascorbate, benzalkonium chloride, chlorobutanol, thimerosal, phenylmercuric acetate, phenylmercuric borate, phenylmercuric nitrate, parabens, methylparaben, polyvinyl alcohol, benzyl alcohol, phenylethanol and the like and mixtures thereof. These agents may be present in amounts of from 0.001 to about 5% by weight and preferably 0.01 to about 2% by weight.

In addition, the implants may include a solubility enhancing component provided in an amount effective to enhance the solubility of the retinoid component relative to substantially identical implants without the solubility enhancing component. For example, an implant may include a β-cyclodextrin, which is effective in enhancing the solubility of the retinoid component. The β-cyclodextrin may be provided in an amount from about 0.5% (w/w) to about 25% (w/w) of the implant. In certain implants, the β-cyclodextrin is provided in an amount from about 5% (w/w) to about 15% (w/w) of the implant.

In some situations mixtures of implants may be utilized employing the same or different pharmacological agents. In this way, a cocktail of release profiles, giving a biphasic or triphasic release with a single administration is achieved, where the pattern of release may be greatly varied.

Additionally, release modulators such as those described in U.S. Pat. No. 5,869,079 may be included in the implants. The amount of release modulator employed will be dependent on the desired release profile, the activity of the modulator, and on the release profile of the retinoid in the absence of modulator. Electrolytes such as sodium chloride and potassium chloride may also be included in the implant. Where the buffering agent or enhancer is hydrophilic, it may also act as a release accelerator. Hydrophilic additives act to increase the release rates through faster dissolution of the material surrounding the drug particles, which increases the surface area of the drug exposed, thereby increasing the rate of drug bioerosion. Similarly, a hydrophobic buffering agent or enhancer dissolve more slowly, slowing the exposure of drug particles, and thereby slowing the rate of drug bioerosion.

Various techniques may be employed to produce the implants described herein. Useful techniques include, but are not necessarily limited to, solvent evaporation methods, phase separation methods, interfacial methods, molding methods, injection molding methods, extrusion methods, co-extrusion methods, carver press method, die cutting methods, heat compression, combinations thereof and the like.

Specific methods are discussed in U.S. Pat. No. 4,997,652. Extrusion methods may be used to avoid the need for solvents in manufacturing. When using extrusion methods, the polymer and drug are chosen so as to be stable at the temperatures required for manufacturing, usually at least about 85 degrees Celsius. Extrusion methods use temperatures of about 25 degrees C. to about 150 degrees C., more preferably about 65 degrees C. to about 130 degrees C. An implant may be produced by bringing the temperature to about 60 degrees C. to about 150 degrees C. for drug/polymer mixing, such as about 130 degrees C., for a time period of about 0 to 1 hour, 0 to 30 minutes, or 5-15 minutes. For example, a time period may be about 10 minutes, preferably about 0 to 5 min. The implants are then extruded at a temperature of about 60 degrees C. to about 130 degrees C., such as about 75 degrees C. Preferably, the temperature is not substantially greater than the denaturation temperature associated with the therapeutic agent.

In addition, the implant may be coextruded so that a coating is formed over a core region during the manufacture of the implant.

Compression methods may be used to make the implants, and typically yield implants with faster release rates than extrusion methods. Compression methods may use pressures of about 50-150 psi, more preferably about 70-80 psi, even more preferably about 76 psi, and use temperatures of about 0 degrees C. to about 115 degrees C., more preferably about 25 degrees C.

In addition, the implants, particularly implants which are cut to the desired size and shape, such as wafer implants, may include an additive, such as a lubricant, that is effective to reduce the brittleness of the implant relative to substantially identical implants that do not have an additive. By providing such an additive in the implant, the amount of damaged or unusable implants due to breakage is substantially reduced.

The implants of the present invention may be inserted into the eye, for example the vitreous chamber of the eye, by a variety of methods, including placement by forceps or by trocar following making a 2-3 mm incision in the sclera. One example of a device that may be used to insert the implants into an eye is disclosed in U.S. Patent Publication No. 2004/0054374. The method of placement may influence the therapeutic component or drug release kinetics. For example, delivering the implant with a trocar may result in placement of the implant deeper within the vitreous than placement by forceps, which may result in the implant being closer to the edge of the vitreous. The location of the implant may influence the concentration gradients of therapeutic component or drug surrounding the element, and thus influence the release rates (e.g., an element placed closer to the edge of the vitreous may result in a slower release rate).

The present implants are configured to release an amount of the retinoid component effective to treat or reduce a symptom of an ocular condition, such as an ocular condition related to proliferative vitreal retinopathy, age related macular degeneration, diabetic retinopathy and retinitis pigementosa, among others.

The implants disclosed herein may also be configured to release the retinoid or additional therapeutic agents, as described above, which to prevent diseases or conditions, such as the following:

MACULOPATHIES/RETINAL DEGENERATION: Non-Exudative Age Related Macular Degeneration (ARMD), Exudative Age Related Macular Degeneration (ARMD), Choroidal Neovascularization, Diabetic Retinopathy, Acute Macular Neuroretinopathy, Central Serous Chorioretinopathy, Cystoid Macular Edema, Diabetic Macular Edema.

UVEITIS/RETINITIS/CHOROIDITIS: Acute Multifocal Placoid Pigment Epitheliopathy, Behcet's Disease, Birdshot Retinochoroidopathy, Infectious (Syphilis, Lyme, Tuberculosis, Toxoplasmosis), Intermediate Uveitis (Pars Planitis), Multifocal Choroiditis, Multiple Evanescent White Dot Syndrome (MEWDS), Ocular Sarcoidosis, Posterior Scleritis, Serpiginous Choroiditis, Subretinal Fibrosis and Uveitis Syndrome, Vogt-Koyanagi-Harada Syndrome.

VASCULAR DISEASES/EXUDATIVE DISEASES Retinal Arterial Occlusive Disease, Central Retinal Vein Occlusion, Disseminated Intravascular Coagulopathy, Branch Retinal Vein Occlusion, Hypertensive Fundus Changes, Ocular Ischemic Syndrome, Retinal Arterial Microaneurysms, Coat's Disease, Parafoveal Telangiectasis, Hemi-Retinal Vein Occlusion, Papillophlebitis, Central Retinal Artery Occlusion, Branch Retinal Artery Occlusion, Carotid Artery Disease (CAD), Frosted Branch Angiitis, Sickle Cell Retinopathy and other Hemoglobinopathies, Angioid Streaks, Familial Exudative Vitreoretinopathy, Eales Disease.

TRAUMATIC/SURGICAL: Sympathetic Ophthalmia, Uveitic Retinal Disease, Retinal Detachment, Trauma, Laser, PDT, Photocoagulation, Hypoperfusion During Surgery, Radiation Retinopathy, Bone Marrow Transplant Retinopathy.

PROLIFERATIVE DISORDERS: Proliferative Vitreal Retinopathy and Epiretinal Membranes, Proliferative Diabetic Retinopathy, Retinopathy of Prematurity (retrolental fibroplastic).

INFECTIOUS DISORDERS: Ocular Histoplasmosis, Ocular Toxocariasis, Presumed Ocular Histoplasmosis Syndrome (POHS), Endophthalmitis, Toxoplasmosis, Retinal Diseases Associated with HIV Infection, Choroidal Disease Associated with HIV Infection, Uveitic Disease Associated with HIV Infection, Viral Retinitis, Acute Retinal Necrosis, Progressive Outer Retinal Necrosis, Fungal Retinal Diseases, Ocular Syphilis, Ocular Tuberculosis, Diffuse Unilateral Subacute Neuroretinitis, Myiasis.

GENETIC DISORDERS: Retinitis Pigmentosa, Systemic Disorders with Accosiated Retinal Dystrophies, Congenital Stationary Night Blindness, Cone Dystrophies, Fundus Flavimaculatus, Best's Disease, Pattern Dystrophy of the Retinal Pigmented Epithelium, X-Linked Retinoschisis, Sorsby's Fundus Dystrophy, Benign Concentric Maculopathy, Bietti's Crystalline Dystrophy, pseudoxanthoma elasticum, Osler Weber syndrome.

RETINAL TEARS/HOLES: Retinal Detachment, Macular Hole, Giant Retinal Tear.

TUMORS: Retinal Disease Associated with Tumors, Solid Tumors, Tumor Metastasis, Benign Tumors, for example, hemangiomas, neurofibromas, trachomatis, and pyogenic granulomas, Congenital Hypertrophy of the RPE, Posterior Uveal Melanoma, Choroidal Hemangioma, Choroidal Osteoma, Choroidal Metastasis, Combined Hamartoma of the Retina and Retinal Pigmented Epithelium, Retinoblastoma, Vasoproliferative Tumors of the Ocular Fundus, Retinal Astrocytoma, Intraocular Lymphoid Tumors.

MISCELLANEOUS: Punctate Inner Choroidopathy, Acute Posterior Multifocal Placoid Pigment Epitheliopathy, Myopic Retinal Degeneration, Acute Retinal Pigment Epithelitis, Ocular inflammatory and immune disorders, ocular vascular malfunctions, Corneal Graft Rejection, Neovascular Glaucoma and the like.

In one embodiment, an implant, such as the implants disclosed herein, is administered to a posterior segment of an eye of a human or animal patient, and preferably, a living human or animal. In at least one embodiment, an implant is administered without accessing the subretinal space of the eye. For example, a method of treating a patient may include placing the implant directly into the posterior chamber of the eye. In other embodiments, a method of treating a patient may comprise administering an implant to the patient by at least one of intravitreal injection, subconjunctival injection, sub-tenon injections, retrobulbar injection, and suprachoroidal injection.

In at least one embodiment, a method of improving or maintaining vision in a patient comprises administering one or more implants containing one or more retinoid components, as disclosed herein to a patient by at least one of intravitreal injection, subconjunctival injection, sub-tenon injection, retrobulbar injection, and suprachoroidal injection. A syringe apparatus including an appropriately sized needle, for example, a 22 gauge needle, a 27 gauge needle or a 30 gauge needle, can be effectively used to inject the composition with the posterior segment of an eye of a human or animal. Repeat injections are often not necessary due to the extended release of the retinoid component from the implants.

In another aspect of the invention, kits for treating an ocular condition of the eye are provided, comprising: a) a container comprising an extended release implant comprising a therapeutic component including a retinoid component, such as a RAR agonist (e.g., tazarotene, tazarotenic acid, or mixtures thereof), and a drug release sustaining component; and b) instructions for use. Instructions may include steps of how to handle the implants, how to insert the implants into an ocular region, and what to expect from using the implants.

### Example 1

### Manufacture and Testing of Implants Containing an Retinoid Component and a Biodegradable Polymer Matrix

Biodegradable implants are made by combining a retinoid component, such as tazarotene, or tazarotenic acid, with a biodegradable polymer composition in a stainless steel mortar. Other retinoid components may include any one or more of the compounds described hereinabove. The combination is mixed via a Turbula shaker set at 96 RPM for 15 minutes. The powder blend is scraped off the wall of the mortar and then remixed for an additional 15 minutes. The mixed powder blend is heated to a semi-molten state at specified temperature for a total of 30 minutes, forming a polymer/drug melt.

Rods are manufactured by pelletizing the polymer/drug melt using a 9 gauge polytetrafluoroethylene (PTFE) tubing, loading the pellet into the barrel and extruding the material at the specified core extrusion temperature into filaments. The filaments are then cut into about 1 mg size implants or drug delivery systems. The rods have dimensions of about 2 mm long×0.72 mm diameter. The rod implants weigh between about 900 μg and 1100 μg.

Wafers are formed by flattening the polymer melt with a Carver press at a specified temperature and cutting the flattened material into wafers, each weighing about 1 mg. The wafers have a diameter of about 2.5 mm and a thickness of about 0.13 mm. The wafer implants weigh between about 900 μg and 1100 μg.

In-vitro release testing can be performed on each lot of implant (rod or wafer). Each implant may be placed into a 24 mL screw cap vial with 10 mL of Phosphate Buffered Saline solution at 37° C. and 1 mL aliquots are removed and replaced with equal volume of fresh medium on day 1, 4, 7, 14, 28, and every two weeks thereafter.

Drug assays may be performed by HPLC, which consists of a Waters 2690 Separation Module (or 2696), and a Waters 2996 Photodiode Array Detector. An Ultrasphere, C-18 (2), 5 μm; 4.6×150 mm column heated at 30° C. can be used for separation and the detector can be set at 264 nm. The mobile phase can be (10:90) MeOH-buffered mobile phase with a flow rate of 1 mL/min and a total run time of 12 min per sample. The buffered mobile phase may comprise (68:0.75:0.25:31) 13 mM 1-Heptane Sulfonic Acid, sodium salt-glacial acetic acid-triethylamine-Methanol. The release rates can be determined by calculating the amount of drug being released in a given volume of medium over time in μg/day.

The polymers chosen for the implants can be obtained from Boehringer Ingelheim or Purac America, for example. Examples of polymers include: RG502, RG752, R202H, R203 and R206, and Purac PDLG (50/50). RG502 is (50:50) poly(D,L-lactide-co-glycolide), RG752 is (75:25) poly(D,L-lactide-co-glycolide), R202H is 100% poly(D, L-lactide) with acid end group or terminal acid groups, R203 and R206 are both 100% poly(D, L-lactide). Purac PDLG (50/50) is (50:50) poly(D,L-lactide-co-glycolide). The inherent viscosity of RG502, RG752, R202H, R203, R206, and Purac PDLG are 0.2, 0.2, 0.2, 0.3, 1.0, and 0.2 dL/g, respectively. The average molecular weight of RG502, RG752, R202H, R203, R206, and Purac PDLG are, 11700, 11200, 6500, 14000, 63300, and 9700 daltons, respectively.

Microspheres can be manufactured with a tazarotene loading of 10% w/w in a biodegradable polymer composition of PLGA. The microspheres can then be sterilized by gamma irradiation at a dose of 2.5 to 4.0 mRad. One formula for a five-gram batch size is provided below:

| Component | Use | Quantity | |
|---|---|---|---|
| Phase I | | | |
| Polyvinyl Alcohol (PVA) | Stabilizer | 47.5 | grams |
| Purified Water | Solvent | 1600 | mL |
| Phase II | | | |
| Tazarotene | Active | Placebo or 1.0 | grams |
| Poly lactide-co-glycolide | Polymer/Vehicle | 4.50 | grams |
| 75:25 i.v. 0.43 or 0.65 | | | |
| Methylene Chloride | Solvent | 300 | mL |

In a five-liter beaker a solution of 3.0% PVA is manufactured using a high shear impeller and a stirring rate of 400 to 500 rpm at 80° C. Once in solution the stirring rate is reduced to 200 RPM to minimize foaming. PLGA is then dissolved in the methylene chloride. Once the PLGA is in solution, tazarotene is added and brought into solution.

Microspheres are then manufactured using a solvent evaporation technique. The PVA solution is vigorously stirred while slowly adding the tazarotene/PLGA solution. The emulsion is then allowed to stir over 48 hours to remove the methylene chloride. The microspheres are then rinsed, dried in vacuum and finally freeze dried. The microspheres are frozen at −50° C., then freeze dried for at least 12 hours at a 4 mbar minimum pressure (400 Pa).

### Example 2

### Controlled Release of Retinoic Acid Receptor Agonists as a Method of Preventing Proliferation of Retinal Pigmented Epithelium

An intraocular implant comprising an RAR agonist may be prepared as described in Example 1. The implant may include other actives or excipients as needed. The RAR agonist may be released from the implant by diffusion, erosion, dissolution or osmosis. Drug is released from the implant over a period of 7 days to over a period greater than one year. The polymeric implant may be comprised of bioerodible or non-erodible polymers. Bioerodible polymers may include a polyester, poly (ortho ester), poly(phosphazine), poly (phosphate ester), natural polymer such as gelatin or collagen, or a polymeric blend. The platform may be a solid implant, semisolid or viscoelastic. Administration of the drug delivery platform can be accomplished via intravitreal, subconjunctival, subretinal, retro-bulbar implantation or injection. This invention describes the controlled or sustained delivery of retinoic acid receptor (RAR) agonists for the treatment of retinal diseases associated with the proliferation of the retinal pigment epithelium.

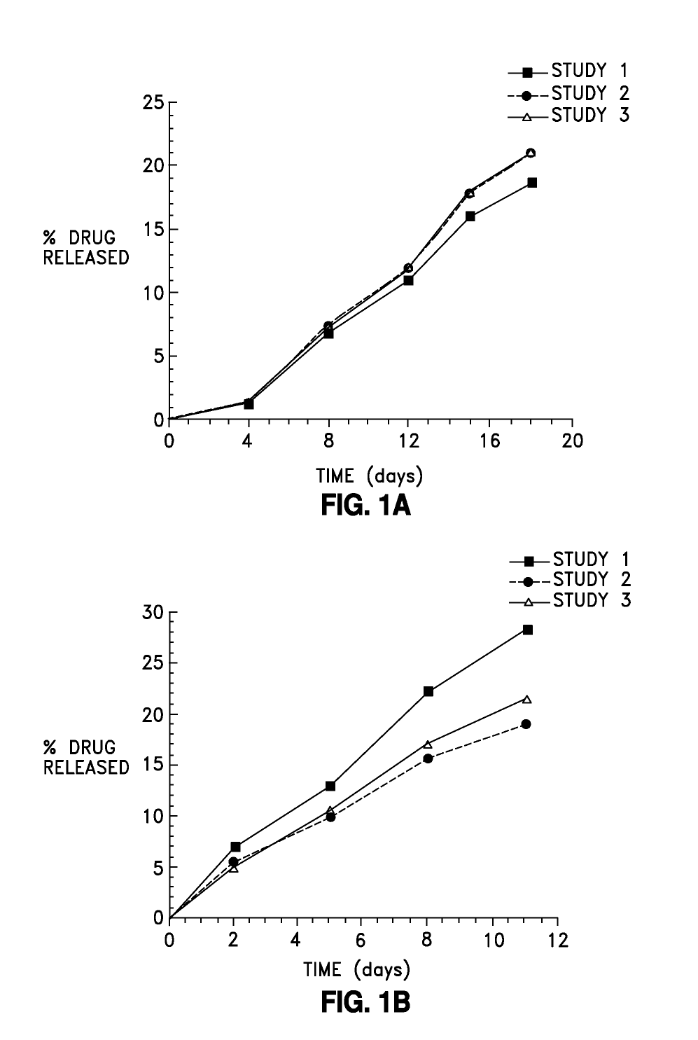

FIG. 1A and FIG. 1B illustrate the cumulative percent in-vitro release of tazarotene from PLA and PLGA microsphere implants.

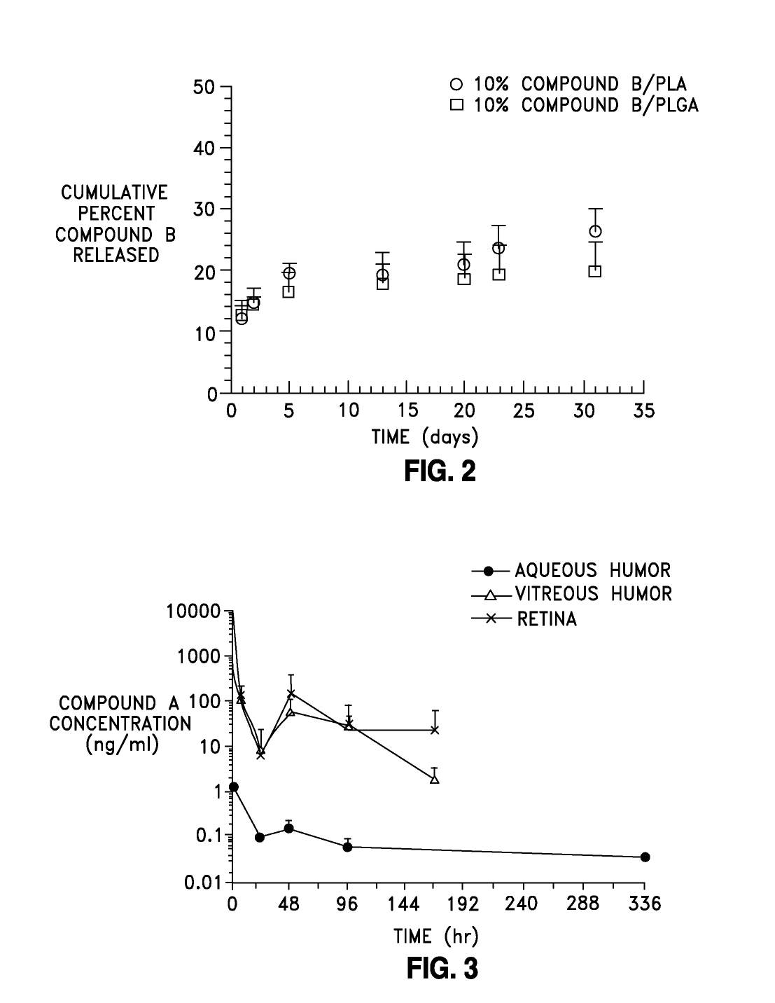

FIG. 2 illustrates the cumulative percent in vitro release of tazarotenic acid from polylactide (PLA) and poly(lactide-co-glycolide) (PLGA) implants.

Thus, direct or local administration of the present implants into an ocular region circumvents the side effects and toxicity of systemic administration while reducing the need for multiple intravitreal bolus injections. Controlled or sustained delivery of the retinoid from the implant provides for one time dosing of the retinoid at the time of surgery.

### Example 3

### Method of Sterilization of PLGA and PLA Microspheres and Bioerodible Implants

Tazarotene-containing microspheres (75:25 PLGA) were prepared as described in Example 1. PLA and PLGA microspheres or implants were gamma irradiated at a dose ranging from 1.0 to 4.5 mRad, such as 2.5 to 4.0 mRad, at low temperatures, such as at 0° C. The temperature was lowered by the addition of cold packs to the sterilization carton or by lowering the temperature of the environment. Gamma irradiation results in significant aggregation of the microspheres.

There is a significant aggregation observed as a result of gamma irradiation in both drug loaded and placebo microspheres. When the microspheres were sterilized at a reduced temperature the aggregation was prevented. Microspheres sterilized at ambient temperatures show a marked aggregation. This includes low molecular weight and high molecular weight PLGA implants loaded with 10% tazarotene. A strong shift towards increase in volume average particle size is observed for all sublots. Those sublots sterilized at reduced temperatures show an almost superimposeable volume and number average particle size distribution with their non-sterilized counterparts.

Thus, PLA and PLGA microspheres have been terminally sterilized thereby reducing the need for aseptic processing, heat or steam sterilization or the use of ethylene oxide. The stability of the PLA and PLGA microspheres and implants sterilized by this method is improved relative to other methods. This is important as the PLA and PLGA polymers are heat and moisture labile. Additionally, the rate of drug release from monolithic implants is the integrated result of diffusional and degradation processes. As such surface area changes encountered with aggregation will cause significant variability in drug release and implant degradation profile.

### Example 4

### Sterile Bioerodible Retinal Plug for Intraocular Drug Delivery

The bioerodible plug comprises of one or more bioerodible polymers, a retinoid and other active compounds or excipients. The retinoid is present as the active for the treatment of proliferative vitreal retinopathy (PVR) and retinal neovascularization or to improve the biocompatibility of the device. The bioerodible polymers may include a polyester, poly (ortho ester), poly(phosphazine), poly (phosphate ester), natural polymer such as gelatin or collagen, or a polymeric blend.

The components of the device are extruded as a homogeneous device in the shape of a plug: The plug is then inserted into the vitreous cavity through the sclera, choroid and retina. The distal end of the plug protrudes into the vitreous cavity. A hole drilled into the proximal end of the device is used to suture the plug to the sclera. The plug may be loaded with 0.1 to 40% w/w of pharmacologically active compounds. Drug is released from the plug into the sclera, choroid, retina and vitreous cavity over a period of 7 days to over a period greater than one year.

Incorporation of retinoids (tazarotenic acid, tazarotene, or other retinoid receptor agonist) into the plug can improve the biocompatibility of the implant and provide a therapeutic effect in the prevention or treatment of PVR. The plug may be optimized to resist scleral and choroidal erosion of the plug. This would prevent disinsertion or fragmentation into the vitreous cavity. This may be accomplished by altering the surface finish of the plug, coating the plug with another biodegradable semipermeable polymer or the addition of another polymer to the blend. Advantageously, organic solvents are not needed for the incorporation of the active agent and excipients into the polymeric matrix. The active compounds, polymers and excipients are milled prior to extrusion. The plug is a homogeneous system that provides for ease of manufacturing and scale-up by simple extrusion technologies. Further, the polymeric plug may also be manufactured by injection molding. The mechanism and rate of drug release may be controlled by choice of polymer, polymer molecular weight, polymer crystallinity, copolymer ratios, processing conditions, surface finish, geometry, excipient addition and polymeric coatings. Drug may be released from the device by diffusion, erosion, dissolution or osmosis.

The release of the retinoid from the plug may include an initial burst of 10% of the retinoid, and an additional 10% over the first month after placement in an eye.

Some related information may be found in U.S. Pat. Nos. 4,712,500; 5,466,233, and in Kimura, Hideya et. al. A New Vitreal Drug Delivery System using an Implantable Biodegradable Polymeric Device, Invest Ophthalmol Vis Sci. 1994; 35 : 2815-2819; and Hashizoe, Mototane et. al. Scleral Plug of Biodegradable Polymers for Controlled Drug Release in the Vitreous, Arch Ophthalmol. 1994; 112: 1380-1384.

### Example 5

### Subconjunctival and Periocular Vitreous Drug Delivery of Prodrugs with Improved Safety

This example teaches that subconjunctival and periocular administration of ester prodrugs provides a higher therapeutic index than direct intraocular administration of the prodrugs.

The vitreous has a limited capacity to hydrolyze ester prodrugs into their active parent. Although it is counterintuitive, subconjunctival or periocular administration of ester prodrugs is more efficient than direct intraocular administration. This is contrary to what would be anticipated from the current literature. The blood-retinal barriers provide a significant constraint to vitreoretinal drug delivery, as discussed herein. Circumventing these barriers by direct intraocular administration is the current practice and thought to be the most efficient mode of delivery. Few compounds are delivered by subconjunctival or periocular administration as this is much less efficient than direct intraocular administration.

In this example it is demonstrated that ester prodrugs can actually be delivered to the vitreous by subconjunctival administration more efficiently than by direct intraocular administration. This is believed to be a result of the differential esterase activity in the choroid and iris-ciliary body versus the vitreous. Interestingly, the ubiquitous nature of esterases in the iris-ciliary body and the choroidal circulation allow for a more efficient vitreous delivery of drug from periocular administration than intraocular injection.

This example demonstrates improvements in pharmacotherapy of compounds with low therapeutic indexes directed at the posterior ocular structures. Thus, this example demonstrates the (i) use of subconjunctival or periocular administration of ester prodrugs for vitreal drug delivery; (ii) use of subconjunctival or periocular administration of ester prodrugs for retinal drug delivery; (iii) subconjunctival or periocular delivery of retinoid ester prodrugs; (iv) subconjunctival or periocular delivery of ester prodrugs of carboxylic acids; (v) subconjunctival or periocular delivery of ester prodrugs of alcohols; (vi) use of subconjunctival or periocular administration of ester prodrugs for the delivery of compounds to the posterior structures of the eye including: uveal tract, vitreous, retina, choroid and retinal pigmented epithelium; (vii) use of subconjunctival or periocular administration of non-ester prodrugs where the enzymes responsible for bioreversion are at a higher activity in the subconjunctival or periocular space than the vitreous. These are for the delivery of compounds to the posterior structures of the eye including: uveal tract, vitreous, retina, choroid and retinal pigmented epithelium.

Thus, implants and methods are provided that will allow for a more efficient vitreoretinal delivery of drugs and thereby allow for improvements in their therapeutic index.

This example utilizes the unique esterase distribution within the eye to deliver drugs to the back of the eye in a more efficient manner. The subconjunctival or periocular space can serve as a depot for an ester prodrug. Facile hydrolysis upon intraocular penetration is more efficient than intravitreal hydrolysis due to the ubiquitous nature of esterases in the iris-ciliary process and the choroidal circulation. The result is: (i) the ability to utilize lipophilic prodrugs that can enhance trans-retinal penetration while; (ii) lowering intraocular concentrations of the parent prodrug relative to the parent prodrug concentrations achieved after intraocular injection. This is due to more efficient hydrolysis to the active drug; (iii) an improved intraocular prodrug/drug ratio; and (iv) the creation of a lipophilic depot of compound in the subconjunctival or periocular space for sustained delivery.

Ethyl 6-[(4,4-dimethylthiochroman-6-yl)ethynyl]nicotinate (tazarotene) is the ethyl ester of the active retinoid 4,4-dimethyl-6-[2′-(5″-carboxy-2″-pyridyl)-ethynyl]-thiochroman (tazarotenic acid).

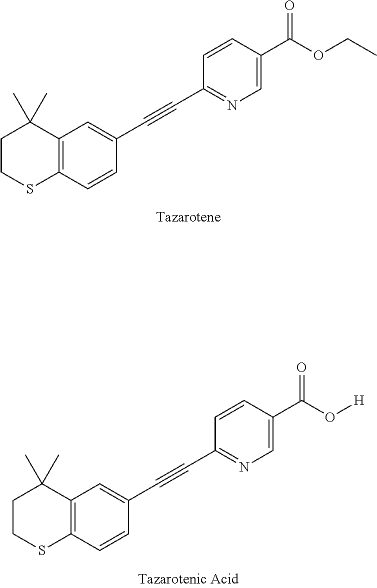

Tazarotene is a lipophilic prodrug of tazarotenic acid with a logP of 4.3 and a solubility of 1 μg/mL in water. Retinoids are known to be therapeutic in treating several conditions of the retina and retinal pigmented epithelium such as retinitis pigmentosa and proliferative vitreal retinopathy. Unfortunately, retinoids are also known to cause cataracts. This is most likely due to the effect of the retinoid on the lens epithelium. Highly lipophilic retinoids have the additional disadvantage of favorable partitioning into the lipophilic lens epithelium. Minimizing the amount of tazarotene in the vitreous relative to tazarotenic acid may improve the compounds therapeutic index.

General disposition of intraocular and subconjunctivally administered tazarotene and tazarotenic acid was assessed. Briefly, albino rabbits were dosed via intraocular injection with 1.25 μg of tazarotene or tazarotenic acid. Injection was made mid-vitreous. After dosing the vitreous, retina and aqueous humor concentrations of tazarotene and tazarotenic acid were determined at 0.5, 1, 2, 4, 8, 12 and 24 hours post dosing. The data demonstrate that tazarotenic acid is generated from tazarotene in the vitreous. Additionally, the tazarotenic acid concentrations asymptotically approach approximately 10 ng/mL. It appears that the vitreous esterase activity is overwhelmed with the maximal vitreous concentration of tazarotenic acid obtainable after direct intraocular implantation equal to 10 ng/mL. tazarotenic acid is eliminated in an apparent first order process from the vitreous with a half-life of 4.24 hours after midvitreous dosing of 1.25 μg of tazarotenic acid.

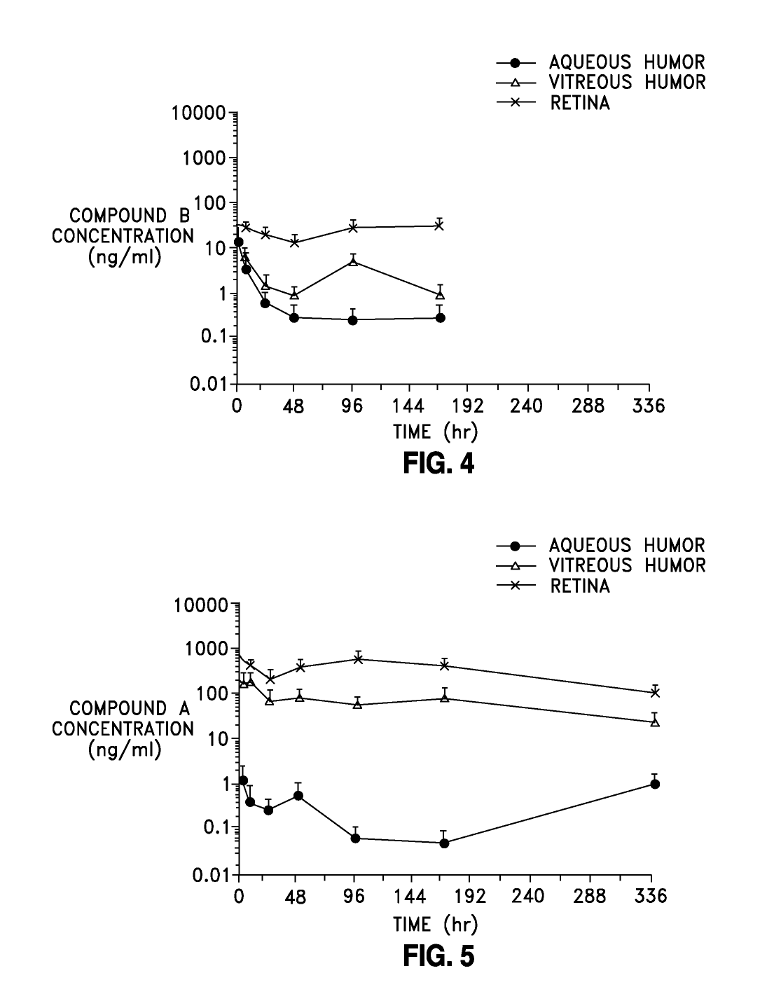

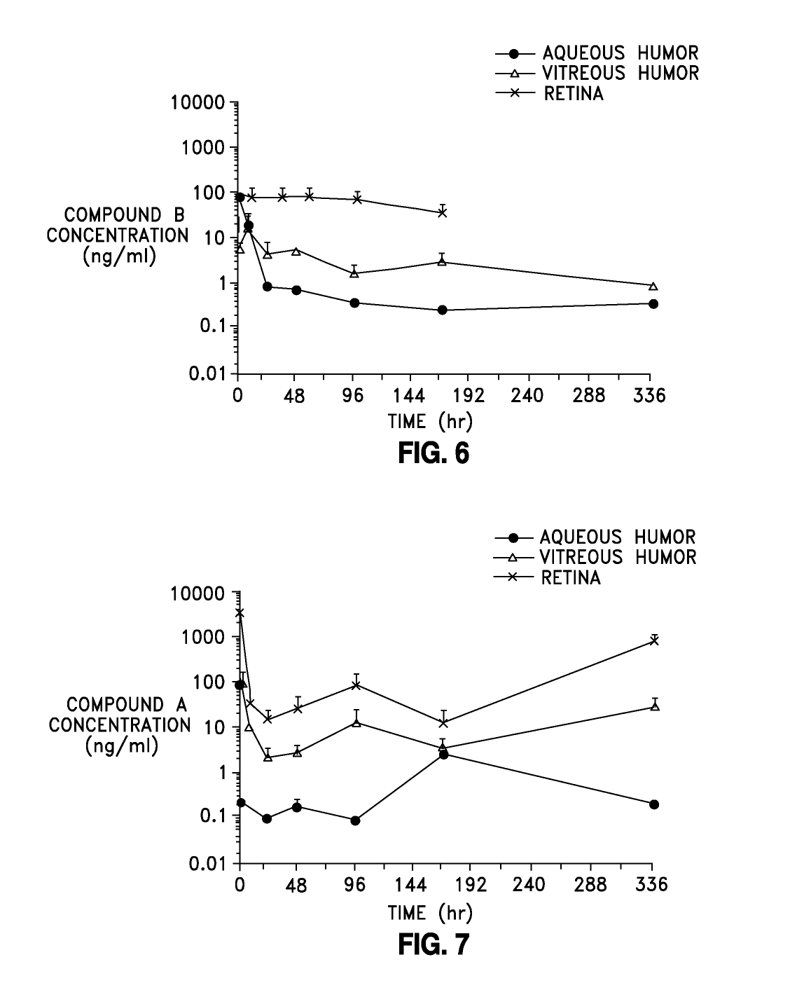

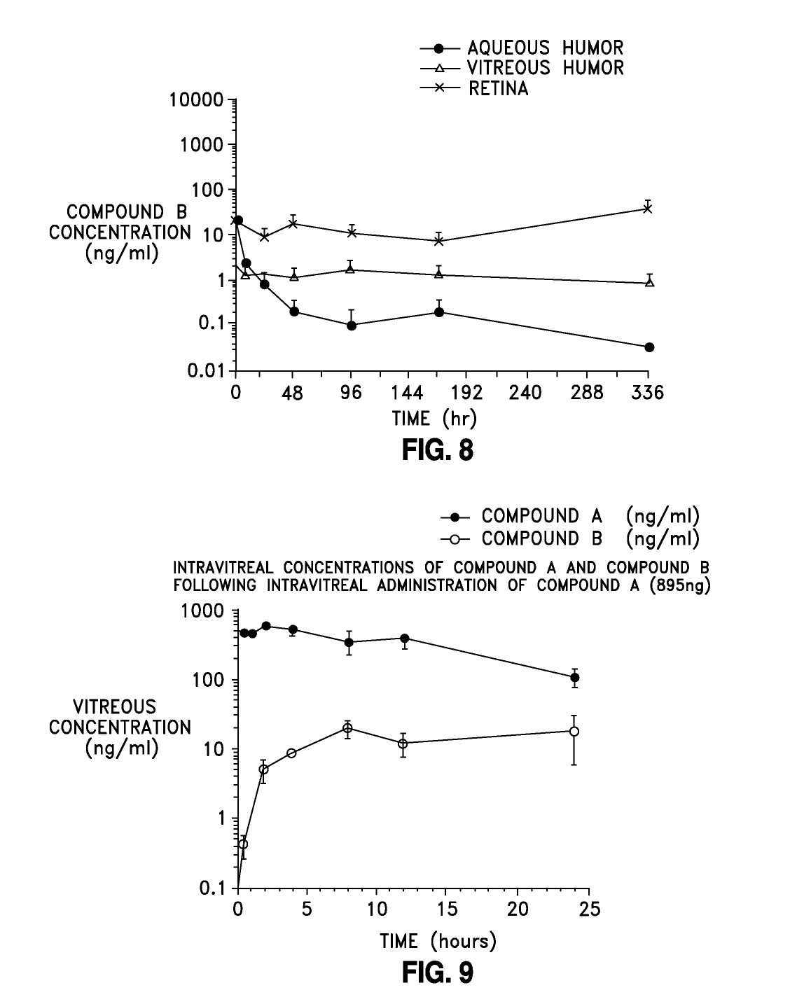

Tazarotene was also dosed in the subconjunctival space. Three dosage forms were evaluated: A tazarotene aqueous suspension (1 mg), a tazarotene subconjunctival olive oil solution (1 mg), and an tazarotene poly (lactide-co-glycolide) microsphere suspension. After dosing, the vitreous, retina and aqueous humor concentrations of tazarotene and tazarotenic acid were determined at 2, 8, 24, 48, 96, 168 and 336 hours post dosing. It was observed that subconjunctival administration achieved significant levels of tazarotene and tazarotenic acid in the ocular tissues. More importantly, the ratio of tazarotene to tazarotenic acid was significantly lower, indicating a higher capacity to hydrolyze the ester prodrug into its parent by this route. The vitreous concentration data is summarized in Table 1. The vitreous concentration time profiles are summarized in FIGS. 3 through 9.

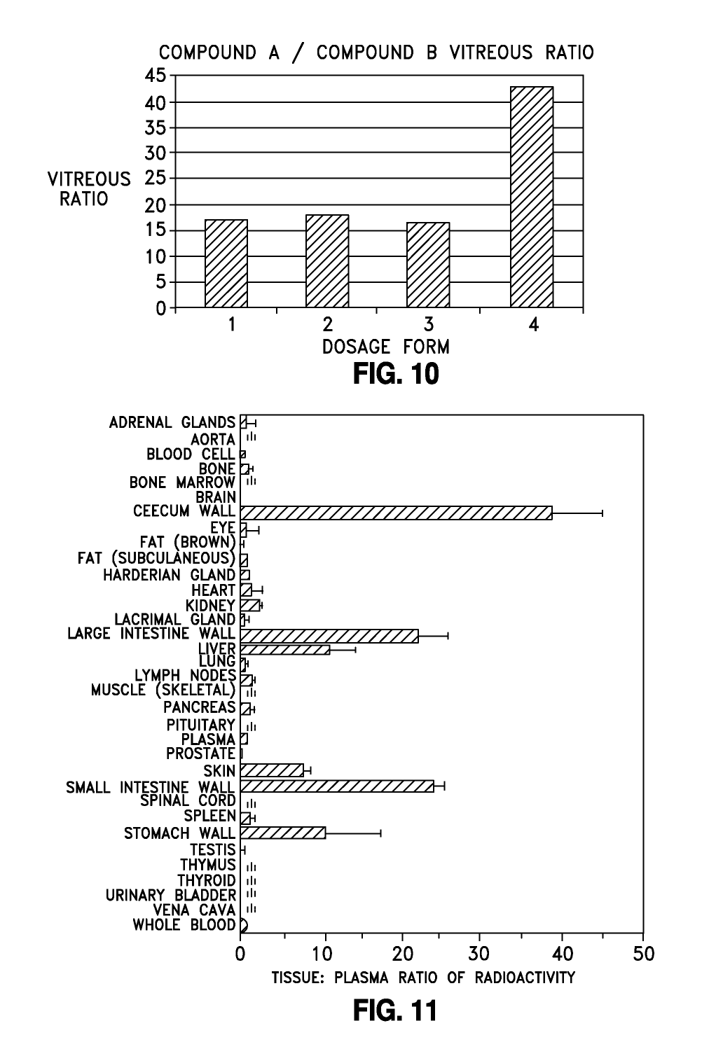

The data shows a more efficient delivery of tazarotenic acid from subconjunctival delivery compared with intravitreal delivery. The tazarotene/tazarotenic acid ratio is significantly lower from subconjunctival deliver as shown in FIG. 10. This surprising result is believed to be due to the higher esterase activity of the choroid and iris-ciliary body when compared to the retina. It is also important to note that concentrations of the retinoids tazarotene and tazarotenic acid were maintained at low effective levels for a period of 336 hours.

Thus, the present example describes the (i) use of subconjunctival or periocular administration of ester prodrugs for vitreal drug delivery; (ii) use of subconjunctival or periocular administration of ester prodrugs for retinal drug delivery; (iii) subconjunctival or periocular delivery of retinoid ester prodrugs; (iv) subconjunctival or periocular delivery of ester prodrugs of carboxylic acids; (v) subconjunctival or periocular delivery of ester prodrugs of alcohols; (vi) use of subconjunctival or periocular administration of ester prodrugs for the delivery of compounds to the posterior structures of the eye including: Uveal tract, vitreous, retina, choroid and retinal pigmented epithelium; (vii) use of subconjunctival or periocular administration of non-ester prodrugs where the enzymes responsible for bioreversion are at a higher activity in the subconjunctival or periocular space than the vitreous. These are for the delivery of compounds to the posterior structures of the eye including: Uveal tract, vitreous, retina, choroid and retinal pigmented epithelium.

TABLE 1
Vitreous Concentrations of tazarotene and tazarotenic
acid after Intravitreal and Subconjunctival Dosing.

| | Mean Vitreous | Mean Vitreous | tazarotene/ |
|---|---|---|---|
| | Concentration | Concentration | tazarotenic |
| Dosage Form | tazarotene | tazarotenic acid | acid ratio |
| Intravitreal | 417.0 | 9.9 | 42.0 |
| Injection (1.25 μg) | | | |
| Subconjunctival | 42.0 | 2.5 | 16.8 |
| Suspension (1 mg) | | | |
| Subconjunctival | 21.9 | 1.4 | 16.1 |
| Microspheres (1 mg) | | | |
| Subconjunctival Oil | 96.2 | 5.43 | 17.7 |
| Solution (1 mg) | | | |

### Example 6

### Hydrophilic Retinoids with Reduced Ocular Side Effects

This example describes hydrophilic retinoids with good oral and topical bioavailability with improved ocular side effect profiles. This example describes the use of retinoids with log partition coefficients (log P) less than 3.0 that have reduced ocular side effects.

Tazarotenic Acid Ocular Concentrations

TABLE 2
Tazarotene and Tazarotenic Acid plasma concentrations
after topical and systemic delivery.

| | | | TAZAROTENIC | |
|---|---|---|---|---|
| | | TAZAROTENE | ACID | |
| Route | Dose (Conc.) | Cmax (ng/mL) | Cmax (ng/mL) | Study |
| Topical Gel | 0.1% 2% BSA | Tazarotene | 0.241 | R-168-155-8757 |
| | 0.1% 7% BSA | concentrations | 0.83 | R190168-022 |
| | 0.1% 15% BSA | BLQ for >90% | 4.80 | R168-152-8606 |
| | 0.1% 20% BSA | | 12.0 | R-168-153-8606 |
| | 0.1% Phase 3 | | 90% <1 ng/mL | |
| Oral | 1.1 mg/Day | 95% <0.1 ng/mL | 28.9 | 190168-018P-00 |
| Oral | 3.0 mg/Day | <0.1 ng/mL | 81.6 | 190168-019P |
| Oral | 6.0 mg/Day | <0.1 ng/mL | Cₘₐₓ 227 | 190168-044P |
| | | | C_{trough} 2.56 | |

Table 2. summarizes the vitreous retinoid levels after topical and oral dosing of tazarotene. For the most part tazarotene is not observed in the plasma after topical or oral administration. Facile hydrolysis by pre-systemic metabolism rapidly generates the free acid. Tazarotenic acid plasma concentrations (Cmax, maximal plasma level) from topical administration range from 0.25 ng/mL to 12 ng/mL. It is important to note that these are plasma concentrations and the eye plasma distribution ratio is 0.02. Over 90% of all patients in the phase 3 clinical trials had concentrations of the parent compound, tazarotene, <1 ng/mL with the highest being 6 ng/mL.

Oral delivery of 1.1 mg and 6 mg multiple doses led to 28.9 ng/mL and 227 ng/ml peak levels with a 2.56 ng/mL trough. This corresponds to a maximum possible 4 ng/mL ocular level for the highest dose. FIG. 11 depicts the tissue distribution of tazarotenic acid. The eyes show a 2% tissue/plasma ration in the rat. It should be noted that tazarotenic acid is 99% protein bound and distribution is limited to unbound drug. Hence, the overwhelming majority of ocular tazarotenic acid concentrations are most probably in the anterior tissues. Further, the log P of tazarotenic acid is calculated to be 2.53 hence it is not expected to display efficient penetration of the blood-retinal barriers.

TABLE 3

| Compound | Bexarotene | Acetretin |
|---|---|---|
| Chemical Structure | 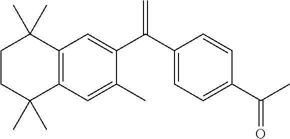 | 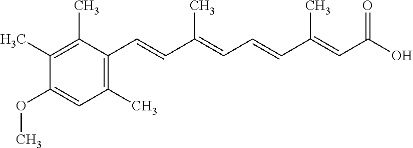 |
| log P¹ | 8.75 | 5.73 |
| log D¹ | 5.93 | 3.14 |
| pH 7.4 | | |
| Cataract | Yes | Yes |
| Night | Yes | Yes |
| Blindness | | |
| Pseudotumor | Yes | Yes |
| Cerebri | | |
| Depression | Yes | Yes |
| Nervousness/ | Yes | Yes |
| Agitation | | |
| | Isotretinoin | Tazarotene |
| Chemical Structure | 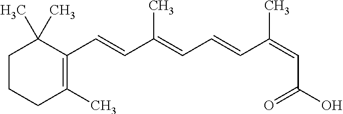 | 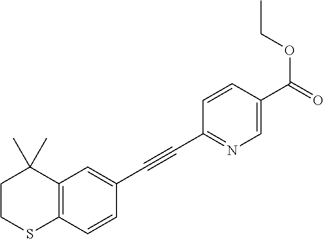 |
| log P¹ | 6.83 | |
| log D¹ | 4.25 | 6.21 (4.30 measured) |
| pH 7.4 | | |
| Cataract | Yes | N/A² |
| Night | Yes | N/A² |
| Blindness | | |
| Pseudotumor | Yes | N/A² |
| Cerebri | | |
| Depression | Yes | N/A² |
| Nervousness/ | Yes | N/A² |
| Agitation | | |

| Compound | Tazarotenic Acid |
|---|---|
| Chemical Structure | 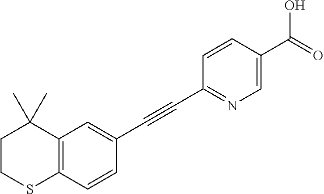 |
| log D¹ | 2.52 |
| pH 7.4 | |
| Cataract | No |
| Night | No |
| Blindness | |
| Pseudotumor | No |
| Cerebri | |
| Depression | No |
| Nervousness/ | No |
| Agitation | |

¹Calculated values, ACD/PhysChem computer software (v5.0)
²Converted to tazarotenic acid upon absorption.

The foregoing compounds of the present example may be provided in any of the intraocular implants described herein.

### Example 7

### Targeted Retinal Drug Delivery by Subconjunctival and Periocular Administration of Sustained Delivery Systems

This example describes the subconjunctival and periocular administration of compounds providing for higher retinal concentrations than obtained from immediate release or direct intraocular administration. It has been observed that compounds penetrating the RPE develop higher retinal/vitreous concentration ratios than when delivered by intraocular administration. More significant is the fact that controlled or sustained delivery from the subconjunctival route results in significantly higher retinal/vitreous ratios than non-controlled delivery.

This example demonstrates improvements in pharmacotherapy of drugs directed at the posterior ocular structures. This example describes (i) use of sustained or controlled, subconjunctival or periocular, administration of drugs to target choroid, RPE and retinal drug delivery; (ii) use of PLGA microspheres to sustain or control, subconjunctival or periocular, administration of drugs resulting in higher retina/vitreous concentration ratios; (iii) use of monolithic PLGA implants to sustain or control, subconjunctival or periocular, administration of drugs resulting in higher retina/vitreous concentration ratios; (iv) use of bioerodible controlled delivery systems to sustain or control, subconjunctival or periocular, administration of drugs resulting in higher retina/vitreous concentration ratios; (v) use of non-bioerodible controlled delivery systems to sustain or control, subconjunctival or periocular, administration of drugs resulting in higher retina/vitreous concentration ratios; (vi) currently, the only methods for achieving therapeutic retinal concentrations of drugs includes high dose systemic administration or direct intraocular implantation or injection. All of these approaches carry significant risk especially for drugs that may possess some intraocular toxicity. This examples provides more efficient choroid, RPE and retinal delivery of drugs and thereby allows for improvements in their therapeutic index.

As discussed herein, delivery of drugs to the retina, vitreous and uveal tract is typically achieved by high systemic dosing or direct intra-ocular injections. This example shows that the retinal/vitreal drug concentration ratio for compounds delivered from intraocular injection or non-sustained release subconjunctival administration is relatively low. In contrast, delivery of compounds to the retina from sustained subconjunctival administration results in a dramatic increase in the retinal/vitreous concentration ratio of drugs (See Table 4).

Compounds are eliminated from the vitreous by diffusion to the retro-zonular space with clearance via the aqueous humor or by trans-retinal elimination. Most compounds utilize the former pathway while lipophilic compounds and those with trans-retinal transport mechanisms will utilize the latter. In both cases the result is a relatively low retinal/vitreous concentration ratio of drug. Penetration of drug from periocular administration can proceed by trans-scleral diffusion with penetration through the RPE or diffusion to the iris root followed by posterior diffusion of drug into the vitreous. With pulsatile dosing the result is a relatively low retinal/vitreal concentration gradient. In this example we show that by utilizing sustained or controlled drug delivery systems we can achieve higher retinal/vitreal drug concentration ratios. Sustained delivery allows the eye to develop and maintain steady-state rate processes. The result includes (i) a targeting of the drug to the retina over the vitreous; (ii) a lower vitreous concentration for a given retinal level relative to other routes of delivery; (iii) improved efficacy for compounds acting at the choroid, RPE or retinal; and (iv) potential reduction in intraocular side effects due to reduced vitreous levels.

Briefly, albino rabbits were dosed via intraocular injection with 1.25 μg of tazarotene or tazarotenic acid. Injection was made mid-vitreous. After dosing the vitreous, retina and aqueous humor concentrations of tazarotene and tazarotenic acid were determined at 0.5, 1, 2, 4, 8, 12 and 24 hours post dosing.

Tazarotene was also dosed in the subconjunctival space. Three dosage forms were evaluated: A tazarotene aqueous suspension (1 mg), a tazarotene subconjunctival olive oil solution (1 mg), and a tazarotene poly (lactide-co-glycolide) microsphere suspension. After dosing, the vitreous, retina and aqueous humor concentrations of tazarotene and tazarotenic acid were determined at 2, 8, 24, 48, 96, 168 and 336 hours post dosing.

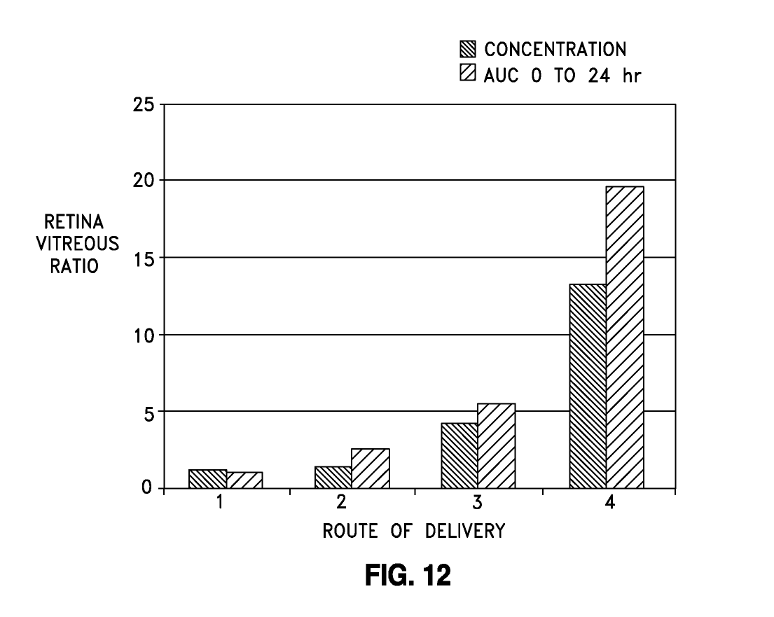

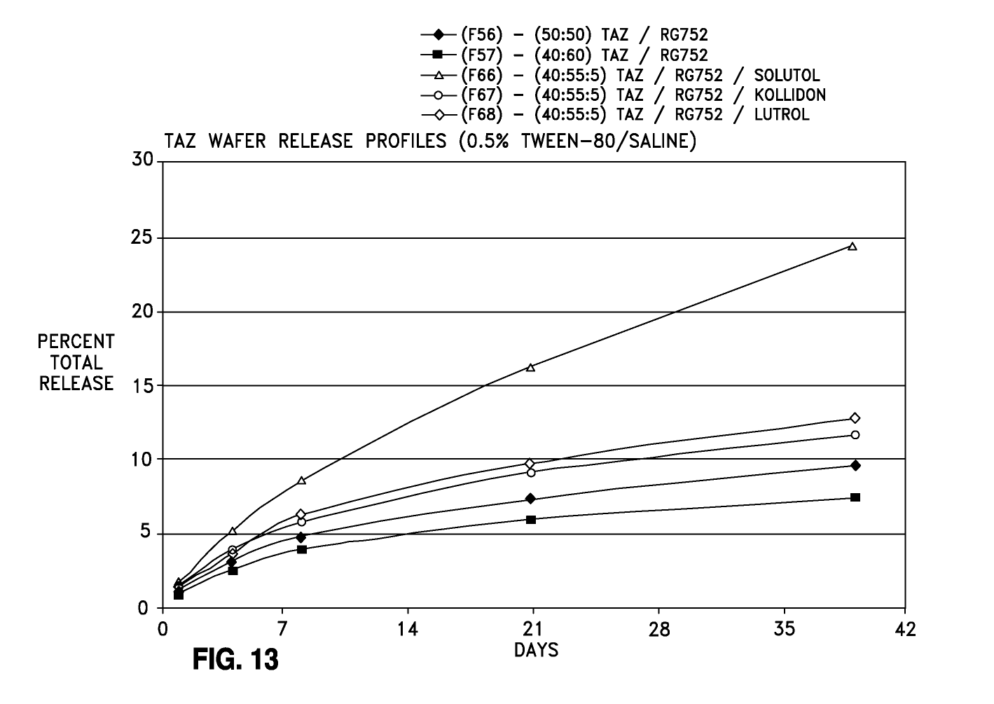

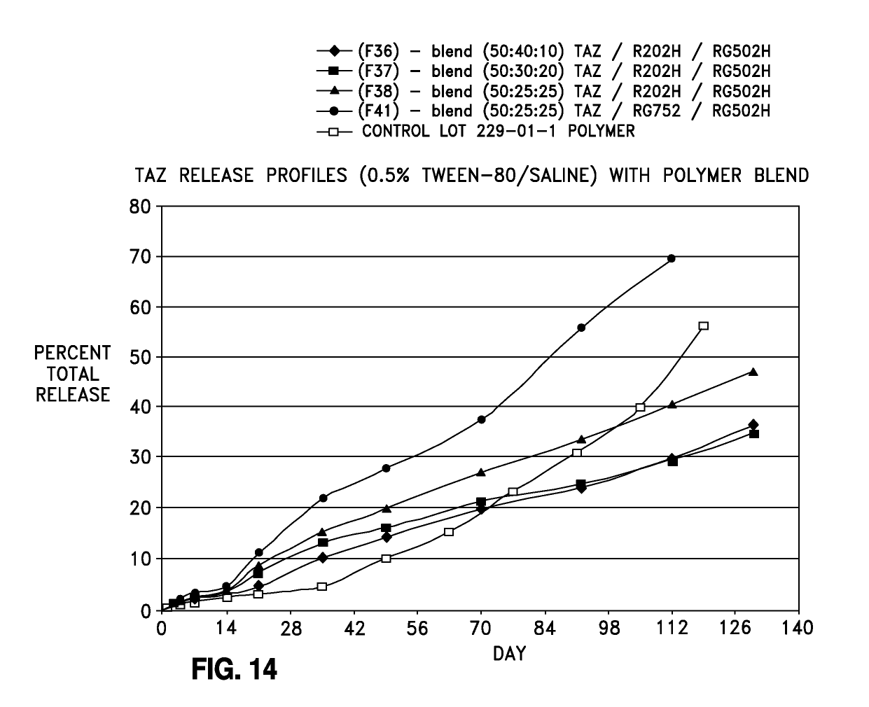

The mean retinal/vitreal concentration ratio as well as the retinal/vitreal AUC₀₋₂₄ₜₗₐₛₜ ratio are given in Table 4. The data show a higher retinal/vitreous ratio for tazarotene delivered from sustained release. This is graphically depicted in FIG. 12. A higher ratio of tazarotene in the retina is achieved with sustained release when compared with both non-sustained subconjunctival or direct intraocular administration. Thus, this examples provides implants and methods for (i) targeting of the drug to the retina over the vitreous; (ii) a lower vitreous concentration for a given retinal level relative to other route of delivery; (iii) improved efficacy for compounds acting at the choroid, RPE or retinal level; or (iv) potential reduction in intraocular side effects due to reduced vitreous levels relative to the retina.

TABLE 4
Vitreous Concentrations of TAZAROTENE and
TAZAROTENIC ACID after Intravitreal and
Subconjunctival Dosing.

| | Intra- | Subconj. | Subconj. | |
|---|---|---|---|---|
| | vitreal | Sus- | Oil | Subconj. |
| | Injection | pension | Solution | Microspheres |
| Dosage Form | (1.25 μg) | (1 mg) | (1 mg) | (1 mg) |
| Mean Retinal | 493.4 | 57.1 | 387.4 | 287.0 |
| Concentration | | | | |
| TAZAROTENE | | | | |
| (ng/mL) | | | | |
| Mean Vitreous | 417.0 | 42.0 | 96.2 | 21.9 |
| Concentration | | | | |
| TAZAROTENE | | | | |
| (ng/mL) | | | | |
| Retinal/Vitreous | 1.18 | 1.36 | 4.18 | 13.1 |
| Concentration | | | | |
| Ratio | | | | |
| Mean Retinal AUC | 8465 | 17000 | 127000 | 90100 |
| TAZAROTENE | | | | |
| (ng * hr/mL) | | | | |
| Mean Vitreous | 8611 | 6880 | 23400 | 4650 |
| AUC | | | | |
| TAZAROTENE | | | | |
| (ng * hr/mL) | | | | |
| Retinal/Vitreous | 0.98 | 2.47 | 5.43 | 19.38 |
| AUC Ratio | | | | |

### Example 8

### Tazarotene Wafer Implants with Additives

Tazarotene wafers were prepared by mixing tazarotene, RG 752 & the additive (Solutol®, Kollidon®, or Lutrol®). The additive may be a polyvinyl acetate. The powder mixture was melted and poured into a polymer melt. This was then compressed to the desired thickness & cut with a 2.5 mm trephine. These wafers are biodegradable drug delivery systems with improved mechanical properties and tailored release rates especially well suited for subconjunctival ocular drug delivery, as shown in FIG. 13.

Wafers prepared without the additives may be too brittle or fragile to cut, isolate, depending on the drug load and the polymer used. The additives may act as lubricants that make the wafers less brittle under the same processing conditions and thus reduces loss due to breakage and increase the yield. The choice and amount of additive used can tailor to the drug release rate, either accelerate or decelerate the release rate.

### Example 9

### Tazarotene Drug Delivery System with Blended Polymers

Tazarotene implants were prepared by mixing tazarotene and polymers RG502H with R202H or RG502H with RG752. The powder mixture was melted, pelletized, and extruded at specified temperatures depending on the drug load and polymer mix ratio. The implants provide a more predictable and linear release profile that can be achieved by adjusting the polymer blend ratio, as shown in FIG. 14. One of the benefits is to alleviate or reduce the typical sigmoidal release curve obtained with only one polymer.

### Example 10

### Tazarotene Containing Intraocular Implants

The intended delivery was for 3-6 months with an initial drug load of 0.1 mg-0.5 mg per implant. Four different polymers, RG502, RG502H, RG752, and R202H were used to formulate Tazarotene. Formulation #9, 50% Tazarotene (500 μg dose) in RG752 was selected by Allergan for their GLP PK/Tox studies. Implants were also made which contained 50 μg of tazarotene. Also discussed herein are formulations having a more linear release using polymer blends and wafer formulations with additives to improve their mechanical properties.

The initial Tazarotene PLGA (PLA) intra-ocular implant was intended for a 3-6 month delivery with a drug load of 0.1 to 0.5 mg. Two methods of preparing tazarotene implants may be used, polymer melt and powder compaction. The former method involved first mixing the polymer with Active Pharmaceutical Ingredient (API), then melting the resulting powder blend at a temperature lower than the melting point of API to prevent its decomposition, and finally extruding the API polymer blend into filaments. The latter method was performed by first mixing the polymer with API, then compacting the powder blend into the extrusion barrel, and finally heating the barrel and extruding the filaments. The polymer melt method may be preferred over the powder compaction method since the latter can create a dust cloud during compaction process. Extruded filaments were then cut to the appropriate weight of 1 mg+/−10% as rod-shaped implants or drug delivery systems (DDSs).

Due to the low melting point of Tazarotene (m.p.=103-106° C.), only polymers with molten range below Tazarotene's melting point could be effectively used in the process. Of the polymers available to us, only the ones with an inherent viscosity (I.V.) of 0.2 dl/g or lower were selected. Polymers with higher inherent viscosities become molten at temperatures higher than the melting point of Tazarotene and could cause degradation of tazarotene.

A second geometry used in making the Tazarotene DDS was the wafer. It was believed that this configuration could be more easily implanted subconjunctivally than the rod. The wafer process was performed by taking the melted polymer blend and compressing it into the desired thickness, then cut into 2.5 mm diameter disc weighing 1 mg each. Various processing aids were investigated in order to improve the cutting process.

### Materials and Methods:

The implant was either a rod (2 mm L×0.72 mm diameter) or a wafer (0.13 mm thickness×2.5 mm diameter) DDS each weighing between 900 μg to 1100 μg. In each formulation Tazarotene was combined with the polymer in a stainless steel mortar and mixed via the Turbula shaker set at 96 RPM for 15 minutes, the powder blend was scraped off the wall of mortar and then remixed for an additional 15 minutes. The mixed powder blend was heated to a semi-molten state at 95° C. for a total of 30 minutes, in three 10-minute intervals, forming a polymer/drug melt. The polymer/drug melt was pelletized using a 9 gauge polytetrafluoroethylene (PTFE) tubing, loaded into the barrel and extruded at the specified core extrusion temperature into filaments, then cut into 1 mg size DDS. Alternatively, the polymer melt was flatten with the Carver press at a specified temperature and cut into 2.5 mm wafers, each weighing 1 mg.

The in-vitro release testing was performed on each lot of implant (DDS or wafer) in three replicates initially, and later in six replicates. Each implant was placed into a 40 mL screw cap vial with 35 mL of saline solution containing various amount of Tween-80 at 37° C. Thirty mL aliquots were removed and replaced with equal volume of fresh medium on day 1, 4, 7, 14, 28, and every two weeks thereafter. The drug assays were performed by HPLC, which consists of a Waters 2690 Separation Module (or 2696), and Waters 2996 Photodiode Array Detector. A Phenomenex Luna C8 (2), 3 μm; 4.6×100 mm column was used for separation and detector was set at 325 nm. The mobile phase was (60:39.8:0.2) acetonitrile-H₂O—CH₃COOH with flow rate of 1 mL/min and a total run time of 15 min per sample. The release rates were determined by calculating the amount of drug being released in a given volume of medium over time in μg/day.

### Results and Discussion:

Initial Dose (100-700 μg) Tazarotene Formulations

The polymers chosen were Boehringer Ingelheim Resomer RG502, RG502H, RG752, and R202H. RG502 is (50:50) poly(D, L-lactide-co-glycolide), RG502H is (50:50) poly(D, L-lactide-co-glycolide) with an acid end group, RG752is (75:25) poly(D, L-lactide-co-glycolide), and R202H is 100% poly(D,L-lactide). All have an inherent viscosity of 0.2 dl/g and can become molten at approximately 90-95° C. The average molecular weight of Resomer RG502, RG502H, RG752 and R202H are 11700, 8400, 11200, and 6500 daltons, respectively.

Preliminary data obtained from the in-vitro release testing of first eight formulations in 0.9% saline showed no drug release for the first two weeks. This was due to the extremely low solubility of Tazarotene in 0.9% saline, which was determined to be less than 1 μg/mL. Therefore, it was difficult to see any differences in the release profiles of different formulations. Work began to search for a release medium that would differentiate the release profiles of all formulations. It was found that the solubility of Tazarotene could be increased by the addition of Tween-80 in 0.9% saline. Comparing the various Tween-80/saline solutions tested (0.25%. 0.5%, 0.75%, and 1%) the 0.5% Tween-80 in saline provided the most stable and predictable release curve, and thus it was used as the medium for all subsequent release testing. The release testing of those eight formulations that showed no release in 0.9% saline was restarted using 0.5% Tween-80 in saline as the release medium.

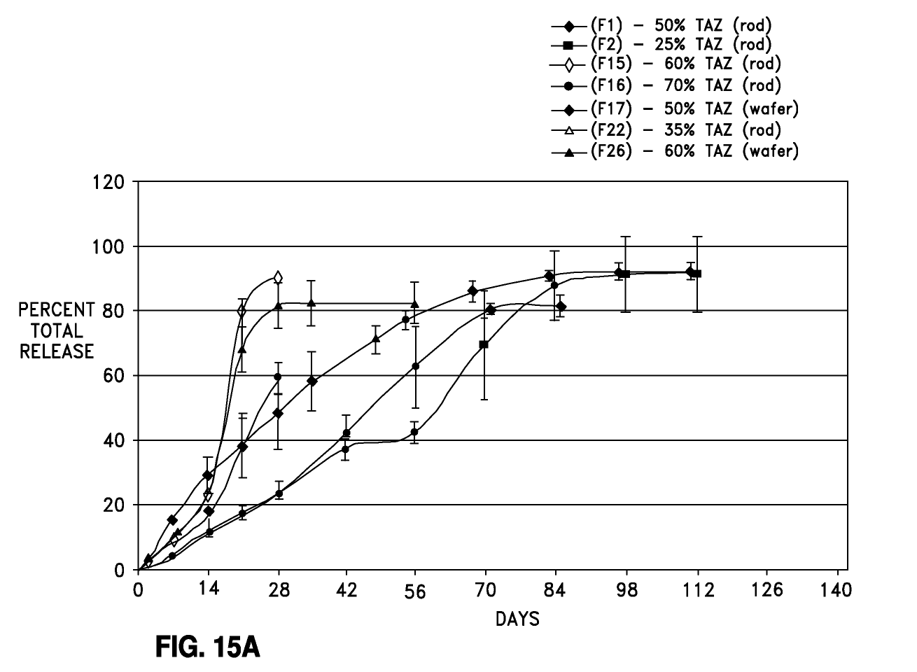

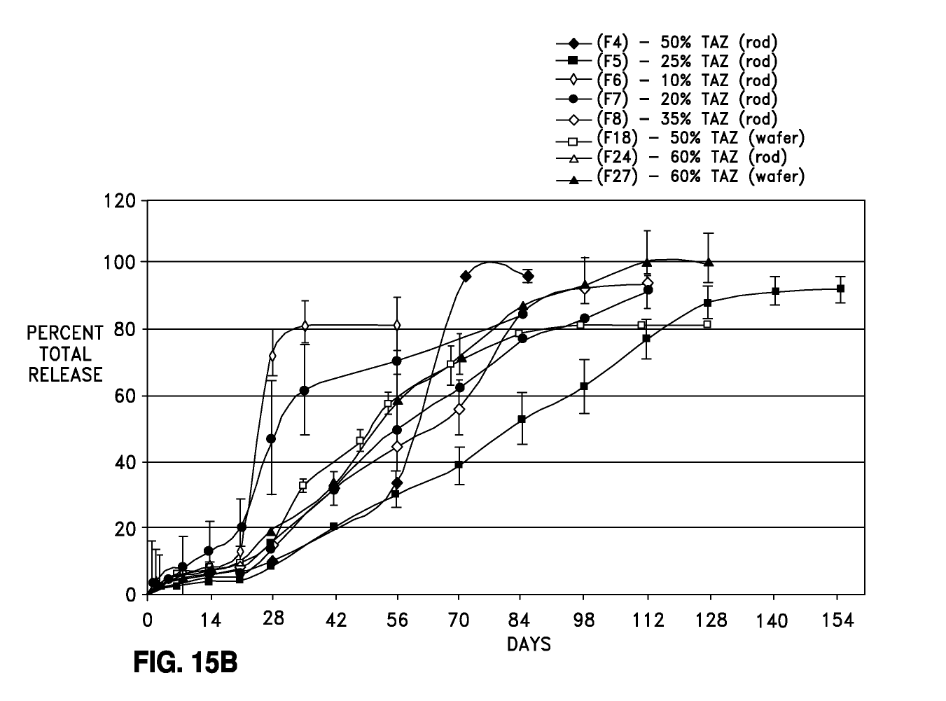

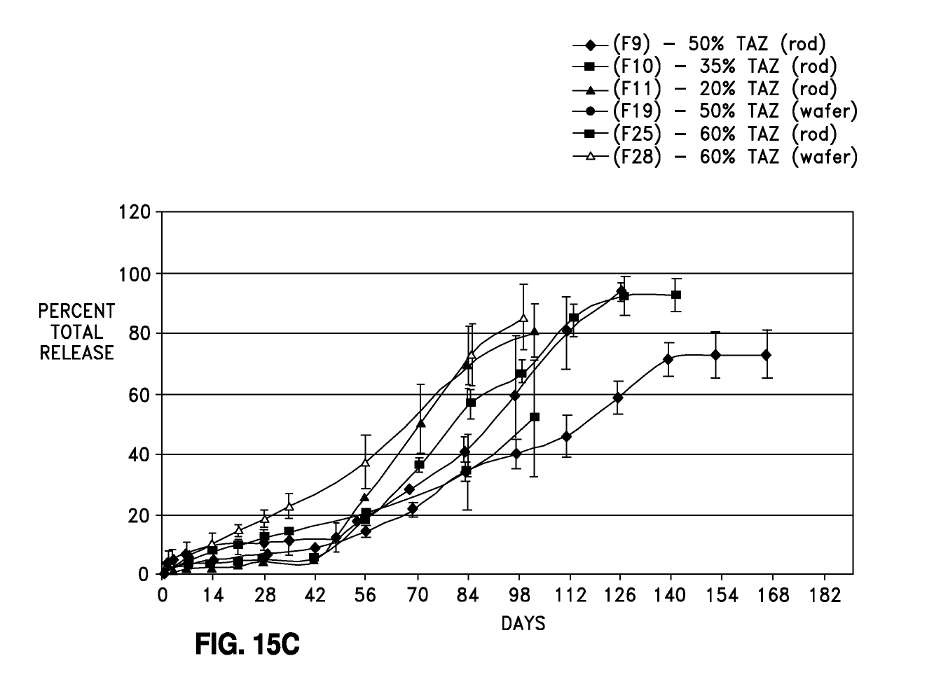

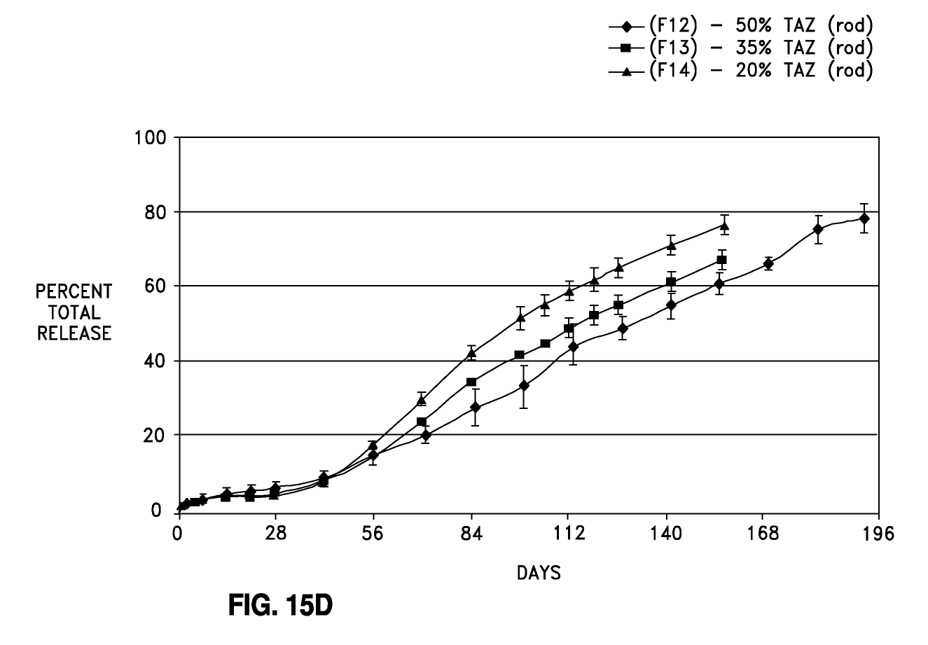

Implants were formulated Tazarotene with the four polymers with varying amounts of drug loads from 10 to 50% (Formulation # 1-14), as shown in Table 5. Based on the release data, other formulations (Formulation # 15-28) were subsequently manufactured with adjustments or modifications. Release data from the first 24 formulations (1-2, 3-19, 22, 24-28) were collected on the designated days and were then compiled based on the polymer, shown in FIGS. 15A, 15B, 15C, and 15D. In theory, there is a general correlation between drug load and release rates; higher drug load yields faster release.

TABLE 5
Tazarotene Formulation (100-700 μg)

| Formulation # | Lot # | Taz | Polymer | I.V. | Extru T | Nozzle | Weight |
|---|---|---|---|---|---|---|---|
| 1 | 295-34 | 50% | RG502H | 0.2 | 87° C. | 720 μm | 1 mg |
| 2 | 295-35 | 25% | RG502H | 0.2 | 81° C. | 720 μm | 1 mg |
| 3 | 295-36 | 10% | RG502H | 0.2 | 78° C. | 720 μm | 1 mg |
| 4 | 295-37 | 50% | RG502 | 0.2 | 91° C. | 720 μm | 1 mg |
| 5 | 295-38 | 25% | RG502 | 0.2 | 84° C. | 720 μm | 1 mg |
| 6 | 295-39 | 10% | RG502 | 0.2 | 80° C. | 720 μm | 1 mg |
| 7 | 295-40 | 20% | RG502 | 0.2 | 84° C. | 720 μm | 1 mg |
| 8 | 295-41 | 35% | RG502 | 0.2 | 88° C. | 720 μm | 1 mg |
| 9 | 295-42 | 50% | RG752 | 0.2 | 86° C. | 720 μm | 1 mg |
| 10 | 295-43 | 35% | RG752 | 0.2 | 82° C. | 720 μm | 1 mg |
| 11 | 295-44 | 20% | RG752 | 0.2 | 79° C. | 720 μm | 1 mg |
| 12 | 295-45 | 50% | R202H | 0.2 | 81° C. | 720 μm | 1 mg |
| 13 | 295-46 | 35% | R202H | 0.2 | 76° C. | 720 μm | 1 mg |
| 14 | 295-47 | 20% | R202H | 0.2 | 74° C. | 720 μm | 1 mg |
| 15 | 295-48 | 60% | RG502H | 0.2 | 91° C. | 720 μm | 1 mg |
| 16 | 295-49 | 70% | RG502H | 0.2 | 97° C. | 720 μm | 1 mg |
| 17 | 295-50 | 50% | RG502H | 0.2 | n/a | wafer | 1 mg |
| 18 | 295-51 | 50% | RG502 | 0.2 | n/a | wafer | 1 mg |
| 19 | 295-52 | 50% | RG752 | 0.2 | n/a | wafer | 1 mg |
| 20 | 295-53 | 50% | R202H | 0.2 | n/a | wafer | 1 mg |
| 21 | 295-54 | 35% | R202H | 0.2 | n/a | wafer | 1 mg |
| 22 | 295-55 | 35% | RG502H | 0.2 | 82° C. | 720 μm | 1 mg |
| 23 | 295-56 | 60% | RG502H | 0.2 | 91° C. | 720 μm | 1 mg |
| (repeat 15) | | | | | | | |
| 24 | 295-57 | 60% | RG502 | 0.2 | 96° C. | 720 μm | 1 mg |
| 25 | 295-58 | 60% | RG752 | 0.2 | 91° C. | 720 μm | 1 mg |
| 26 | 295-59 | 60% | RG502H | 0.2 | n/a | wafer | 1 mg |
| 27 | 295-60 | 60% | RG502 | 0.2 | n/a | wafer | 1 mg |
| 28 | 295-61 | 60% | RG752 | 0.2 | n/a | wafer | 1 mg |
| 29 | 295-62 | 50% | RG502H | 0.2 | 87° C. | 720 μm | 1 mg |
| (repeat 1) | | | | | | | |
| 30 | 295-63 | 50% | RG502H | 0.2 | n/a | wafer | 1 mg |
| (repeat 17) | | | | | | | |
| 31 | 295-66 | Placebo | RG502H | 0.2 | 72° C. | 720 μm | 1 mg |
| 32 | 295-67 | Placebo | RG502H | 0.2 | n/a | wafer | 1 mg |
| 33 | 295-68 | Placebo | R202H | 0.2 | 74° C. | 720 μm | 1 mg |
| 34 | 295-81 | Placebo | RG752 | 0.2 | 79° C. | 720 μm | 1 mg |

We also see different polymers yielding different release rates; RG502H and RG502 yielded a maximum of three to four months release, depending on the drug load, RG752 yielded a maximum of four to six months release, and R202H yielded upwards of six months release and longer. Formulation # 17 (50% Taz/RG502H wafer), formulation # 5 (25% Taz/RG502), formulation # 8 (35% Taz/RG502), formulation # 18 (50% Taz/RG502 wafer), formulation # 9 (50% Taz/RG752), and formulation # 28 (60% Taz/RG752 wafer), all exhibited fairly linear release curves that gave three to six-months release.

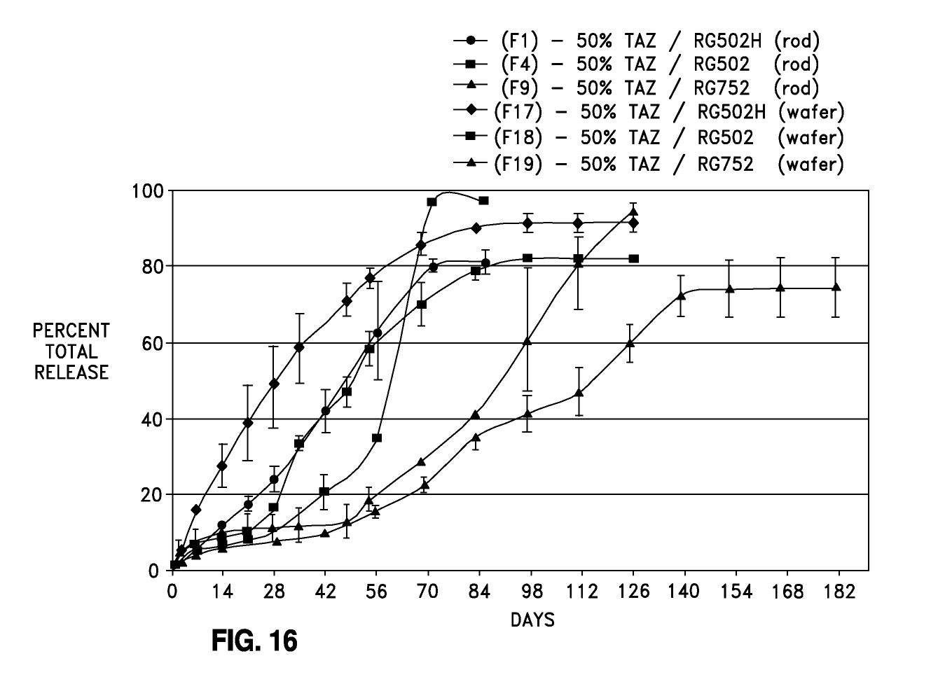

Three rods and three wafers DDSs, were selected to study in-vitro release study using bovine vitreous humor (BVH). The DDS formulations selected were #1, 4, 9, and the wafer formulations selected were #17, 18, and 19. The release profiles of the six formulations are shown in FIG. 16.

Formulation #1 yielded a cumulative Tazarotene release of 80% and leveled off after 71 days, while its wafer counterpart (formulation #17) yielded a cumulative release of 91% and leveled off after 97 days. Formulation #9 yielded a cumulative release of 77% and leveled off after 155 days, while its wafer counterpart (Formulation #19) yielded a cumulative release of 94% after 126 days. Finally, formulation #4 reached its maximum Tazarotene release of 96 after approximately 71 days, while its wafer counterpart (Formulation #18) reached its maximum Tazarotene release of 82% after 97 days.

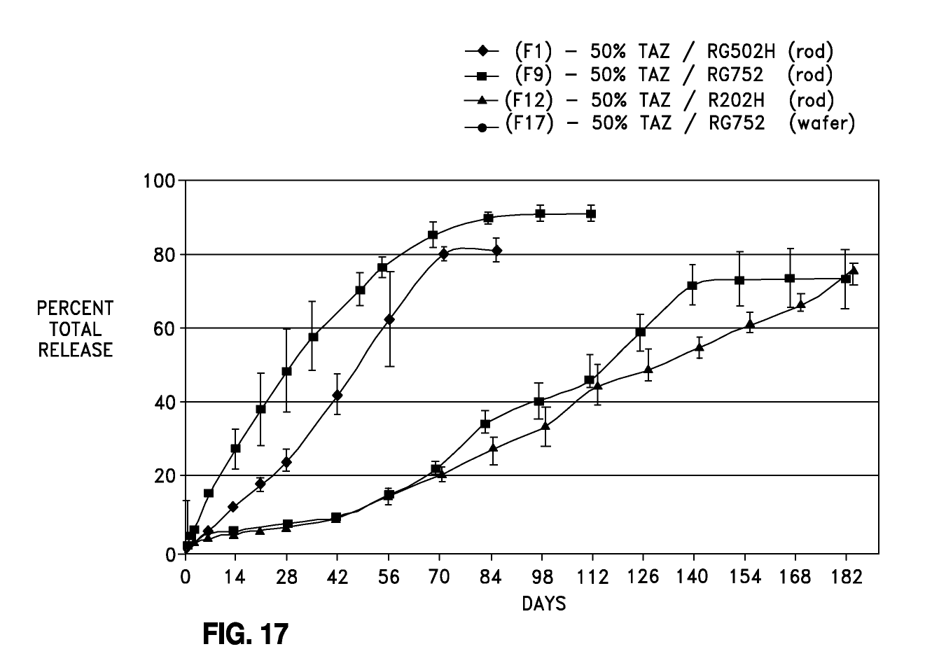

Formulations #1, 9, 12, #17 were studied further. The in-vitro release profiles of these 4 formulations are shown in FIG. 17. Formulation # 9 was studied further Preliminary analysis on product Potency and Content Uniformity before sterilization were 98% and 100.5%±5% of Label Strength, respectively, and product Potency and Content Uniformity after sterilization were 96.6% and 96.9%±3.9% Label Strength, respectively, as shown in Table 6.

TABLE 6
Content Uniformity and Assay of Tazarotene GLP Lot # 229-01

| Content Uniformity and Assay | | | | Content Uniformity and Assay | | | |
|---|---|---|---|---|---|---|---|
| Before Sterilization | | | | After Sterilization | | | |
| Assay | μg/mL | μg/10 dds | % Potency | Assay | μg/mL | μg/10 dds | % Potency |
| Taz-Assay-1 | 49.19 | 4919 | 98.38 | Taz-Assay-1 | 47.88 | 4788 | 95.76 |
| Taz-Assay-2 | 48.76 | 4876 | 97.52 | Taz-Assay-2 | 48.75 | 4875 | 97.50 |
| % Mean | | | 98.0 | % Mean | | | 96.6 |
| Content Uniformity | | | | Content Uniformity | | | |
| Taz-CU-1 | 51.27 | 512.7 | 102.54 | Taz-CU-1 | 45.68 | 456.8 | 91.36 |
| Taz-CU-2 | 50.17 | 501.7 | 100.34 | Taz-CU-2 | 51.00 | 510.0 | 102.00 |
| Taz-CU-3 | 54.20 | 542.0 | 108.40 | Taz-CU-3 | 48.89 | 488.9 | 97.78 |
| Taz-CU-4 | 51.55 | 515.5 | 103.10 | Taz-CU-4 | 49.85 | 498.5 | 99.70 |
| Taz-CU-5 | 46.50 | 465.0 | 93.00 | Taz-CU-5 | 45.42 | 454.2 | 90.84 |
| Taz-CU-6 | 50.71 | 507.1 | 101.42 | Taz-CU-6 | 47.51 | 475.1 | 95.02 |
| Taz-CU-7 | 46.44 | 464.4 | 92.88 | Taz-CU-7 | 49.76 | 497.6 | 99.52 |
| Taz-CU-8 | 51.03 | 510.3 | 102.60 | Taz-CU-8 | 48.27 | 482.7 | 96.54 |
| Taz-CU-9 | 48.27 | 482.7 | 96.54 | Taz-CU-9 | 47.74 | 477.4 | 95.48 |
| Taz-CU-10 | 52.31 | 523.1 | 104.62 | Taz-CU-10 | 50.36 | 503.6 | 100.72 |
| % Mean | | | 100.5 | % Mean | | | 96.9 |
| % RSD | | | 5.0 | % RSD | | | 3.9 |

| | |
|---|---|
| Reference # NB #281, p.168 | Reference # NB #281, p.160 |

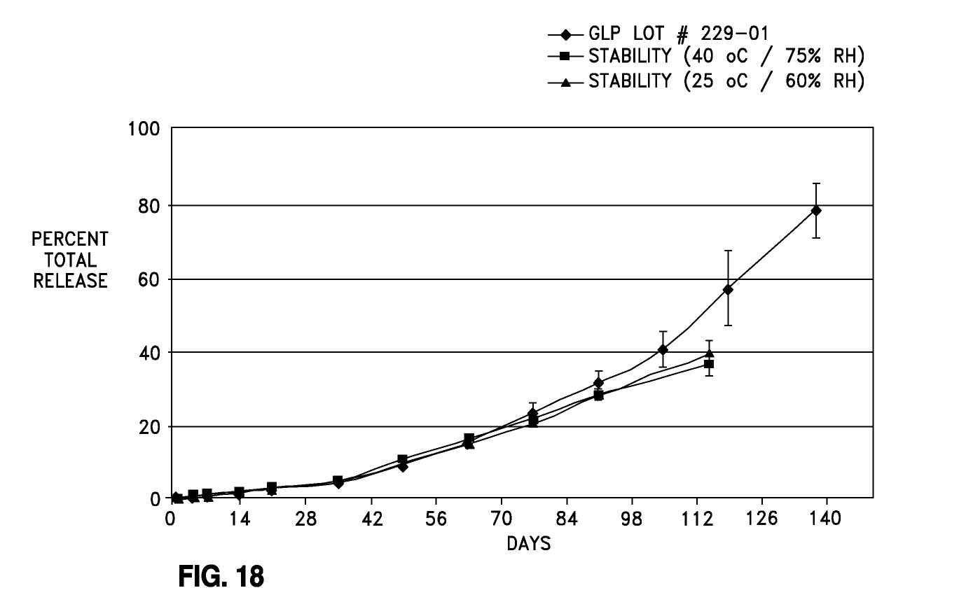

The release profiles of the GLP lot and the stability lots (stored at 40° C./75% RH and 25° C./60% RH for one month) are shown in FIG. 18.

Lower Dose (50 μg) Tazarotene Formulations

Lower dose implants were formulated with 1/10th the dose of Tazarotene (i.e. 50 μg). Since the original implant (Formulation # 9) was 50% Tazarotene in RG752 polymer, the same drug to polymer ratio was used to achieve similar release profiles. Therefore the size of the implant needed to be reduced. In order to accomplish this, the diameter of the filament was reduced from the original 720 μm down to a smaller diameter. The formulations for lower dose Tazarotene implants are shown in Table 7.

TABLE 7
Tazarotene Formulation - Lower Dose (50 μg)

| Formulation # | Lot # | Taz | Polymer | I.V. | Extru T | Nozzle | | Weight |
|---|---|---|---|---|---|---|---|---|
| 47 | 295-96 | 50% | RG752 | 0.2 | 90° C. | 380v | μm | 0.1 mg |
| 48 | 295-97 | 50% | RG752 | 0.2 | 92° C. | 450 | μm | 0.1 mg |
| 49 | 295-98 | 50% | RG752 | 0.2 | 96° C. | 300 | μm | 0.1 mg |
| 50 | 295-99 | 50% | RG752 | 0.2 | 90° C. | 720 | μm | 1 mg |
| 51 | 295-100 | 40% | RG752 | 0.2 | 88° C. | 380v | μm | 0.125 mg |
| 52 | 295-102 | 40% | RG752 | 0.2 | 90° C. | 450 | μm | 0.125 mg |
| 53 | 295-103 | 40% | RG752 | 0.2 | 96° C. | 300 | μm | 0.125 mg |
| 54 | 295-104 | 40% | RG752 | 0.2 | 90° C. | 380 | μm | 0.125 mg |
| 58 | 295-113 | 30% | RG752 | 0.2 | 94° C. | 300 | μm | 0.16 mg |
| 59 | 295-114 | 30% | RG752 | 0.2 | 88° C. | 450 | μm | 0.16 mg |
| 60 | 295-115 | 30% | RG752 | 0.2 | 88° C. | 380 | μm | 0.16 mg |
| 61 | 295-116 | 30% | RG752 | 0.2 | 88° C. | 380v | μm | 0.16 mg |
| 62 | 295-117 | 25% | RG752 | 0.2 | 88° C. | 380 | μm | 0.2 mg |
| 63 | 295-118 | 25% | RG752 | 0.2 | 89° C. | 380v | μm | 0.2 mg |
| 64 | 295-119 | 25% | RG752 | 0.2 | 89° C. | 450 | μm | 0.2 mg |
| 65 | 295-120 | 25% | RG752 | 0.2 | 94° C. | 300 | μm | 0.2 mg |

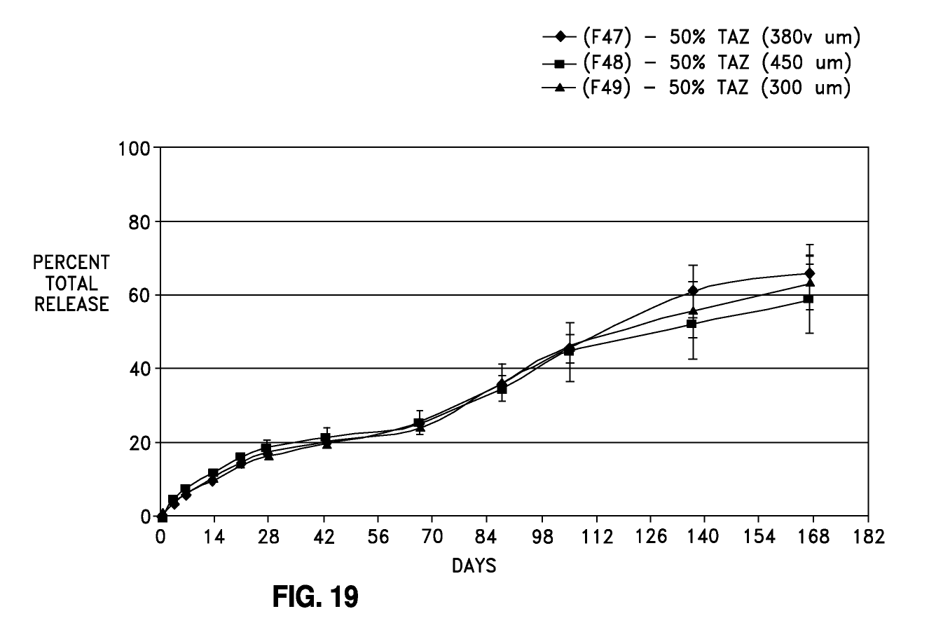

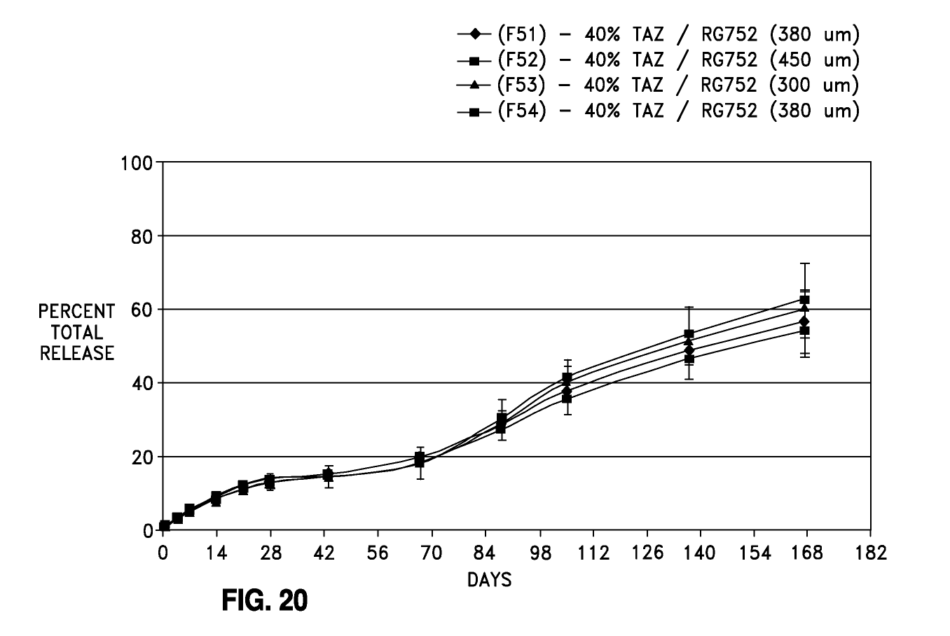

For these implants, four different nozzle diameters were used, 300 μm, 380 μm, 380v μm, and 450 μm. The difference between 380 and 380v is that the inlet of the former has a shallow groove and the latter has a v-groove. Release data taken showed that within the same drug load, there were no significant differences among the DDS prepared with 300 μm, 380 μm, 380v μm, or 450 μm diameter nozzles, as shown in FIG. 19 and FIG. 20.

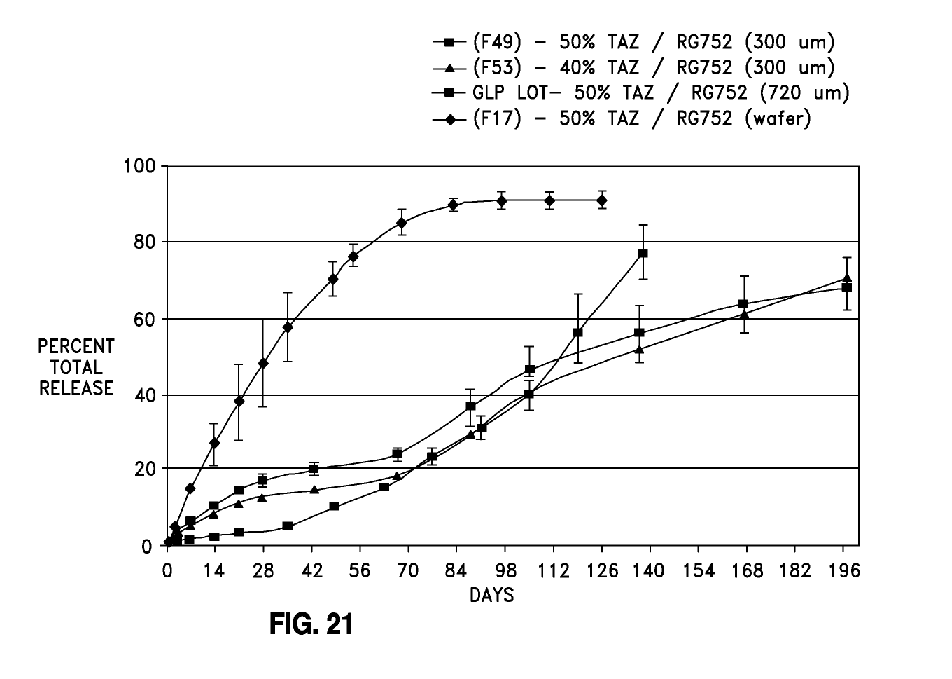

Formulations #49 and #53 and their corresponding placebos, and one regular dose wafer formulation, #17 (wafer, 1 mg) plus its placebo were studied further. Formulation #49 was 50% Taz/RG752 with filament diameter of 300 μm, and formulation #53 was 40% Taz/RG752 also with filament diameter of 300 μm. As shown in FIG. 21, both formulations have release rate slightly faster than the GLP lot up to day 105 releasing 46% and 40% for #49 and #53, respectively, while the GLP lot released 40%. On day 137, formulation #49 and formulation #53 were releasing 56% and 52%, respectively, while the GLP lot was releasing 77% at approximately the same time. In contrast, formulation # 17 was by far the fastest of all four, releasing up to 91% after 97 days.

Additional 500 μg Tazarotene Formulation: Linear Release Profile

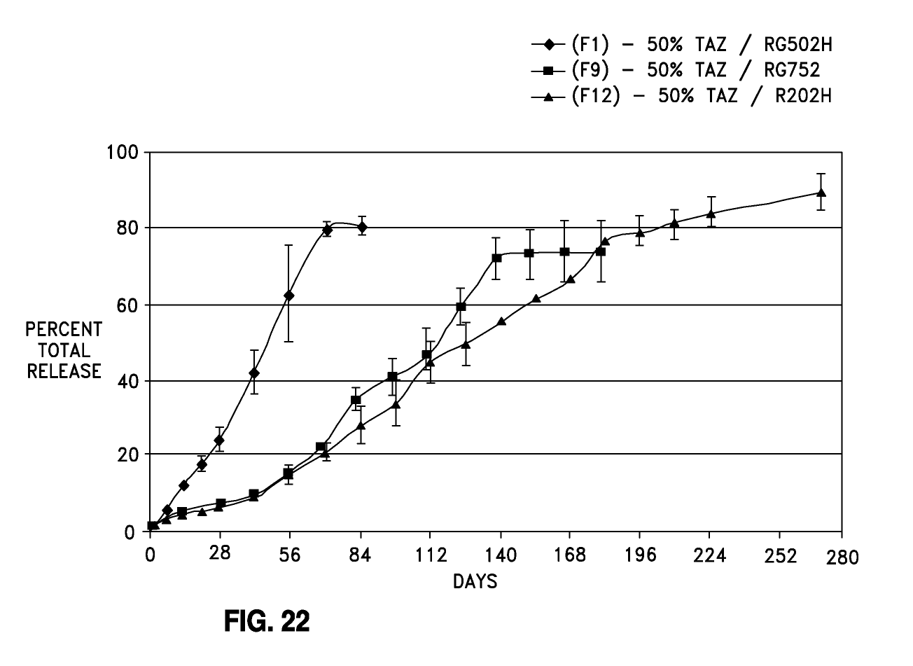

After the completion of the GLP lot of formulation #9, an effort was started to formulate Tazarotene with more linear release profiles. One approach was to combine two different polymers with Tazarotene. The selection of the two polymers was meant to compliment the release profile of Tazarotene with each individual polymer. An example of a potential blend was to use polymers RG502H and R202H, and RG752. Their individual release profile is shown in FIG. 22. Tazarotene formulated with RG502H reached a maximum of 81% after 85 days, while Tazarotene formulated with RG752 reached a maximum of 74% after 181 days, and Tazarotene formulated with R202H was much slower and it reached 90% after 269 days.

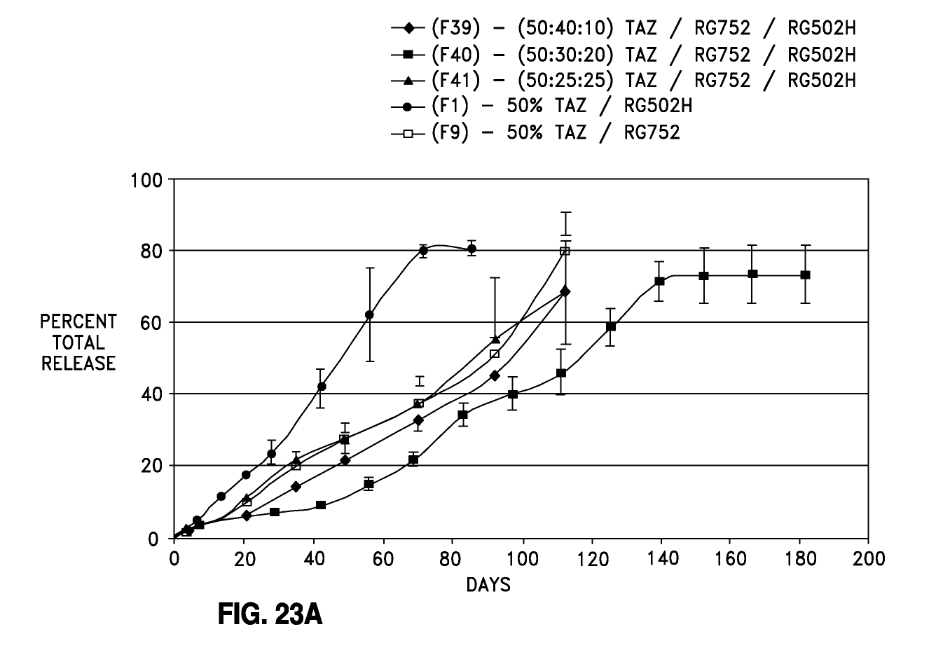

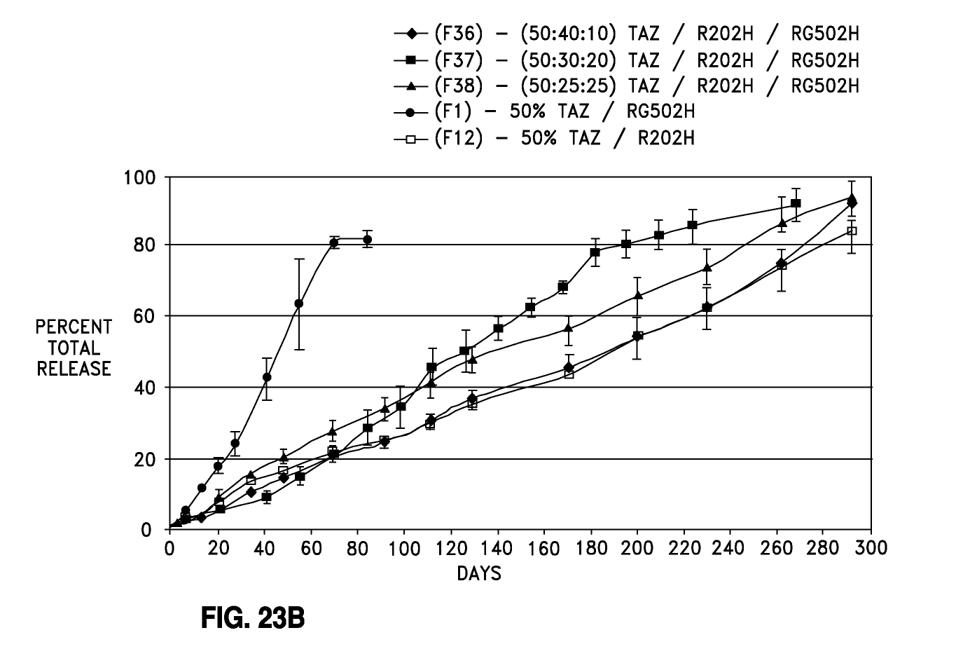

Three different blends of Tazarotene/R202H/RG502H with the ratio of (50:40:10), (50:30:20), and (50:25:25) were made. Likewise, three different blends of Tazarotene/RG752/RG502H with the ratio of (50:40:10), (50:30:20), and (50:25:25) were made. The release profiles are shown in FIGS. 23A and 23B.

In FIG. 23A, the graph clearly showed that the release profiles of the three different blends of RG752 and RG502H were in between the release profile of Taz with RG752 and that of Taz with RG502H, as expected. Furthermore, up to day 100, the blend containing more RG502H released at a faster rate than the blend containing less RG502H. In FIG. 23B, the release profiles of the blends were in between the release profiles of Taz with R202H and Taz with RG502, up to day 60, and then the profiles of the blend continued their release at near zero order kinetics. Again, the blend with more RG502H released at a faster rate. It is interesting to note that linearity was achieved using RG502H and R202H, which are PLGA and PLA, respectively. The combination of RG502H and RG752 was not as successful at obtaining a more linear profile considering the release profiles of formulation #9 and formulation #12 were somewhat similar.

Additional Wafer Formulations: Addition of Processing Aids.

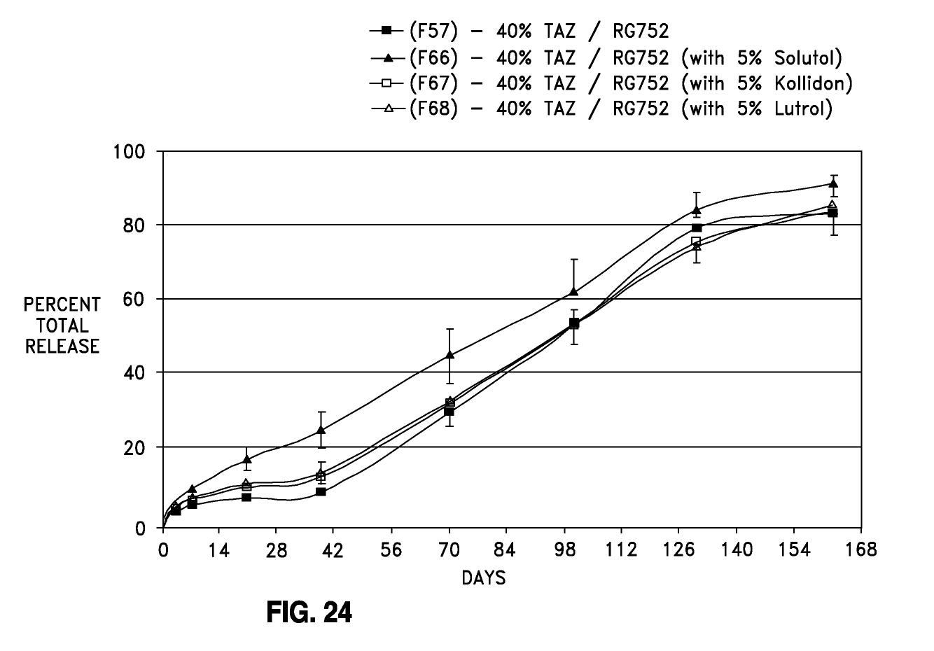

During fabrication of Tazarotene wafers, it was noticed that some formulations (#20 and #21) were too fragile or brittle to process (each wafer has a thickness of 0.005 inch or 0.127 mm and a diameter of 2.5 mm), while the others could be made but with losses up to 50% or more. Therefore, we decided to use processing aids that can change the mechanical properties of the wafers, making them less brittle. Solutol®, Kollidon®, and Lutrol®, excipients commonly used in many oral dosage forms, were our first 3 candidates. Solutol is polyethylene glycol 660 12-hydroxystearate, Kollidon (12 or 17) is polyvinyl-pyrrolidone, and Lutrol is a copolymer of polyethylene glycol and polypropylene glycol. All three additives when added to Tazarotene formulations led to wafers that were less brittle, much easier to manufacture and with higher yields. The release profiles of wafers made with these additives are shown in FIG. 24.

Similar release profiles were obtained for Formulation 67 and formulation 68 when compared to formulation #57, while formulation #66 showed a more linear and slightly faster release profile.

### CONCLUSIONS

Over 30 different Taz formulations were prepared using low inherent viscosity poly (D,L-lactide-co-glycolide)/poly (D,L-lactide) polymers. Their release profiles were monitored in 0.5% Tween-80 in saline at 37° C. Tazarotene (Formulation # 9, 50:50 Taz/RG752), with Resomer RG752 poly (lactide-co-glycolide) from Boehringer Ingelheim provided close to six months in-vitro continuous drug release profile. The Tazarotene implants weighed approximately 1 mg each and contained 500 μg of active pharmaceutical ingredient. The implants passed the Potency (96.6%) and Content Uniformity (96.9%±3.9%) specifications and have good stability. Implants with one-tenth dosage as the original formulation (implant with 50 μg of API), new Tazarotene formulations with a more linear release than formulation #9, as well as Tazarotene wafers with additives as processing aids were also manufactured and studied.

### Example 11

### Retinoid-Containing Intraocular Implants and Proliferative Vitreoretinopathy

PLA and PLGA implants 1.5 mm in diameter and 3 mm in length were fabricated by extrusion of the polymer drug blend. Tazarotenic acid (the free acid of Tazarotene) Tazarotene and 13-cis-retinoic acid were loaded into the implants at 10% concentrations. Briefly, pigmented rabbits were vitrectomized followed by intravitreal injection of 500,000 human RPE cells. After injection of the RPE cells the retinoid implants were placed into the vitreous and anchored to the sclera with the suture used to close the sclerotomy.

The eyes were examined weekly for 4 weeks. Severity of PVR was graded based on the Fastenberg scale. At 4 weeks gross pathology and limited histopathology were conducted. At 28 days only 12% of the Tazarotene and Tazarotenic acid treated eyes progressed to traction retinal detachment and 22% of the accutane treated eyes progressed. This is in comparison to 94% of the control eyes experiencing stage 3 or greater tractional detachment. This study clearly demonstrated the efficacy of the Tazarotene implants in this animal model. Gross pathology and limited histopathology has shown a good safety profile for Tazarotene and its PLGA/PLA implant.

Tazarotenic Acid Ocular Concentrations

TABLE 8
Tazarotene and Tazarotenic Acid plasma concentrations
after topical and systemic delivery.

| | | | TAZAROTENIC | |
|---|---|---|---|---|
| | | TAZAROTENE | ACID | |
| Route | Dose (Conc.) | Cmax (ng/mL) | Cmax (ng/mL) | Study |
| Topical Gel | 0.1% 2% BSA | Tazarotene | 0.241 | R-168-155-8757 |
| | 0.1% 7% BSA | concentrations | 0.83 | R190168-022 |
| | 0.1% 15% BSA | BLQ for >90% | 4.80 | R168-152-8606 |
| | 0.1% 20% BSA | | 12.0 | R-168-153-8606 |
| | 0.1% Phase 3 | | 90% <1 ng/mL | |
| Oral | 1.1 mg/Day | 95% <0.1 ng/mL | 28.9 | 190168-018P-00 |
| Oral | 3.0 mg/Day | <0.1 ng/mL | 81.6 | 190168-019P |
| Oral | 6.0 mg/Day | <0.1 ng/mL | Cₘₐₓ 227 | 190168-044P |
| | | | C_{trough} 2.56 | |

Table 8 summarizes the vitreous retinoid levels after topical and oral dosing of tazarotene. For the most part tazarotene is not observed in the plasma after topical or oral administration. Facile hydrolysis by pre-systemic metabolism rapidly generates the free acid. Tazarotenic acid plasma concentrations (Cmax, maximal plasma level) from topical administration range from 0.25 ng/mL to 12 ng/mL. It is important to note that these are plasma concentrations and the eye plasma distribution ratio is 0.02. Over 90% of all patients in the phase 3 clinical trials had concentrations of the parent compound, tazarotene, <1 ng/mL with the highest being 6 ng/mL.

Oral delivery of 1.1 mg and 6 mg multiple doses led to 28.9 ng/mL and 227 ng/ml peak levels with a 2.56 ng/mL trough. This corresponds to a maximum possible 4 ng/mL ocular level for the highest dose. FIG. 11 depicts the tissue distribution of tazarotenic acid. The eyes show a 2% tissue/plasma ration in the rat. It should be noted that tazarotenic acid is 99% protein bound and distribution is limited to unbound drug. Hence, the overwhelming majority of ocular tazarotenic acid concentrations are most probably in the anterior tissues. Further, the log P of tazarotenic acid is calculated to be 2.53 hence it is not expected to display efficient penetration of the blood-retinal barriers.

Subconjunctival Microspheres

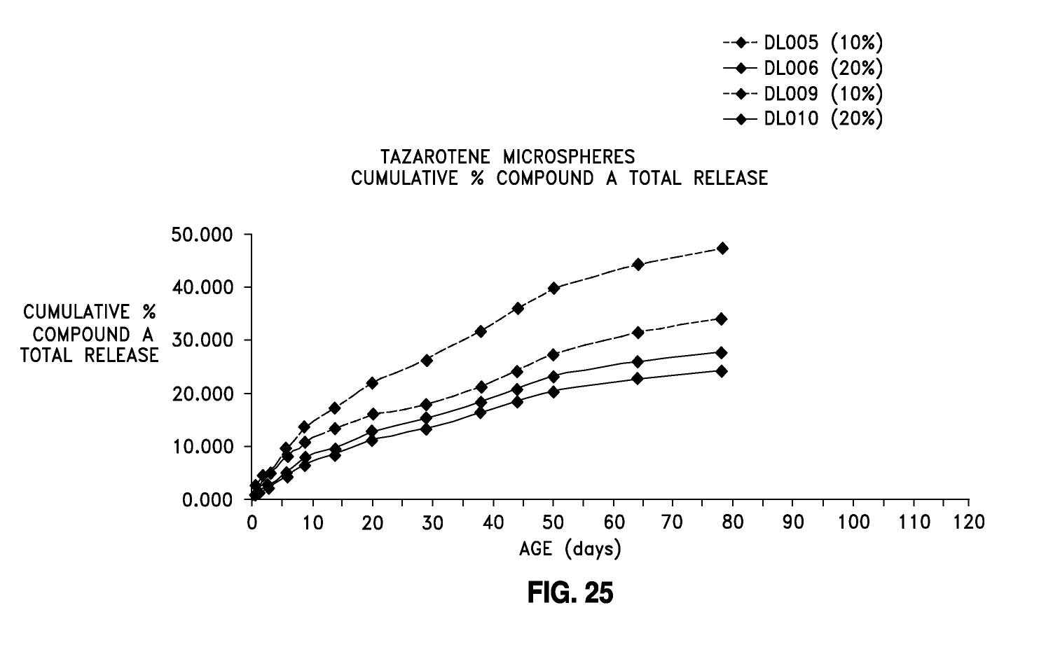

Tazarotene PLGA microspheres loaded with 20% tazarotene were also administered subconjunctivally at a dose of 2 mg. The specific formulation tested is a 75:25 PLGA microsphere (Applied Polymer Technologies PLGA 75:25 inherent viscosity 0.67 dl/gm, 20% tazarotene/80% polymer, dose 2 mg). The release profile of these microspheres is depicted in FIG. 25.

The intraocular pharmacokinetics of Tazarotene was assessed in female New Zealand White rabbits following a single intravitreal injection. The rabbits were dosed with bilateral intravitreal injections of Tazarotene (1250 ng in 50 μL). At 0.5, 1, 2, 4, 8, 12, and 24 hr post dose, animals were sacrificed and the aqueous humor, vitreous humor, and retina samples were analyzed. The Tazarotene concentration in the vitreous humor declined from 578±77 ng/g at 2 hr post dose to 115±33 ng/g by 24 hr post dose with a mean half-life (t_{1/2}) of 9.22 hr. The mean vitreal clearance (Cl) was estimated to be 0.123 mL/hr. The Tazarotene concentration in the retina was close to its vitreal concentration at all time points, declining from 859±131 ng/g at 2 hr post dose to 93.1±28.9 ng/g by 24 hr post dose. Elimination of Tazarotene from the retina had a similar mean t_{1/2} of 7.63. It was shown that Tazarotene has a relatively long intravitreal half-life and that the retinal concentration parallel the vitreous when given as a neat injection. However, despite a very low solubility the clearance of Tazarotene from the retina is fast enough to require sustained release for extended duration of action.

The pharmacokinetics of sustained release tazarotene implants was assessed. Based on the preliminary data of intravitreally administered tazarotene a dose of 500 mg delivered over 6 months was chosen for sustained release. The vitreal clearance of tazarotene is 2.95 mL/day, hence the desired release rate over the 6 month period of time is 3 μg/day. Formulations 1 (F1), 9 (F9) and 12 (F12) have release rates of 1.8, 2.5 and 5.5 mg/day, respectively (See FIG. 16). Formulation 17 (F17) releases at a higher rate of 7 μg/day as it is implanted subconjunctivally and must first penetrate the RPE to access the eye (see FIG. 16).

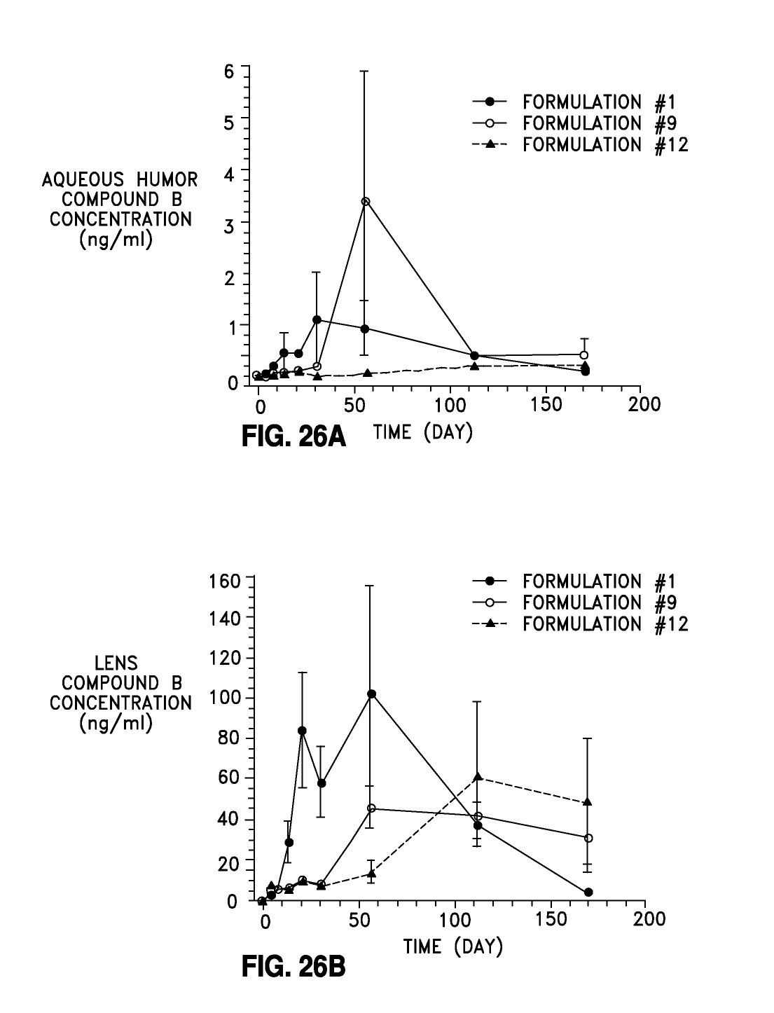

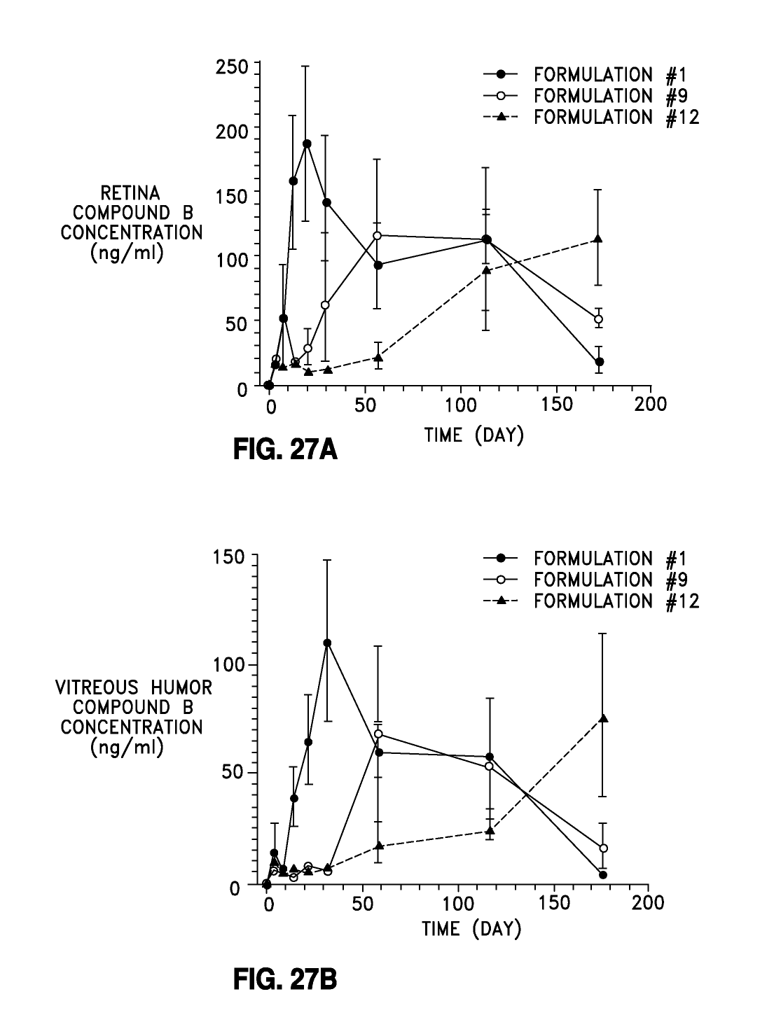

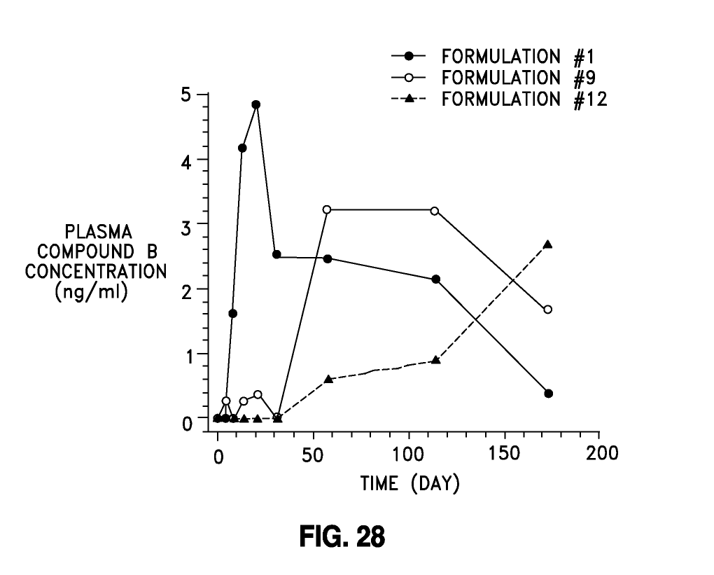

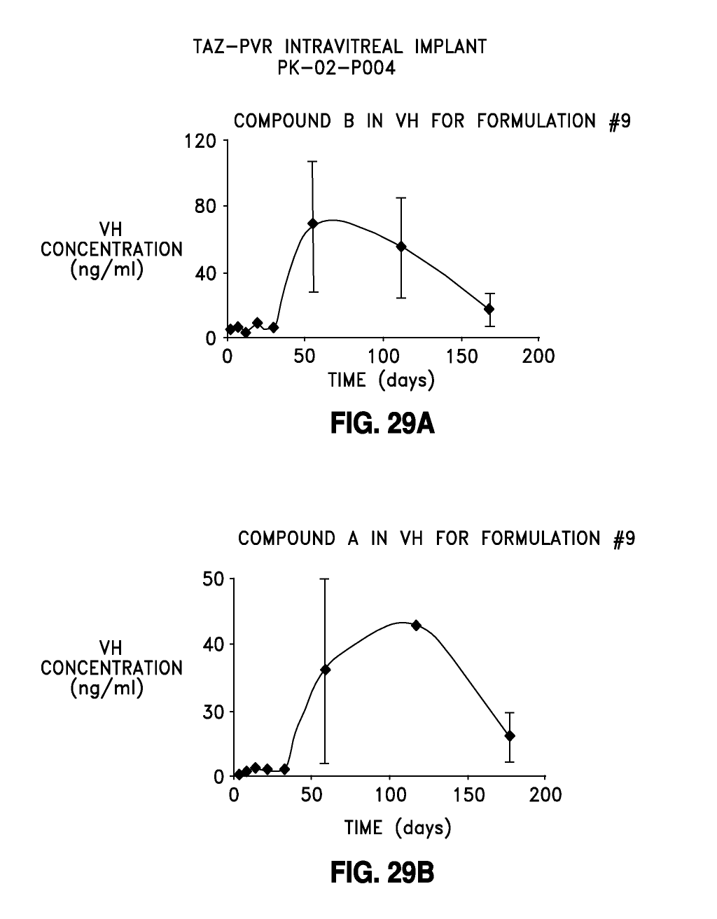

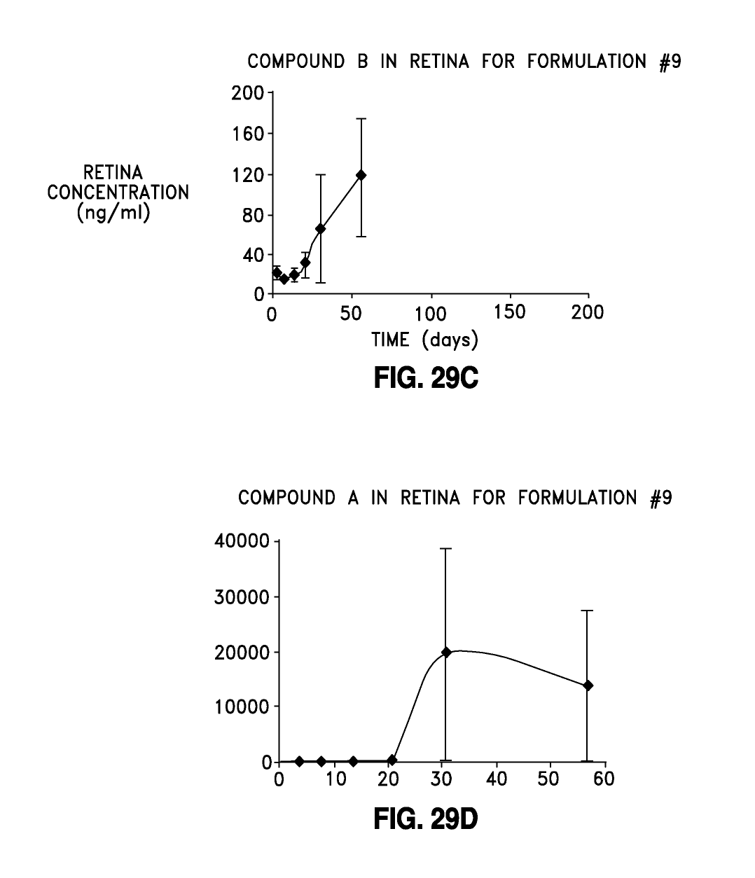

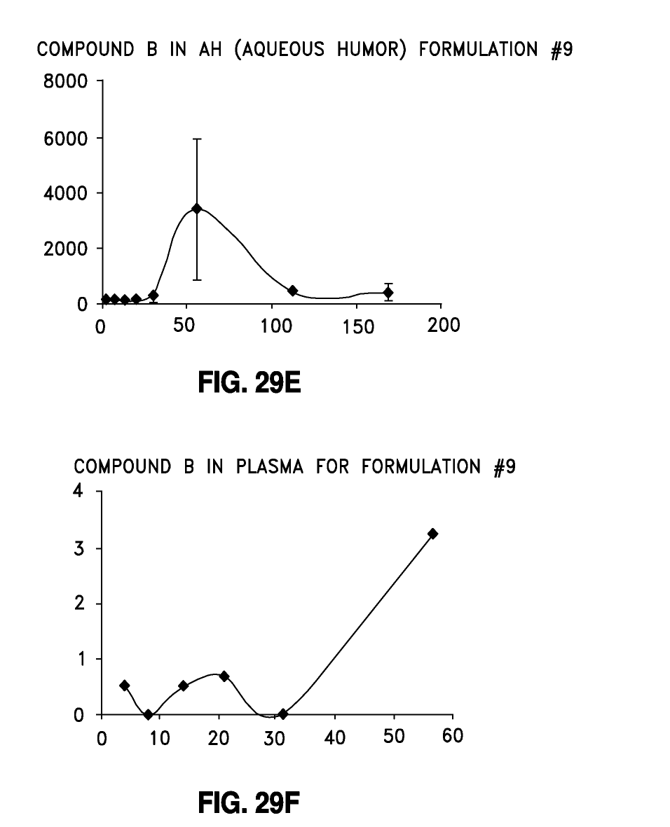

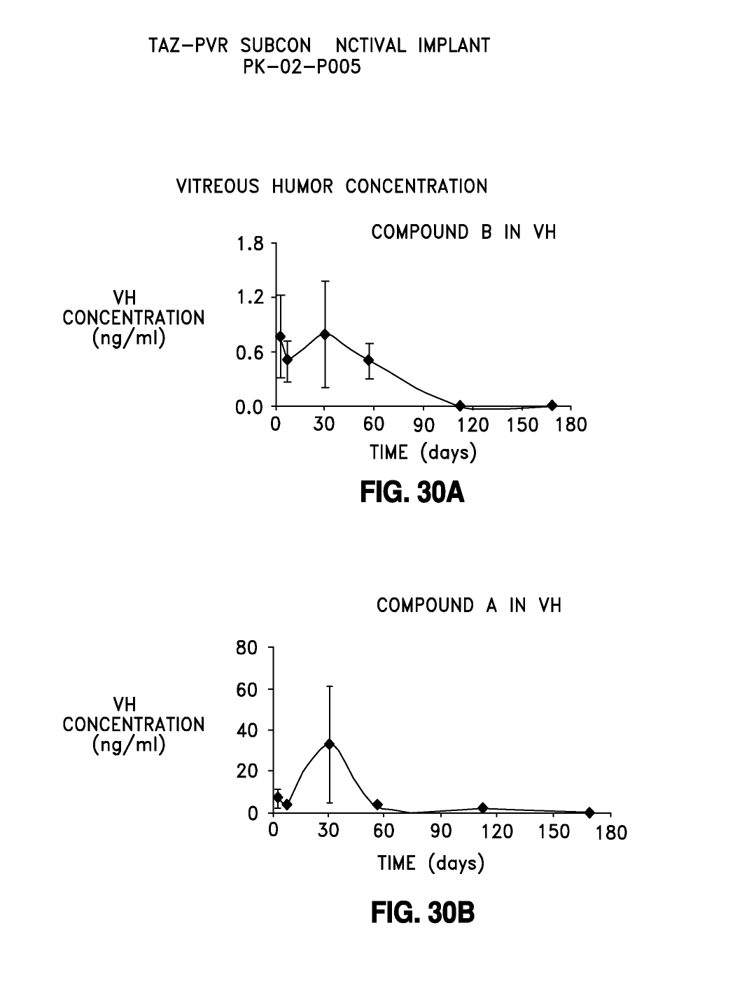

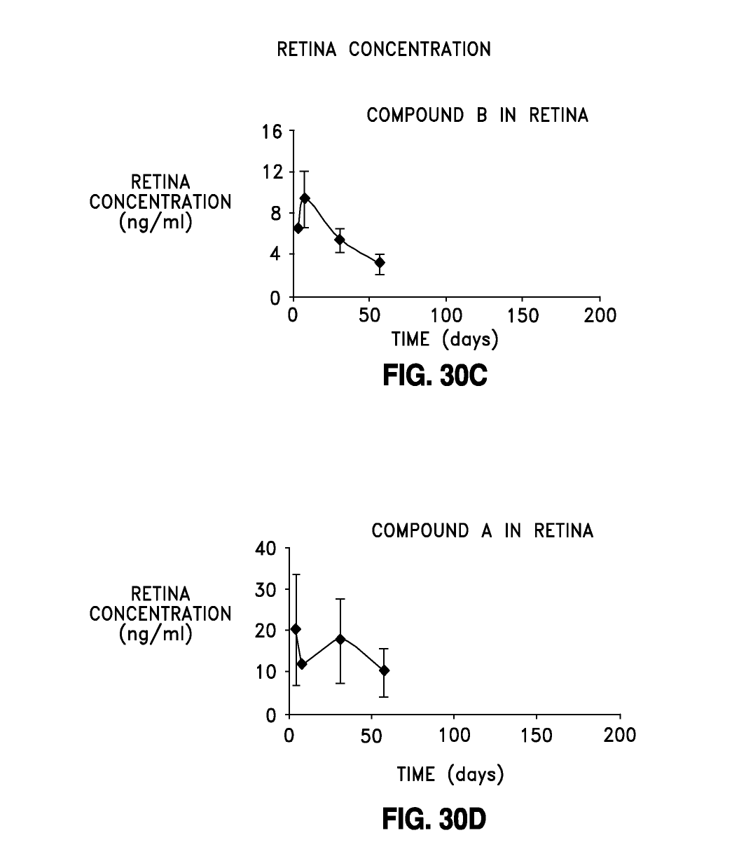

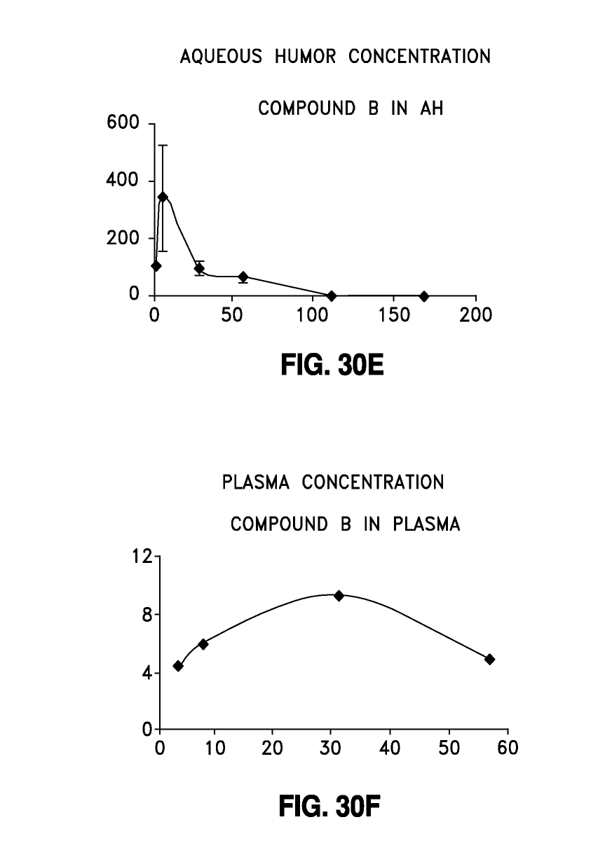

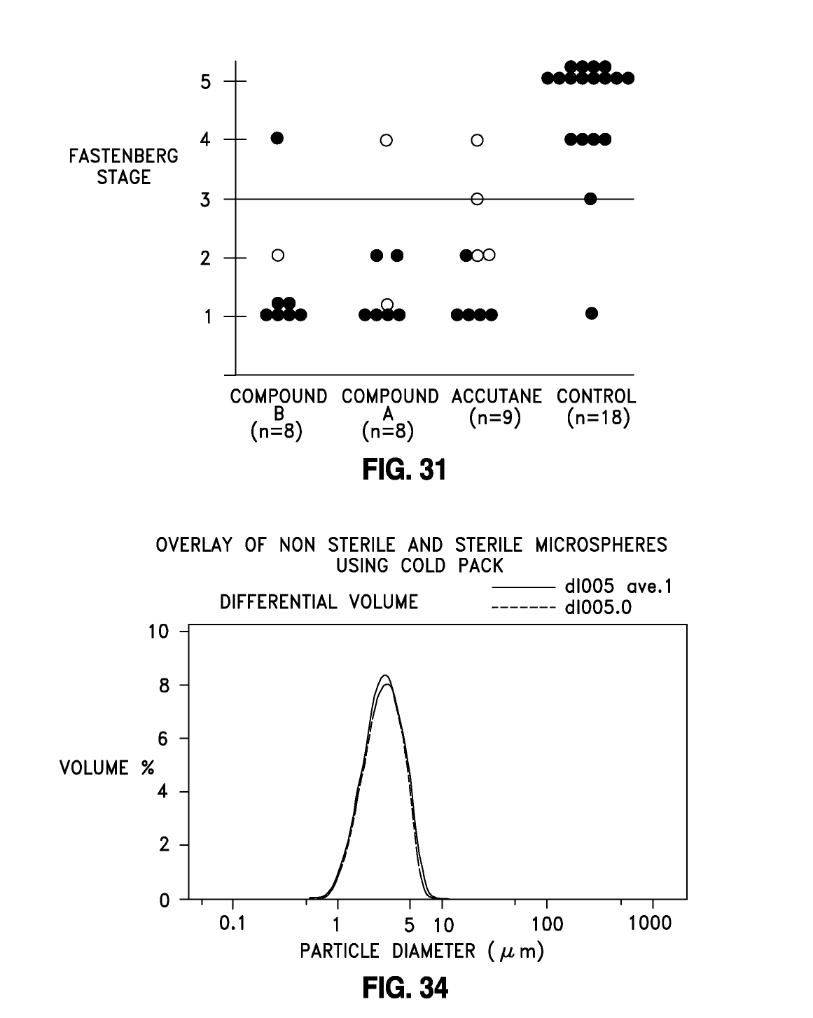

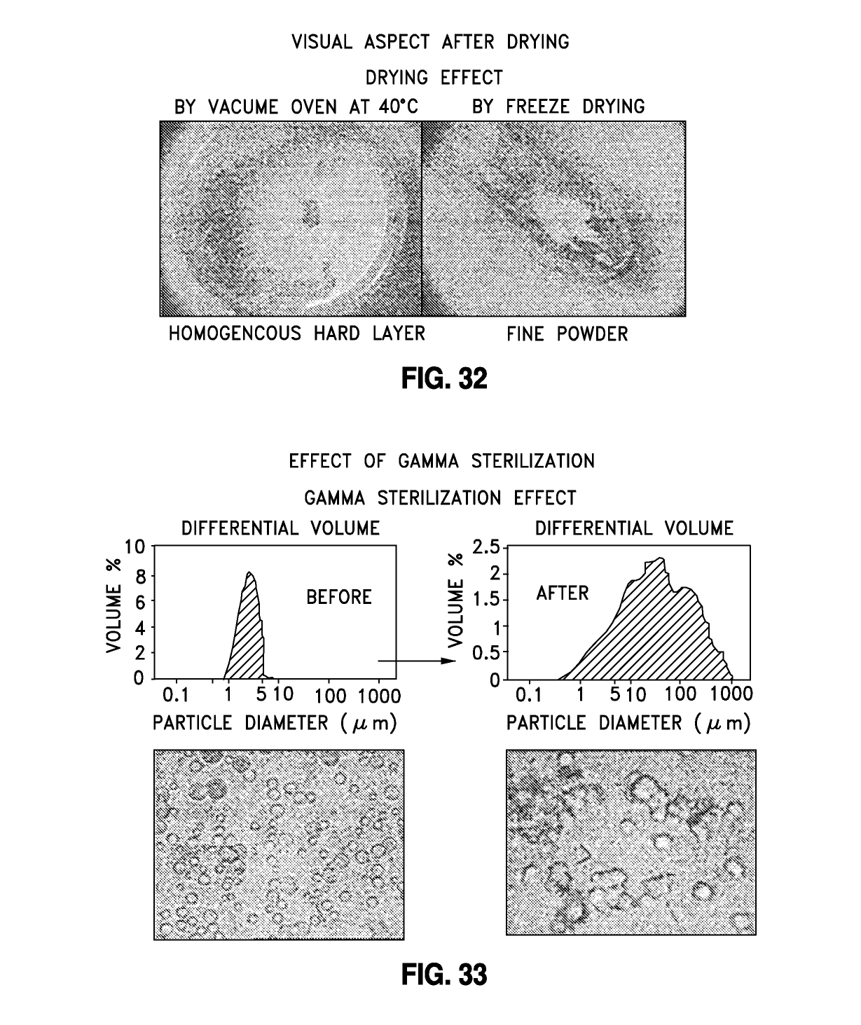

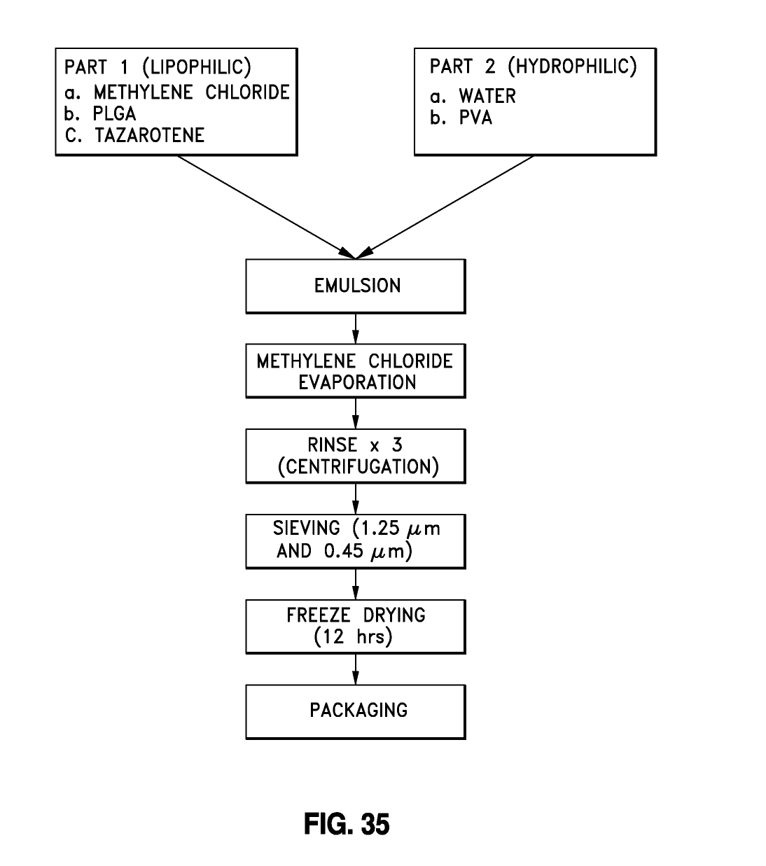

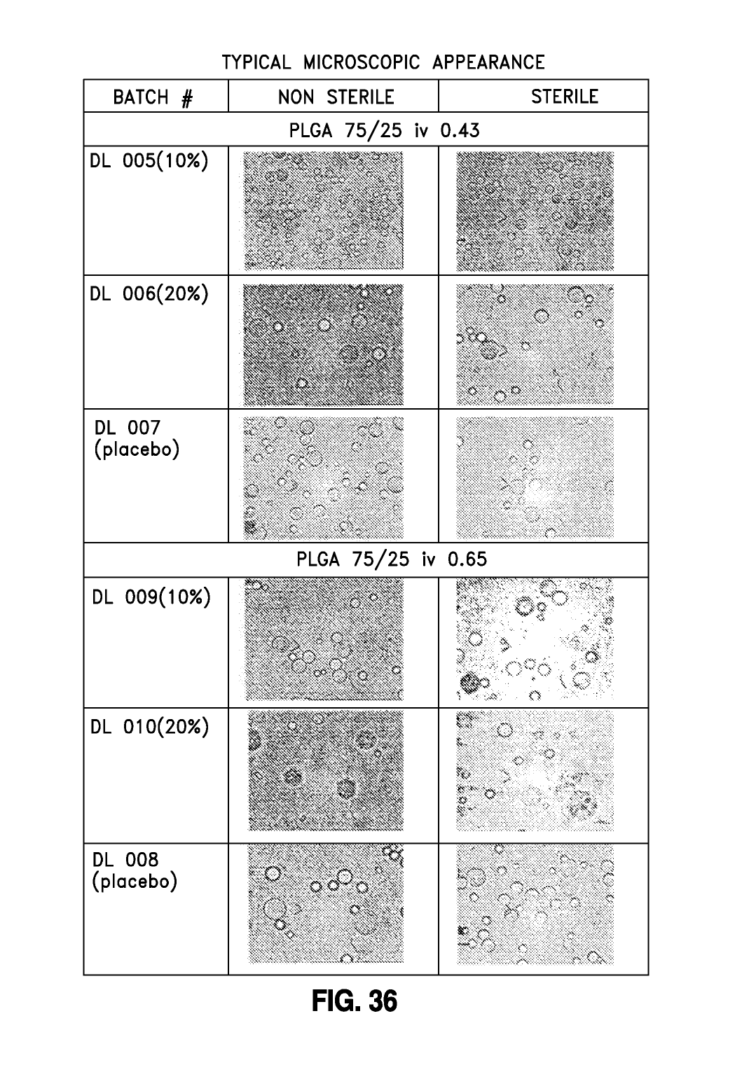

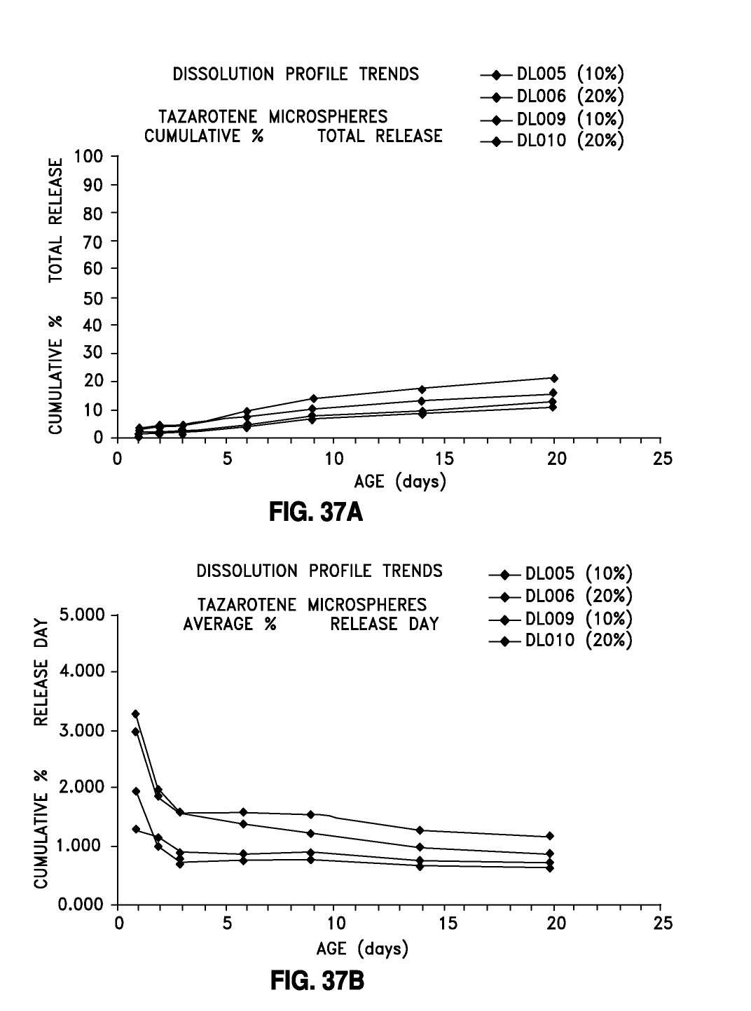

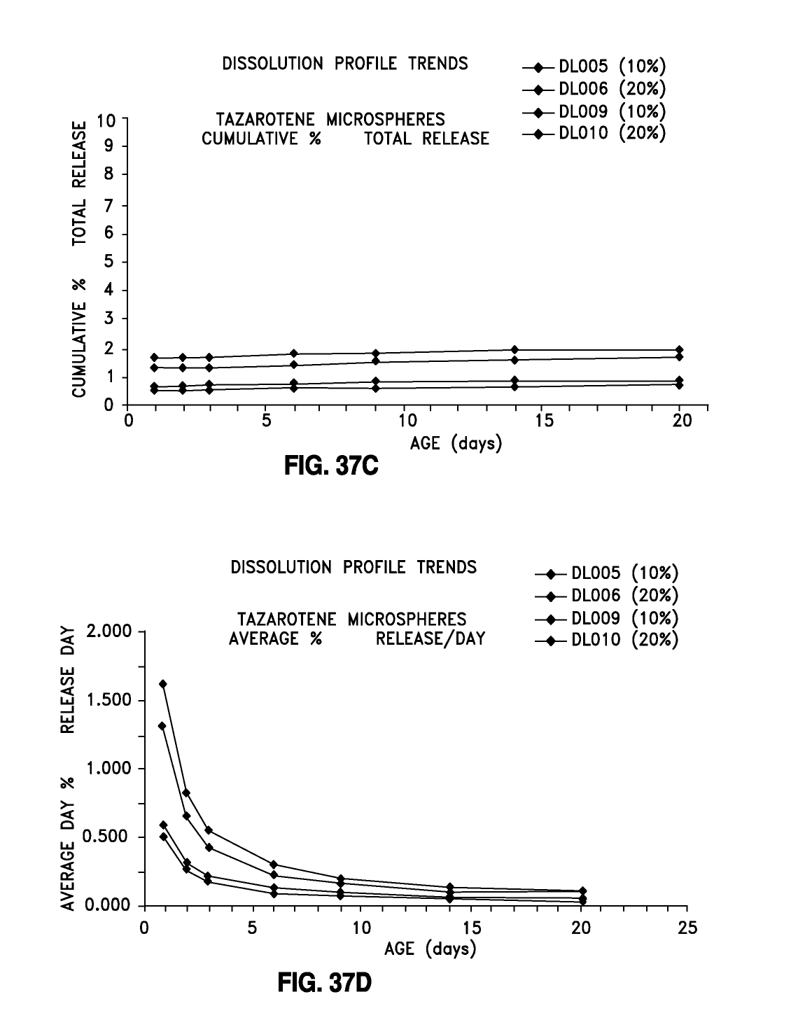

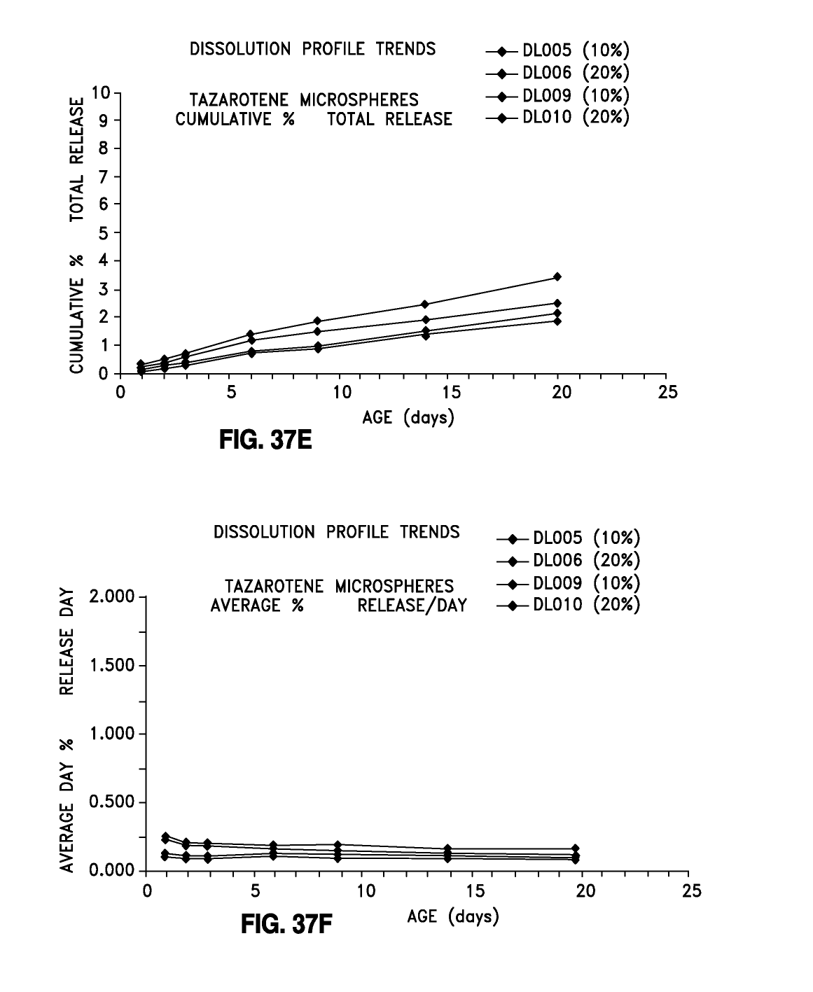

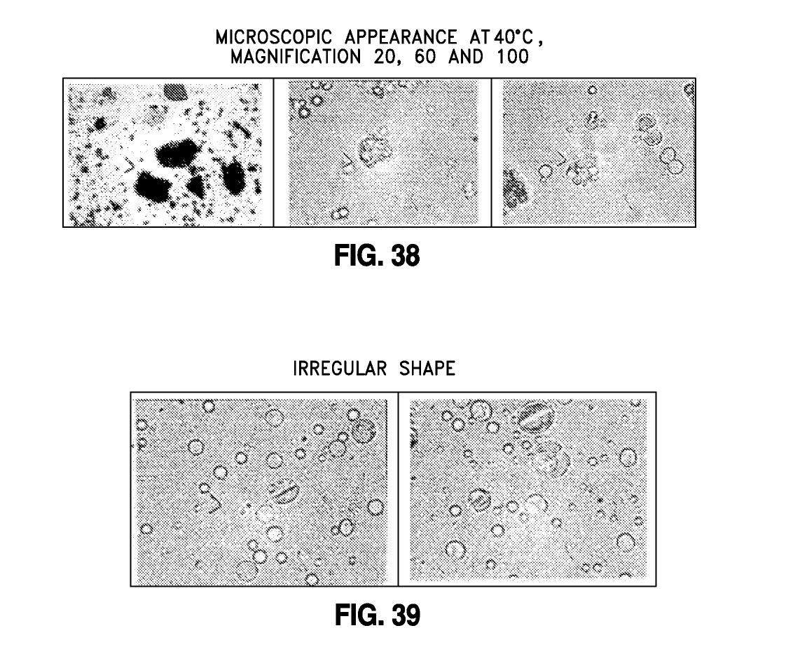

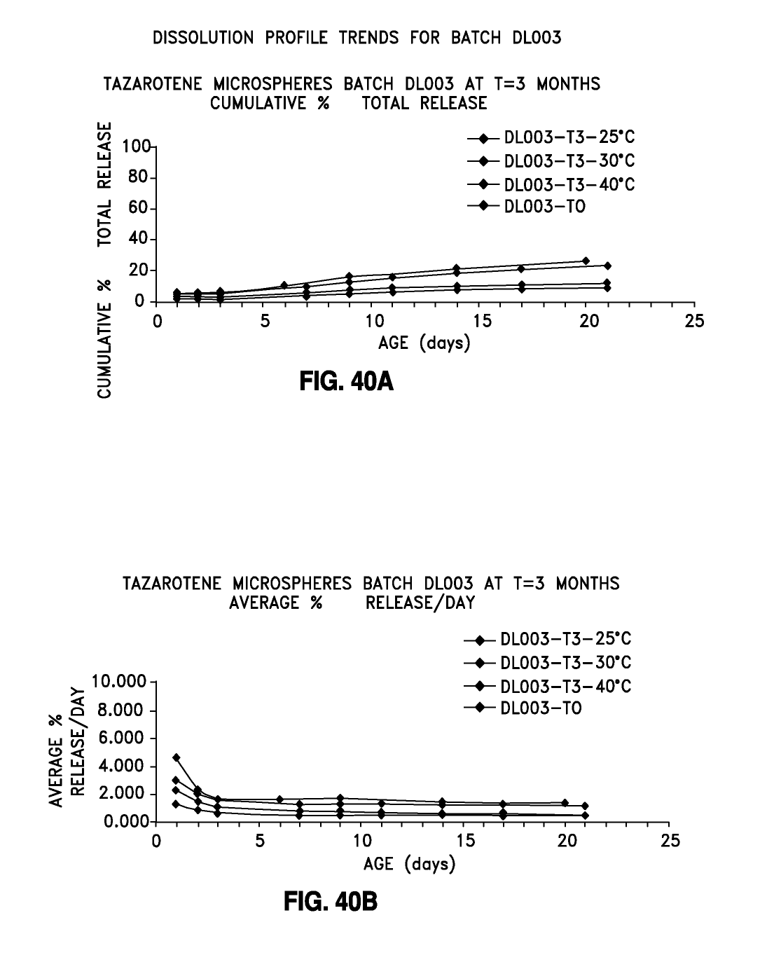

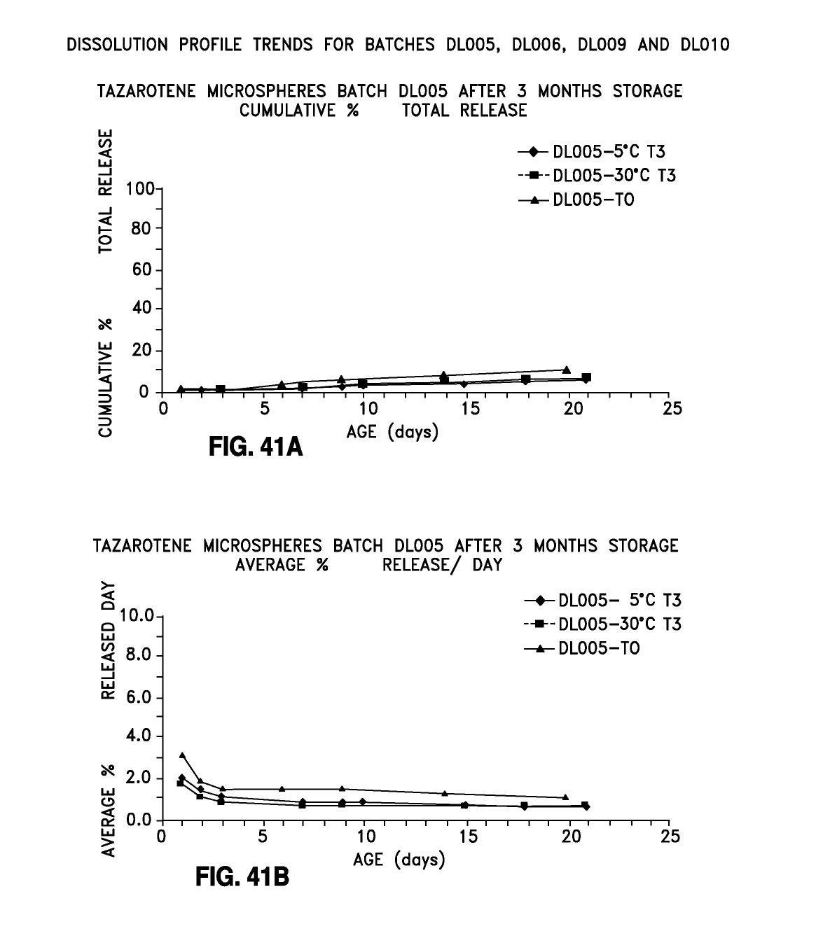

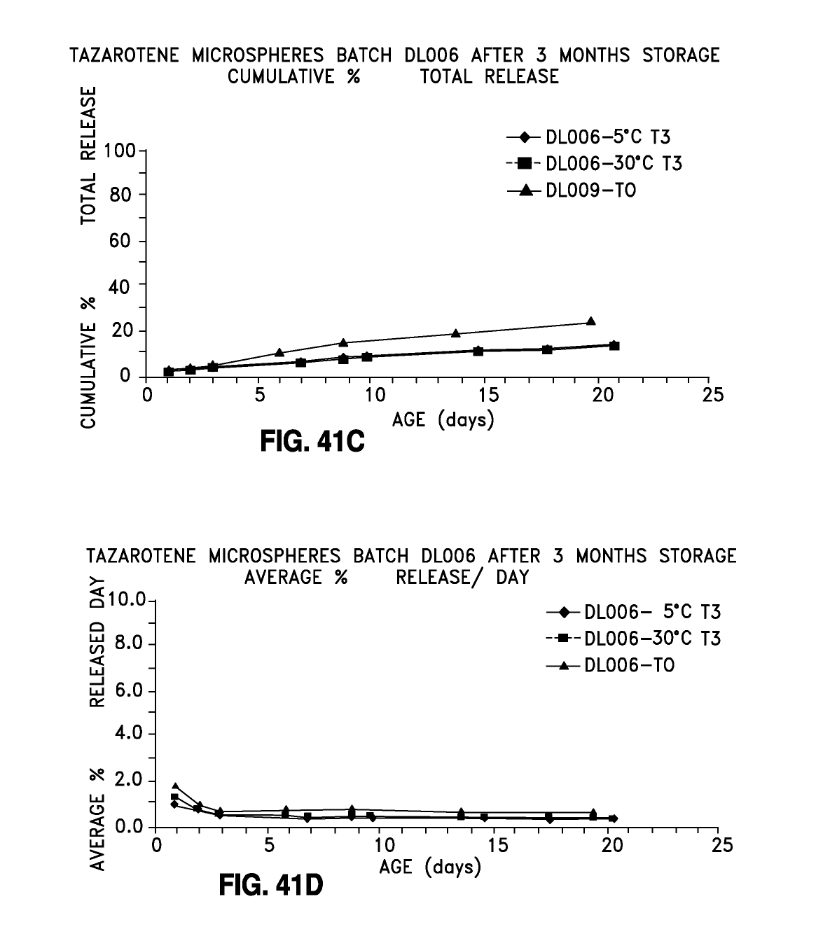

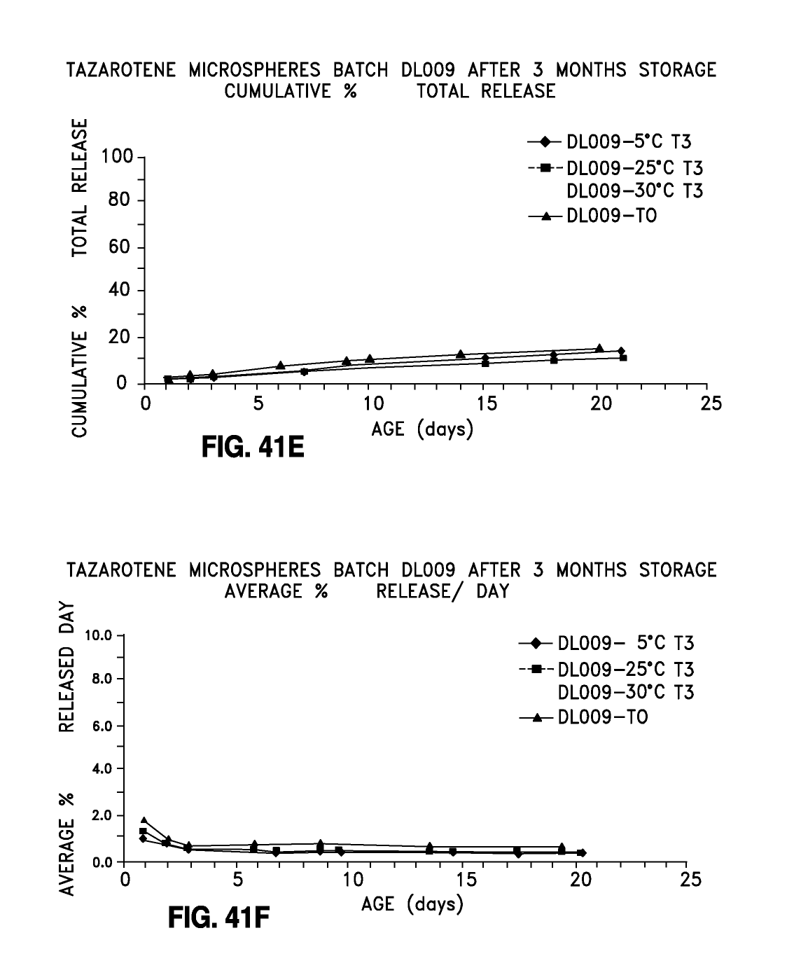

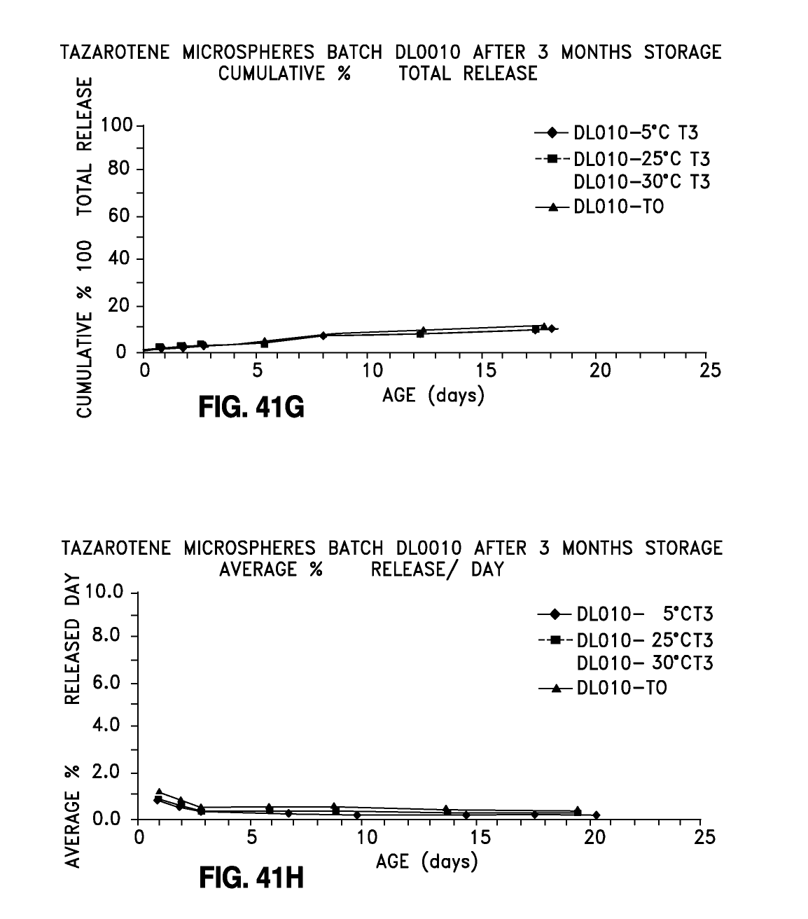

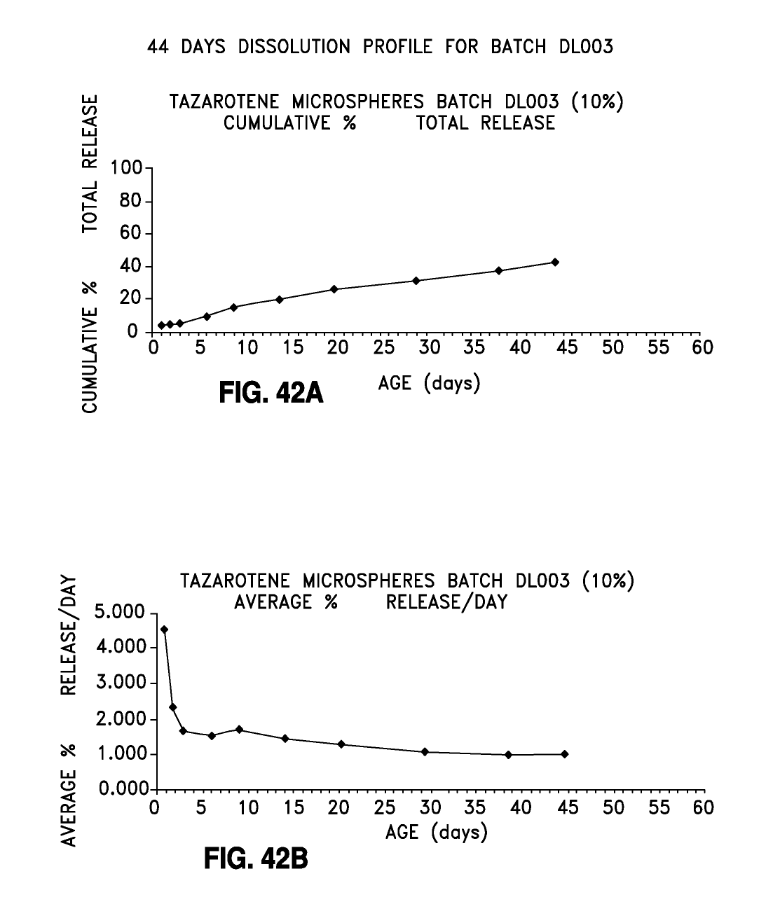

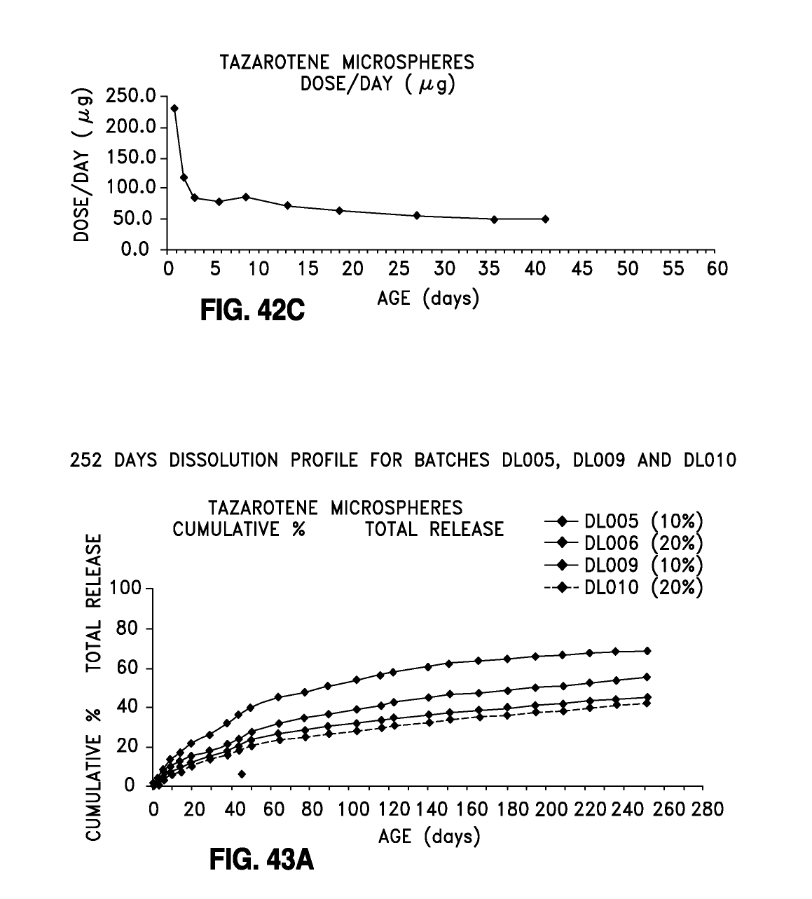

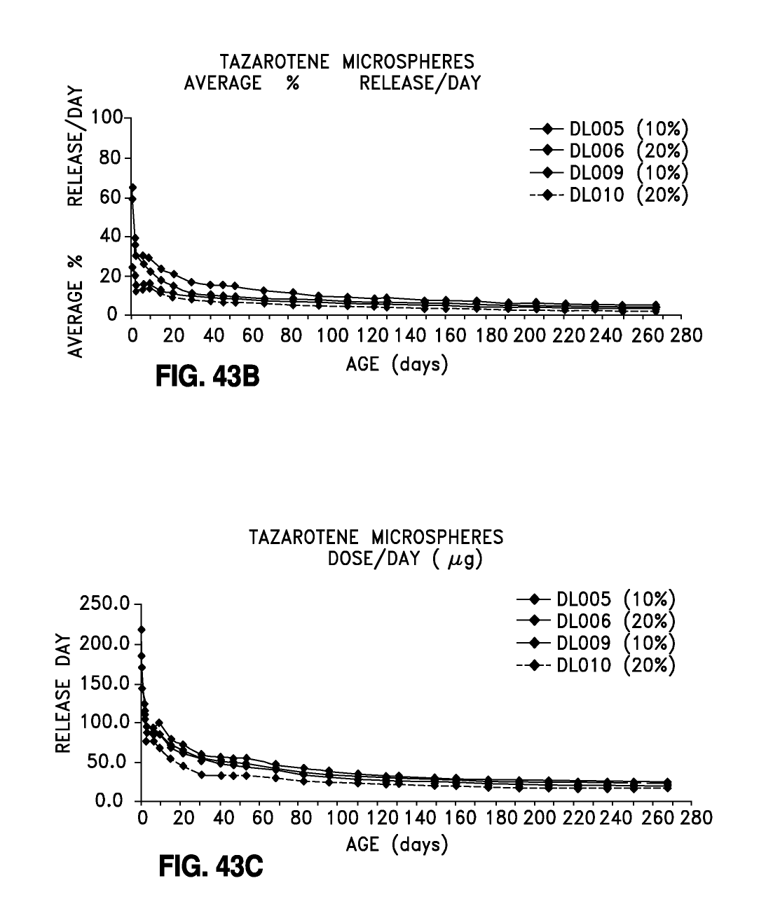

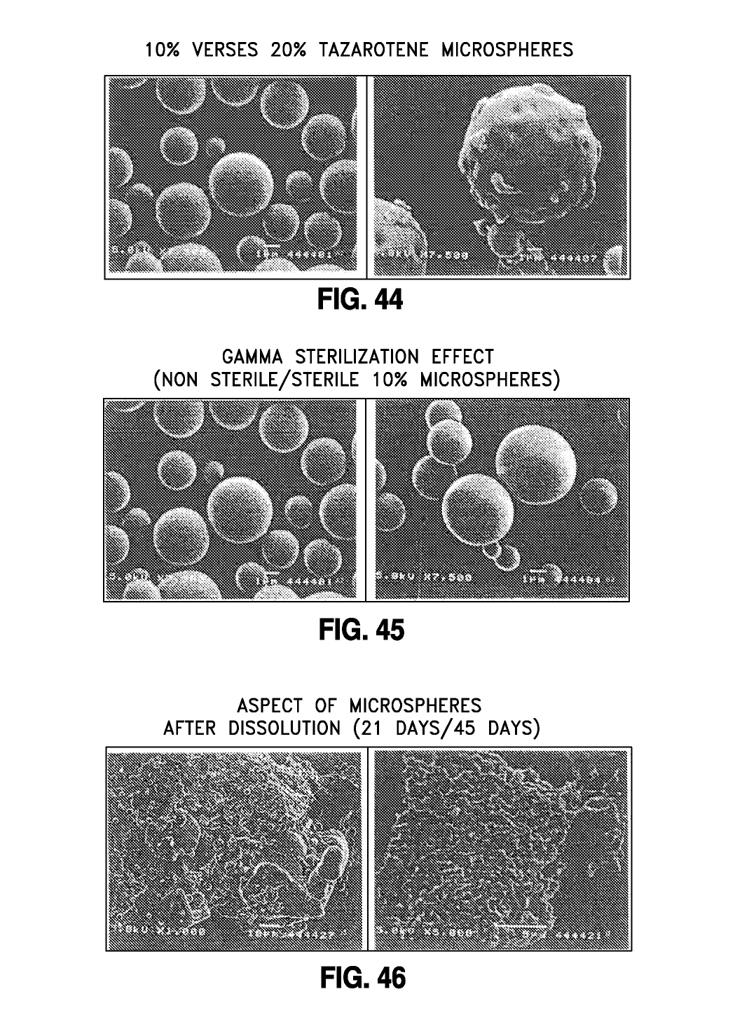

A six-month intraocular pharmacokinetic study of these formulations was initiated in female New Zealand White rabbits. Tissues were assayed following surgical placement of the Tazarotene intravitreal implants. The rabbits received a single bilateral intravitreal implant; a 500 μg Tazarotene dose in a high (formulation #1), medium (formulation #9) or low (formulation #12) release rate formulation. Plasma, vitreous, lens, retina and aqueous humor was assayed on days 4, 8, 14, 21, 31, 57, 113 and 171. Tazarotenic acid, tazarotenic acid is the free acid of the tazarotene ethyl prodrug. Tazarotenic acid is generated rapidly in vivo from tazarotene due to esterases. Hence, tazarotenic acid is monitored in the pharmacokinetic studies. The data show that the implants were successful in sustaining the intravitreal concentrations of tazarotene for six months in vivo. The data from this study is summarized in Table 9 and FIGS. 26 through 29.

TABLE 9
Key pharmacokinetic parameters of TAZAROTENIC ACID in ocular
tissues and plasma following a single implantation of
TAZAROTENE are summarized in the table below:

| | | | | | |
|---|---|---|---|---|---|
| | Aqueous | | | Vitreous | |
| PK Parameters | Humor | Lens | Retina | Humor | Plasma |

Formulation #1 (PLGA/RG502H) - High Release

| | | | | | |
|---|---|---|---|---|---|
| Cₘₐₓ^{a} (ng/mL or ng/g) | 1.08 ± 0.88 | 102 ± 52.7 | 186 ± 60.2 | 110 ± 36.7 | 4.83 |
| Tₘₐₓ (day) | 31 | 57 | 21 | 31 | 21 |
| AUC₀₋ₜₗₐₛₜ^{a,b} (ng · day/mL | 85.7 ± 26.7 | 8370 ± 2230 | 15900 ± 3640 | 8680 ± 1630 | 351 ± 37.3 |
| or ng · day/g) | | | | | |

Formulation #9 (PLGA/RG752) - Medium Release

| | | | | | |
|---|---|---|---|---|---|
| Cₘₐₓ^{a} (ng/mL or ng/g) | 3.37 ± 2.51 | 45.6 ± 10.5 | 115 ± 58.8 | 67.6 ± 40.2 | 3.22 |
| Tₘₐₓ (day) | 57 | 57 | 57 | 57 | 57 |
| AUC₀₋ₜₗₐₛₜ^{a,b} (ng · day/mL | 175 ± 103 | 5490 ± 753 | 14100 ± 2830 | 6480 ± 2410 | 364 ± 28.9 |
| or ng · day/g) | | | | | |

Formulation #12 (PLA/R202H) - Low Release

| | | | | | |
|---|---|---|---|---|---|
| Cₘₐₓ^{a} (ng/mL or ng/g) | 0.173 ± 0.0788 | 61.4 ± 35.8 | 110 ± 36.3 | 75.3 ± 37.3 | 2.61 |
| Tₘₐₓ (day) | 171 | 113 | 171 | 171 | 171 |
| AUC₀₋ₜₗₐₛₜ^{a,b} (ng · day/mL | 18.5 ± 3.69 | 5780 ± 2246 | 9570 ± 2910 | 4540 ± 1180 | 148 ± 58.7 |
| or ng · day/g) | | | | | |

^{a}Mean ± SEM. Per formulation, N = 2 rabbits (4 eyes and 2 plasma) at Cₘₐₓ; N = 16 rabbits (32 eyes) for calculation of AUC₀₋ₜₗₐₛₜ.
^{b}tₗₐₛₜ was Day 57 for all tissues and plasma.

Based on this data a nominal concentration of approximately 1 mg/mL was chosen as a design target. Tazarotene is a more potent RAR agonist and as such effective concentrations are much lower than 1 mg/mL.

### Fastenberg PVR Rating Scale

The five stages of massive periretinal proliferation

| Stage | Characteristics |
|---|---|
| 1 | Intravitreal membrane |
| 2 | Focal traction: |
| | localized vascular changes; hyperemia; engorgement; dilation; |
| | blood vessel elevation |
| 3 | Localized detachment of medullary ray |
| 4 | Extensive retinal detachment; |
| | total medullary ray detachment; peripapillary retinal detachment |
| 5 | Total retinal detachment; retinal folds and holes |

(Fastenberg DM et al, Am J Ophthalmol. 1982; 93: 559-564.)

Retinal Degeneration Models

The objective of this study was to determine the efficacy of various retinoids in preventing retinal damage or improving retinal survival in rhodopsin mutant transgenic rat models of retinal degeneration. The overall goal of the research was to investigate potential photoreceptor survival in mutant rhodopsin transgenic rats using RAR α, RAR β γ, and RXR retinoid agonists.

The experimental design was straightforward. A compound was injected daily to line 3 and every two days for line 4 animals (i.p.) for a given period and animals were sacrificed at the end by CO₂ overdose followed immediately by vascular perfusion of mixed aldehydes. Two litters of rats were used:

1. Transgenic S334ter-3 rats. Line 3 bears a rhodopsin mutation S334ter. Animals of this line exhibit rapid photoreceptor degeneration in the second week after birth. Injection of a compound will be given daily from PD 6 (post-delivery day 6) to PD 20.

2. Transgenic S334ter-4 rats. Line 4 bears a rhodopsin mutation S334ter. Animals of this line experience a 80% loss of photoreceptors by 60 days after birth. The ONL is reduced to one row of nuclei. Injection of a compound will be given every other day from PD 25 to PD 60.

For the line 3 animals, treatment started at PD 6 and the endpoint was PD 20. For the line 4, injection began at PD 25 and ended at PD60 when eyes were harvested. Eyes were embedded in an Epon/Araldite mixture for sectioning at 1 μm thickness along the vertical meridian. Protection of photoreceptors was be evaluated by counting the rows of nuclei in the outer nuclear layer.

The outcome was a measure of retinal outer layer thickness, and cytology over an 8 week period. Compounds included the retinoid agonists tazarotene (RAR β γ), AGN 195183 (RAR α) and AGN 194204 (RXR). Tazarotene was the only compound to show efficacy, indicating an RAR β γ can protect photoreceptors in rhodopsin mutant rats. This is a fairly harsh model and as such the modest improvement by tazarotene is quite significant.

Light Degeneration Model

AGN 194310, AGN 194204, tazarotene and AGN 199852 were evaluated in the blue light retinal degeneration model. Sprague dawley rats were pretreated for five days with daily oral doses of the retinoids. The rats were then exposed to high intensity light, 12000 lux blue fluorescent, for eight hours. Five days post exposure the retinal function was assessed by full flash ERG and structure by outer nuclear layer thickness.

Tazarotene was shown to significantly protect both retinal function and structure. The RXR agonists were not shown to protect retinal function at concentrations below those required to maintain receptor selectivity.

All references, articles, publications and patents and patent applications cited herein are incorporated by reference in their entireties.

While this invention has been described with respect to various specific examples and embodiments, it is to be understood that the invention is not limited thereto and that it can be variously practiced within the scope of the following claims.

We claim:

1. A biodegradable intraocular drug delivery system comprising:
a retinoid component and a biodegradable polymer matrix that releases drug at a rate effective to sustain release of an amount of the retinoid component from the drug delivery system for at least about one week after the drug delivery system is placed in an eye.

2. The system of claim 1, wherein the retinoid component comprises at least one of a retinoid and a retinoid precursor.

3. The system of claim 1, wherein the retinoid component includes an retinoic acid receptor agonist.

4. The system of claim 1, wherein the retinoid component includes a tazarotene, salts thereof, and mixtures thereof.

5. The system of claim 1, wherein the retinoid component includes a tazarotenic acid.

6. The system of claim 1, further comprising an additional ophthalmically acceptable therapeutic agent.

7. The system of claim 1, wherein the retinoid component is dispersed within the biodegradable polymer matrix.

8. The system of claim 1, wherein the matrix comprises at least one polymer selected from the group consisting of polylactides, poly (lactide-co-glycolides), derivatives thereof, and mixtures thereof.

9. The system of claim 1, wherein the system is sterile.

10. The system of claim 1, wherein the matrix comprises a poly (lactide-co-glycolide).

11. The system of claim 1, wherein the matrix comprises a poly(D,L-lactide-co-glycolide).

12. The system of claim 1, wherein the matrix releases drug at a rate effective to sustain release of an amount of the retinoid component from the drug delivery system for more than one month from the time the system is placed in the vitreous of the eye.

13. The system of claim 1, wherein the retinoid component is a tazarotene or tazarotenic acid, and the matrix releases drug at a rate effective to sustain release of a therapeutically effective amount of the tazarotene or tazarotenic acid for a time from about two months to about six months.

14. The system of claim 1, wherein the implant is structured to be placed in the vitreous of the eye.

15. The system of claim 1, wherein the retinoid is tazarotene or tazarotenic acid provided in an amount from about 40% by weight to about 70% by weight of the implant, and the biodegradable polymer matrix comprises a poly (lactide-co-glycolide) in an amount from about 30% by weight to about 60% by weight of the drug delivery system.

16. The system of claim 1 formed as a rod, a wafer, a plug, or a particle.

17. The system of claim 1 which is formed by an extrusion process.

18. A method of making a biodegradable intraocular drug delivery system, comprising the step of: extruding a mixture of a retinoid and a biodegradable polymer component to form a biodegradable material that degrades at a rate effective to sustain release of an amount of the retinoid from the drug delivery system for at least about one week after the drug delivery system is placed in an eye.

19. The method of claim 18, wherein mixture consists essentially of an RAR agonist and a biodegradable polymer.

20. The method of claim 18, further comprising a step of mixing the retinoid with the polymer component before the extrusion step.

21. The method of claim 18, wherein the retinoid component and the polymer component are in a powder form.

22. The method of claim 18, wherein the polymer component comprises a polymer selected from the group consisting of polylactides, poly (lactide-co-glycolides), and combinations thereof.

23. The method of claim 18, wherein the polymer component is substantially free of polyvinyl alcohol.

24. A method of improving or maintaining vision in an eye of a patient, comprising the step of placing a biodegradable intraocular drug delivery system in an eye of the patient, the drug delivery system comprising a retinoid component and a biodegradable polymer matrix, wherein the drug delivery system degrades at a rate effective to sustain release of an amount of the retinoid component from the drug delivery system effective to improve or maintain vision in the eye of the patient.

25. The method of claim 24, wherein the method is effective to treat a retinal ocular condition.

26. The method of claim 24, wherein the ocular condition includes proliferative vitreoretinopathy.

27. The method of claim 24, wherein the drug delivery system is placed in the posterior of the eye.

28. The method of claim 24, wherein the drug delivery system is placed in the eye with a trocar.

29. The method of claim 24, wherein the drug delivery system is placed in the eye with a syringe.

30. The method of claim 24, further comprising a step of administering a therapeutic agent in addition to the retinoid component to the patient.

31. The method of claim 24, wherein the retinoid component includes at least one of tazarotene, tazarotenic acid, salts thereof, and mixtures thereof.
